SEQUENCE LISTING (72 sequences)

SEQ ID NO: 1 - DNA, 111 bp, Artificial Sequence
  Description of Artificial Sequence Synthetic polynucleotide
cactctcagggtaccttcacctctgactactctaaatacctggactctcgtcgtgctcaggacttcgttcagtggctgatgaacaccaaacgtaaccgtaacaacatcgct

SEQ ID NO: 2 - DNA, 183 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggtggcggtcgtggcggtcgtcagctgtactctgctctggctaacaaatgctgccacgttggttgcaccaaacgttctctggctcgtttctgctaa

SEQ ID NO: 3 - DNA, 69 bp, Artificial Sequence
  Description of Artificial Sequence Synthetic oligonucleotide
ctgtactctgctctggctaacaaatgctgccacgttggttgcaccaaacgttctctggctcgtttctgc

SEQ ID NO: 4 - DNA, 87 bp, Artificial Sequence
  Description of Artificial Sequence Synthetic oligonucleotide
gactcttggatggaagaagttatcaaactgtgcggtcgtgaactggttcgtgctcagatcgctatctgcggtatgtctacctggtgc

SEQ ID NO: 5 - DNA, 441 bp, Artificial Sequence
  Description of Artificial Sequence Synthetic polynucleotide
gttccgatccagaaagttcaggacgacaccaaaaccctgatcaaaaccatcgttacccgtatcaacgacatctctcacacccagtctgtttctgctaaacagcgtgttactggtctggacttcatcccgggtctgcacccgatcctgtctctgtctaaaatggaccagaccctggctgtttaccagcaggttctgacctctctgccgtctcagaacgttctgcagatcgctaacgacctggaaaacctgcgtgacctgctgcacctgctggctttctctaaatcttgctctctgccgcagacctctggtctgcagaaaccggaatctctggacggtgttctggaggcttctctgtactctaccgaagttgttgctctgtctcgtctgcagggttctctgcaggacatcctgcagcagctggacgtttctccggaatgctaa

SEQ ID NO: 6 - DNA, 534 bp, Artificial Sequence
  Description of Artificial Sequence Synthetic polynucleotide
gctcctctgggcggtcctgaaccagcacagtacgaggaactgacactgttgttccatggagccttgcagctgggccaggccctcaacggcgtgtaccgcgccacagaggcacgtttgaccgaggccggacacagcctgggtttgtacgacagagccctggagtttctgggtaccgaagtgcgtcagggccaggacgcaactcaggagctgagaacctccctctctgagatccaggtggaggaggacgccctgcacctgcgcgccgaggcgacagcacgctctttgggagaagttgctcgcgctcagcaggccctgcgtgataccgtgcggagactccaagttcagctcagaggcgcttggctcggacaggcgcatcaggagttcgagaccctgaaagctcgtgccgacaaacagtcccacctgctgtgggcgctcaccggtcacgtccagcgccagcaacgcgaaatggccgagcagcagcaatggctgcgccaaatccagcagcgcctgcataccgcggccctgccagcgtaa

SEQ ID NO: 7 - DNA, 549 bp, Artificial Sequence
  Description of Artificial Sequence Synthetic polynucleotide
cacccgatcccggactcttctccgctgctgcagttcggtggtcaggttcgtcagcgttacctgtacaccgacgacgctcagcagaccgaagctcacctggaaatccgtgaagacggtactgttggtggtgctgctgaccagtctccggaatctctgctgcagctgaaagctctgaaaccgggtgttatccagatcctgggtgttaaaacctctcgtttcctgtgccagcgtccggacggtgctctgtacggttctctgcacttcgacccggaggcatgctctttccgtgaacgtctgctggaagacggttacaacgtttaccagtctgaagctcacggtctgccgctgcacctgccgggtaacaaatctccgcaccgtgacccggctccgcgtggtccggctcgtttcctgccgctgccgggtctgccgccggctctgccggaaccgccgggtatcctggctccgcagccgccggacgttggttcttctgacccgctgtctatggttggtggttctcagggtcgttctccgtcttacgaatctccgtaa

SEQ ID NO: 8 - DNA, 330 bp, Artificial Sequence
  Description of Artificial Sequence Synthetic polynucleotide
aacctgggtctggactgcgacgaacactcttctgaatctcgttgctgccgttacccgctgaccgttgacttcgaggcgttcggttgggactggatcatcgctccgaaacgttacaaagctaactactgctctggtcagtgcgaatacatgttcatgcagaaatacccgcacacccacctggttcagcaggctaacccgcgtggttctgctggtccgtgctgcaccccgaccaaaatgtctccgatcaacatgctgtacttcaacgacaaacagcagatcatctacggtaaaatcccgggtatggttgttgaccgttgcggttgctcttaa

SEQ ID NO: 9 - DNA, 1162 bp, Artificial Sequence
  Description of Artificial Sequence Synthetic polynucleotide
ggatccggtggtttcaccatcaaactgctgctgttcatcgttccgctggttatctcttctcgtatcgaccaggacaactcttctttcgactctctgtctccggaaccgaaatctcgtttcgctatgctggacgacgttaaaatcctggctaacggtctgctgcagctgggtcacggtctgaaagacttcgttcacaaaaccaaaggtcagatcaacgacatcttccagaaactgaacatcttcgaccagtctttctacgacctgtctctgcagacctctgaaatcaaagaagaagaaaaagaactgcgtcgtaccacctacaaactgcaggttaaaaacgaagaagttaaaaacatgtctctggaactgaactctaaactggaatctctgctggaagaaaaaatcctgctgcagcagaaagttaaatacctggaagaacagctgaccaacctgatccagaaccagccggaaaccccggaacacccggaagttacctctctgaaaaccttcgttgaaaaacaggacaactctatcaaagacctgctgcagaccgttgaagaccagtacaaacagctgaaccagcagcactctcagatcaaagaaatcgaaaaccagctgcgtcgtacctctatccaggaaccgaccgaaatctctctgtcttctaaaccgcgtgctccgcgtaccaccccgttcctgcagctgaacgaaatccgtaacgttaaacacgacggtatcccggctgaatgcaccaccatctacaaccgtggtgaacacacctctggtatgtacgctatccgtccgtctaactctcaggttttccacgtttactgcgacgttatctctggttctccgtggaccctgatccagcaccgtatcgacggttctcagaacttcaacgaaacctgggaaaactacaaatacggtttcggtcgtctggacggtgaattctggctgggtctggaaaaaatctactctatcgttaaacagtctaactacgttctgcgtatcgaactggaagactggaaagacaacaaacactacatcgaatactctttctacctgggtaaccacgaaaccaactacaccctgcacctggttgctatcaccggtaacgttccgaacgctatcccgaagaagaagaagaaaaaaaagaagaagaaat

SEQ ID NO: 10 - protein, 37 aa, Artificial Sequence
  Description of Artificial Sequence Synthetic polypeptide
HSQGTFTSDYSKYLDSRRAQDFVQWLMNTKRNRNNIA

SEQ ID NO: 11 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 12 - protein, 30 aa, Artificial Sequence
  Description of Artificial Sequence Synthetic polypeptide
HAEGTFTSDVSSYLEGQAAKEFIAWLVKGR

SEQ ID NO: 13 - protein, 29 aa, Artificial Sequence
  Description of Artificial Sequence Synthetic peptide
HSQGTFTSDYSKYLDSRRAQDFVQWLMNT

SEQ ID NO: 14 - protein, 60 aa, Artificial Sequence
  Description of Artificial Sequence Synthetic polypeptide
DSWMEEVIKLCGRELVRAQIAICGMSTWSGGGRGGRQLYSALANKCCHVGCTKRSLARFC

SEQ ID NO: 15 - protein, 23 aa, Artificial Sequence
  Description of Artificial Sequence Synthetic peptide
LYSALANKCCHVGCTKRSLARFC

SEQ ID NO: 16 - protein, 29 aa, Artificial Sequence
  Description of Artificial Sequence Synthetic peptide
DSWMEEVIKLCGRELVRAQIAICGMSTWC

SEQ ID NO: 17 - protein, 146 aa, Artificial Sequence
  Description of Artificial Sequence Synthetic polypeptide
VPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 18 - protein, 177 aa, Artificial Sequence
  Description of Artificial Sequence Synthetic polypeptide
APLGGPEPAQYEELTLLFHGALQLGQALNGVYRATEARLTEAGHSLGLYDRALEFLGTEVRQGQDATQELRTSLSEIQVEEDALHLRAEATARSLGEVARAQQALRDTVRRLQVQLRGAWLGQAHQEFETLKARADKQSHLLWALTGHVQRQQREMAEQQQWLRQIQQRLHTAALPA

SEQ ID NO: 19 - protein, 182 aa, Artificial Sequence
  Description of Artificial Sequence Synthetic polypeptide
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYESP

SEQ ID NO: 20 - protein, 109 aa, Artificial Sequence
  Description of Artificial Sequence Synthetic polypeptide
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 21 - protein, 387 aa, Artificial Sequence
  Description of Artificial Sequence Synthetic polypeptide
GSGGFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPKKKKKKKKKK

SEQ ID NO: 22 - protein, 34 aa, Artificial Sequence
  Description of Artificial Sequence Synthetic polypeptide
INVKCSLPQQCIKPCKDAGMRFGKCMNKKCRCYS

SEQ ID NO: 23 - protein, 36 aa, Artificial Sequence
  Description of Artificial Sequence Synthetic polypeptide
AAAISCVGSPECPPKCRAQGCKNGKCMNRKCKCYYC

SEQ ID NO: 24 - protein, 38 aa, Artificial Sequence
  Description of Artificial Sequence Synthetic polypeptide
  MOD_RES (2)..(2): D-Ser
HSQGTFTSDYSKYLDSRRAQDFVQWLMNTKRNRNNIAC

SEQ ID NO: 25 - protein, 40 aa, Artificial Sequence
  Description of Artificial Sequence Synthetic polypeptide
HGEGTFTSDLSKQMEEEAVRLFIEWLKNGGPSSGAPPPSC

SEQ ID NO: 26 - protein, 52 aa, Artificial Sequence
  Description of Artificial Sequence Synthetic polypeptide
HHHHHHHHENLYFQGSCGGECIGMFKSCDPENDKCCKGRTCSRKHRWCKYKL

SEQ ID NO: 27 - protein, 38 aa, Artificial Sequence
  Description of Artificial Sequence Synthetic polypeptide
GSGGEECIGMFKSCDPENDKCCKGRTCSRKHRWCKYKL

SEQ ID NO: 28 - protein, 38 aa, Artificial Sequence
  Description of Artificial Sequence Synthetic polypeptide
GSCGGECIGMFKSCDPENDKCCKGRTCSRKHRWCKYKL

SEQ ID NO: 29 - protein, 38 aa, Artificial Sequence
  Description of Artificial Sequence Synthetic polypeptide
GSGGECIGMFKSCDPENDKCCKGRTCSRKHRWCKYKLC

SEQ ID NO: 30 - protein, 33 aa, Artificial Sequence
  Description of Artificial Sequence Synthetic polypeptide
ECIGMFKSCDPENDKCCKGRTCSRKHRWCKYKL

SEQ ID NO: 31 - protein, 37 aa, Artificial Sequence
  Description of Artificial Sequence Synthetic polypeptide
ECIGMFKSCDPENDKCCKGRTCSRKHRWCKYKLGGSC

SEQ ID NO: 32 - protein, 78 aa, Artificial Sequence
  Description of Artificial Sequence Synthetic polypeptide
HHHHHHHHENLYFQGSGGDSWMEEVIKLCGRELVRAQIAICGMSTWSGGGRGGRQLYSALANKCCHVGCTKRSLARFC

SEQ ID NO: 33 - protein, 64 aa, Artificial Sequence
  Description of Artificial Sequence Synthetic polypeptide
GSGGDSWMEEVIKLCGRELVRAQIAICGMSTWSGGGRGGRQLYSALANKCCHVGCTKRSLARFC

SEQ ID NO: 34 - protein, 179 aa, Artificial Sequence
  Description of Artificial Sequence Synthetic polypeptide
HHHHHHHHENLYFQGSGGDSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 35 - protein, 46 aa, Artificial Sequence
  Description of Artificial Sequence Synthetic polypeptide
HHHHHHHHENLYFQSGGDSWMEEVIKLCGRELVRAQIAICGMSTWS

SEQ ID NO: 36 - protein, 24 aa, Artificial Sequence
  Description of Artificial Sequence Synthetic peptide
QLYSALANKCCHVGCTKRSLARFC

SEQ ID NO: 37 - protein, 78 aa, Artificial Sequence
  Description of Artificial Sequence Synthetic polypeptide
HHHHHHHHENLYFQGSGGDSWMEEVIKLCGRELVRAQIAICGMSTWCGGGRGGRQLYSALANKCCHVGCTKRSLARFC

SEQ ID NO: 38 - protein, 179 aa, Artificial Sequence
  Description of Artificial Sequence Synthetic polypeptide
HHHHHHHHENLYFQGSGGDSWMEEVIKLCGRELVRAQIAICGMSTWCKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 39 - protein, 72 aa, Artificial Sequence
  Description of Artificial Sequence Synthetic polypeptide
MIEEGRDSWMEEVIKLCGRELVRAQIAICGMSTWSKRKPTGYGSRKKRQLYSALANKCCHVGCTKRSLARFC

SEQ ID NO: 40 - protein, 29 aa, Artificial Sequence
  Description of Artificial Sequence Synthetic peptide
DSWMEEVIKLCGRELVRAQIAICGMSTWS

SEQ ID NO: 41 - protein, 23 aa, Artificial Sequence
  Description of Artificial Sequence Synthetic peptide
LYSALANKCCHVGCTKRSLARFC

SEQ ID NO: 42 - protein, 24 aa, Artificial Sequence
  Description of Artificial Sequence Synthetic peptide
QLYSALANKCCHVGCTKRSLARFC

SEQ ID NO: 43 - protein, 24 aa, Artificial Sequence
  Description of Artificial Sequence Synthetic peptide
QLYSCLANKCCHVGCTKRSLARFC

SEQ ID NO: 44 - protein, 24 aa, Artificial Sequence
  Description of Artificial Sequence Synthetic peptide
QLYSALANKCCKVGCTKRSLARFC

SEQ ID NO: 45 - protein, 24 aa, Artificial Sequence
  Description of Artificial Sequence Synthetic peptide
CLYSALANKCCHVGCTKRSLARFC

SEQ ID NO: 46 - protein, 24 aa, Artificial Sequence
  Description of Artificial Sequence Synthetic peptide
ALYSALANKCCAVGCTKRSLARFC

SEQ ID NO: 47 - protein, 24 aa, Artificial Sequence
  Description of Artificial Sequence Synthetic peptide
ALYSALANKCCAVGCTKRSLARFC

SEQ ID NO: 48 - protein, 29 aa, Artificial Sequence
  Description of Artificial Sequence Synthetic peptide
ASWMEEVIKLCGRELVRAQIAICGMSTWS

SEQ ID NO: 49 - protein, 29 aa, Artificial Sequence
  Description of Artificial Sequence Synthetic peptide
ASWMEEVIKLCGRELVRAQIAICGMSTWC

SEQ ID NO: 50 - protein, 29 aa, Artificial Sequence
  Description of Artificial Sequence Synthetic peptide
ASWKEEVIKLCGRELVRAQIAICGKSTWC

SEQ ID NO: 51 - protein, 29 aa, Artificial Sequence
  Description of Artificial Sequence Synthetic peptide
ASWKEEVIKLCGRELVRAQIAICGMSTWC

SEQ ID NO: 52 - protein, 29 aa, Artificial Sequence
  Description of Artificial Sequence Synthetic peptide
CSWMEEVIKLCGRELVRAQIAICGMSTWS

SEQ ID NO: 53 - protein, 24 aa, Artificial Sequence
  Description of Artificial Sequence Synthetic peptide
  MOD_RES (1)..(1): Gln, Glu or pyro-Glu
XLYSALANKCCHVGCTKRSLARFC

SEQ ID NO: 54 - protein, 24 aa, Artificial Sequence
  Description of Artificial Sequence Synthetic peptide
QCYSALANKCCHVGCTKRSLARFC

SEQ ID NO: 55 - protein, 24 aa, Artificial Sequence
  Description of Artificial Sequence Synthetic peptide
QLYSALANKCCHCGCTKRSLARFC

SEQ ID NO: 56 - protein, 29 aa, Artificial Sequence
  Description of Artificial Sequence Synthetic peptide
DSWMEEVIKCLGRELVRAQIAICGMSTWS

SEQ ID NO: 57 - protein, 24 aa, Artificial Sequence
  Description of Artificial Sequence Synthetic peptide
QLYSALANKCCHVGCTKCSLARFC

SEQ ID NO: 58 - protein, 29 aa, Artificial Sequence
  Description of Artificial Sequence Synthetic peptide
ASWMEEVIKLCGRELVRAQIAICGKSTWC

SEQ ID NO: 59 - protein, 24 aa, Artificial Sequence
  Description of Artificial Sequence Synthetic peptide
  MOD_RES (1)..(1): pyro-Glu
XCYSALANKCCHVGCTKRSLARFC

SEQ ID NO: 60 - protein, 24 aa, Artificial Sequence
  Description of Artificial Sequence Synthetic peptide
  MOD_RES (1)..(1): Gln, Glu, pyro-Glu or Ala
  MOD_RES (2)..(2): Cys, Leu, Ala, Ile or Val
  MOD_RES (5)..(5): Cys, Leu, Ala, Ile or Val
  MOD_RES (12)..(12): His, Ala or Lys
  MOD_RES (13)..(13): Cys, Leu, Ala, Ile or Val
  MOD_RES (18)..(18): Arg or Cys
  See specification as filed for detailed description of substitutions and preferred embodiments
XXYSXLANKCCXXGCTKXSLARFC

SEQ ID NO: 61 - protein, 29 aa, Artificial Sequence
  Description of Artificial Sequence Synthetic peptide
ASWMEECIKLCGRELVRAQIAICGMSTWS

SEQ ID NO: 62 - DNA, 60 bp, Artificial Sequence
  Description of Artificial Sequence Synthetic primer
tatttccagggatccggtggtgactcttggatggaagaagttatcaaactgtgcggtcgt

SEQ ID NO: 63 - DNA, 60 bp, Artificial Sequence
  Description of Artificial Sequence Synthetic primer
tagacataccgcagatagcgatctgagcacgaaccagttcacgaccgcacagtttgataa

SEQ ID NO: 64 - DNA, 60 bp, Artificial Sequence
  Description of Artificial Sequence Synthetic primer
cgctatctgcggtatgtctacctggtctggtggcggtcgtggcggtcgtcagctgtactc

SEQ ID NO: 65 - DNA, 60 bp, Artificial Sequence; identical to SEQ ID NO: 64
  Description of Artificial Sequence Synthetic primer

SEQ ID NO: 66 - DNA, 60 bp, Artificial Sequence; identical to SEQ ID NO: 63
  Description of Artificial Sequence Synthetic primer

SEQ ID NO: 67 - DNA, 60 bp, Artificial Sequence; identical to SEQ ID NO: 64
  Description of Artificial Sequence Synthetic primer

SEQ ID NO: 68 - DNA, 60 bp, Artificial Sequence
  Description of Artificial Sequence Synthetic primer
tttggtgcaaccaacgtggcagcatttgttagccagagcagagtacagctgacgaccgcc

SEQ ID NO: 69 - DNA, 29 bp, Artificial Sequence
  Description of Artificial Sequence Synthetic primer
aatctgtatttccagggatccggtggtga

SEQ ID NO: 70 - DNA, 54 bp, Artificial Sequence
  Description of Artificial Sequence Synthetic primer
tggctaagctttagcagaaacgagccagagaacgtttggtgcaaccaacgtggc

SEQ ID NO: 71 - protein, 6 aa, Artificial Sequence
  Description of Artificial Sequence Synthetic peptide
ENLYFQ

SEQ ID NO: 72 - protein, 6 aa, Artificial Sequence
  Description of Artificial Sequence Synthetic peptide
GGGRGG

(19) United States
(12) Patent Application Publication
(10) Pub. No.: US 2018/0207276 A1
(43) Pub. Date: Jul. 26, 2018

(54) MODIFIED THERAPEUTIC AGENTS AND COMPOSITIONS THEREOF

(71) Applicant: THE CALIFORNIA INSTITUTE FOR BIOMEDICAL RESEARCH, La Jolla, CA (US)

(72) Inventors: Weijun SHEN, San Diego, CA (US); Avinash MUPPIDI, Indianapolis, IN (US); Peter G. SCHULTZ, La Jolla, CA (US)

(21) Appl. No.: 15/557,986

(22) PCT Filed: Mar. 17, 2016
(86) PCT No.: PCT/US16/22880

§ 371 Date: Sep. 13, 2017

Related U.S. Application Data
(60) Provisional application No. 62/134,939, filed on Mar. 18, 2015.

(51) Int. Cl. A61K 47/12; A61K 38/22; A61P 29/00; A61K 47/14

(52) U.S. Cl. CPC A61K 47/12; A61K 47/14; A61P 29/00; A61K 38/2221

(57) ABSTRACT

Methods and compositions are provided for extending the half-life of a therapeutic agent. One half-life extending moieties may be attached to a therapeutic agent, thereby extending the half life of the therapeutic agent. The modified therapeutic agents comprising a half-life extending moieties attached to a therapeutic agent may be used to treat a disease or condition in a subject in need thereof.

### CROSS-REFERENCE TO RELATED APPLICATIONS

This application is a U.S. National Stage entry of International Application No. PCT/US16/22880, filed Mar. 17, 2016, which claims the benefits of U.S. Application Ser. No. 62/134,939, filed Mar. 18, 2015, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Apr. 25, 2016, is named 41135-739.831_SL.txt and is 41.6 KB in size.

### BACKGROUND OF THE INVENTION

The development of therapeutic agents (e.g., biological drugs) is often hampered by short half-lives. The biological half-life or elimination half-life of a substance is the time it takes for a substance (for example a metabolite, drug, signaling molecule, radioactive nuclide, or other substance) to lose half of its pharmacologic, physiologic, or radiologic activity. As a result of the short half-life, patients are often administered higher dosages more frequently, which may lead to reduced compliance, higher costs and greater risks of side effects.

Extended-release products are designed to prolong the absorption of drugs with short half-lives, thereby allowing longer dosing intervals while minimizing fluctuations in serum drug levels. Current strategies used for extending half-lives are those that increase hydrodynamic volume (PEGylation) or those that use FcRn-mediated recycling (albumin fusions). Attachment of polypeptides or lipophilic constituents to drugs has also been used to extend the half-life of a biological agent (U.S. Pat. No. 6,268,343; U.S. Pat. No. 5,750,497; U.S. Pat. No. 8,129,343).

The present disclosure provides modified therapeutic agents for improving the biological, chemical, physiologic, pharmacologic, pharmacokinetic, and/or pharmacodynamic properties of a therapeutic agent.

### SUMMARY OF THE INVENTION

Disclosed herein are modified therapeutic agents comprising:
(i) a therapeutic agent (TA) comprising a relaxin A-chain amino acid sequence selected from the group consisting of SEQ ID NO: 54, 55, 59 and 60; and
(ii) a half-life extending moiety;
   wherein the TA is covalently attached to the half-life extending moiety via a cysteine residue of the TA.

In some embodiments of a modified therapeutic agent, the cysteine residue is in the relaxin A-chain amino acid sequence. In some embodiments of a modified therapeutic agent, the cysteine residue is at the 2 position of the relaxin A-chain amino acid sequence. In some embodiments of a modified therapeutic agent, the therapeutic agent comprises the relaxin A-chain amino acid sequence SEQ ID NO: 54. In some embodiments of a modified therapeutic agent, the therapeutic agent comprises the relaxin A-chain amino acid sequence SEQ ID NO: 59. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, or substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a relaxin A-chain amino acid sequence that is at least 50% homologous to an amino acid sequence selected from the group consisting of SEQ ID NO: 54, 55, 59 and 60; and
(ii) a half-life extending moiety;
   wherein the TA is covalently attached to the half-life extending moiety via a cysteine residue of the TA.

In some embodiments of a modified therapeutic agent, the relaxin A-chain amino acid sequence is at least 80% homologous to an amino acid sequence selected from the group consisting of SEQ ID NO: 54, 55, 59 and 60. In some embodiments of a modified therapeutic agent, the relaxin A-chain amino acid sequence is at least 90% homologous to an amino acid sequence selected from the group consisting of SEQ ID NO: 54, 55, 59 and 60. In some embodiments of a modified therapeutic agent, the cysteine residue is in the relaxin A-chain amino acid sequence. In some embodiments of a modified therapeutic agent, the cysteine residue is at the 2 position of the relaxin A-chain amino acid sequence. In some embodiments of a modified therapeutic agent, the relaxin A-chain amino acid sequence is at least 50% homologous to the amino acid sequence SEQ ID NO: 54. In some embodiments of a modified therapeutic agent, the relaxin A-chain amino acid sequence is at least 50% homologous to the amino acid sequence SEQ ID NO: 59. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, or substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a relaxin A-chain amino acid sequence selected from the group consisting of SEQ ID NO: 54, 55, 59 and 60; wherein up to five amino acids have been added, deleted, substituted, or a combination thereof; and
(ii) a half-life extending moiety;
   wherein the TA is covalently attached to the half-life extending moiety via a cysteine residue of the TA.

In some embodiments of a modified therapeutic agent, one amino acid has been added, deleted, or substituted from the relaxin A-chain amino acid sequence. In some embodiments of a modified therapeutic agent, two amino acids have been added, deleted, substituted, or a combination thereof; from the relaxin A-chain amino acid sequence. In some embodiments of a modified therapeutic agent, three amino acids have been added, deleted, substituted, or a combination thereof; from the relaxin A-chain amino acid sequence. In some embodiments of a modified therapeutic agent, the cysteine residue is in the relaxin A-chain amino acid sequence. In some embodiments of a modified therapeutic agent, the cysteine residue is at the 2 position of the relaxin A-chain amino acid sequence. In some embodiments of a modified therapeutic agent, the relaxin A-chain amino acid sequence is SEQ ID NO: 54 wherein up to five amino acids have been added, deleted, substituted, or a combination thereof. In some embodiments of a modified therapeutic agent, the relaxin A-chain amino acid sequence is SEQ ID NO: 59 wherein up to five amino acids have been added, deleted, substituted, or a combination thereof. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, or substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises the relaxin B-chain amino acid sequence SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a portion of a relaxin A-chain amino acid sequence selected from the group consisting of SEQ ID NO: 54, 55, 59 and 60; and
(ii) a half-life extending moiety;
   wherein the TA is covalently attached to the half-life extending moiety via a cysteine residue of the TA.

In some embodiments of a modified therapeutic agent, the portion of the relaxin A-chain amino acid sequence comprises at least 20 consecutive amino acids of SEQ ID NO: 54, 55, 59 or 60. In some embodiments of a modified therapeutic agent, the portion of the relaxin A-chain amino acid sequence comprises at least 15 consecutive amino acids of SEQ ID NO: 54, 55, 59 and 60. In some embodiments of a modified therapeutic agent, the portion of the relaxin A-chain comprises at least 10 consecutive amino acids of SEQ ID NO: 54, 55, 59 and 60. In some embodiments of a modified therapeutic agent, the cysteine residue is in the portion of the relaxin A-chain amino acid sequence. In some embodiments of a modified therapeutic agent, the cysteine residue is at the 2 position of the portion of the relaxin A-chain amino acid sequence. In some embodiments of a modified therapeutic agent, the therapeutic agent comprises a portion of the relaxin A-chain amino acid sequence SEQ ID NO: 54. In some embodiments of a modified therapeutic agent, the therapeutic agent comprises a portion of the relaxin A-chain amino acid sequence SEQ ID NO: 59. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, or substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises the amino acid sequence SEQ ID NO: 48. In some embodiments of a modified therapeutic agent, the half-life of the modified therapeutic agent is longer than the half-life of the therapeutic agent alone.

In some embodiments of a modified therapeutic agent, the modified therapeutic agent has the following formula:

TA-A¹-P¹-L Formula (A)

wherein:
TA is the therapeutic agent;

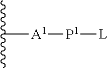

is the half-life extending moiety.

In some embodiments of a modified therapeutic agent,
A¹ is a chemical group linking TA and P¹ or L;
P¹ is a bond or -PEG-A²-; and
L is a lipid derivative selected from the group consisting of sterol derivatives, bile acid derivatives, vitamin E derivatives, fatty di-acid derivatives, fatty acid derivatives, fatty amide derivatives, fatty amine amine derivatives, and fatty alcohol derivatives; wherein the lipid derivative is linked to P¹ via an amide or an ether linkage.

In some embodiments of a modified therapeutic agent, a sulfur atom of the cysteine residue of the TA is connected to A¹ via a chemical bond.

In some embodiments of a modified therapeutic agent of Formula (A),
A′ is selected from

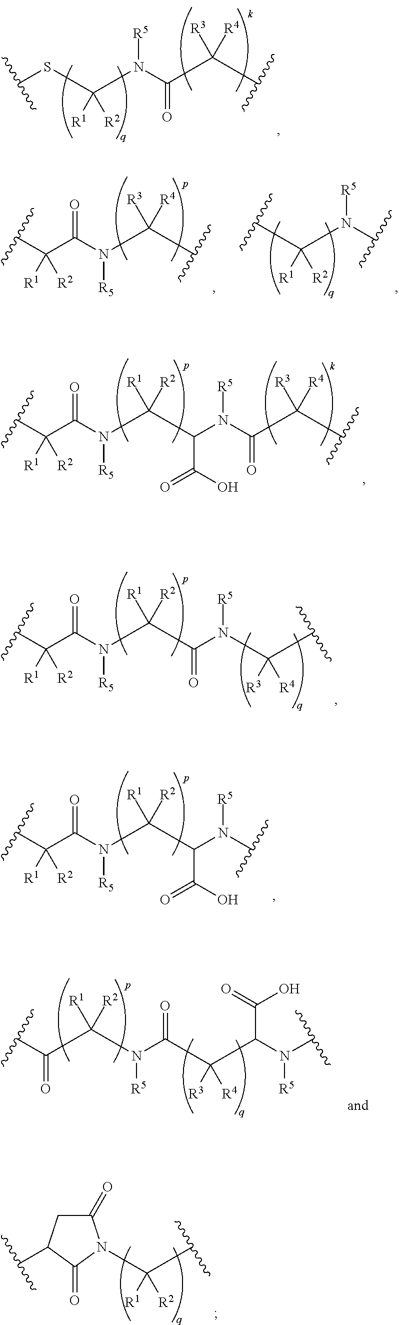

PEG is selected from

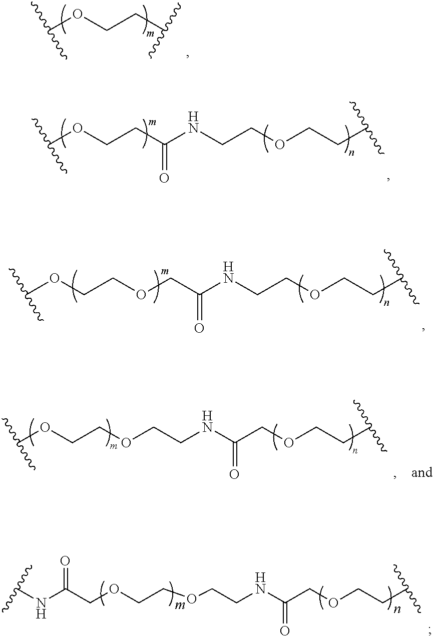

A² is selected from a bond,

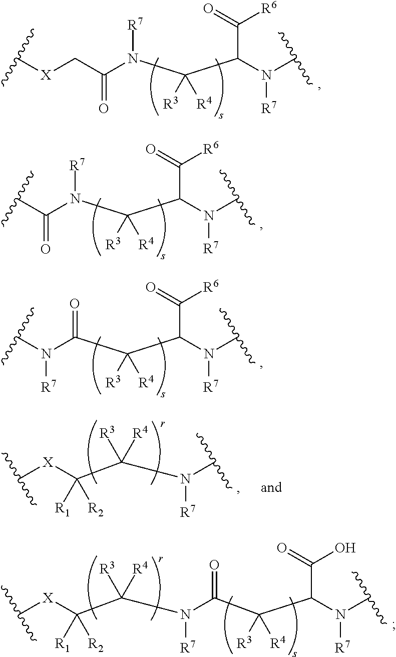

L is an acid moiety selected from tetradecanoic acid, octadecanedioic acid, myristic acid, stearic acid, docosahexaenoic acid, lithocholic acid, lithocholic acid, cholic acid, and palmitic acid; wherein the acid moiety is linked to P¹ via an amide linkage;
X is a bond, —N(R⁵)—, —S—, or —O—;
each R¹, R², R³, and R⁴ is independently H, halo, —CN, —SR⁵, alkyl, cycloalkyl, haloalkyl, —NR⁵R⁵, or —OR⁵;
each R⁵ is independently H, alkyl, haloalkyl, arylalkyl, or heteroalkyl;
R⁶ is OH or —NR⁵R⁵;
each R⁷ is independently H, alkyl, haloalkyl, arylalkyl, or heteroalkyl;
m and n are independently 1, 2, 3, 4, 5, 6, 7, 8, 9, 10, 11, 12, 13, 14, 15, 16, 17, 18, 19, or 20;
r is 1, 2, 3, 4, 5, 6, 7, 8, 9, or 10;
s is 1, 2, 3, 4, or 5;
k is 1, 2, 3, 4, 5, 6, 7, 8, 9, or 10;
p is 2, 3, 4, 5, 6, 7, 8, 9, or 10; and
q is 2, 3, 4, 5, 6, 7, 8, 9, or 10.

In some embodiments of a modified therapeutic agent, A¹ is

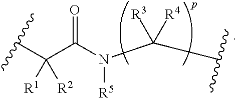

In some embodiments of a modified therapeutic agent, PEG is

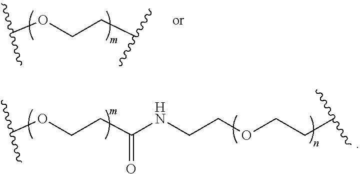

In some embodiments of a modified therapeutic agent, A² is

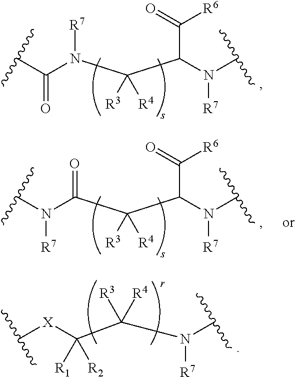

In some embodiments of a modified therapeutic agent, each R¹, R², R³, and R⁴ is H. In some embodiments of a modified therapeutic agent, each R⁵ is independently H. In some embodiments of a modified therapeutic agent, each R⁶ is OH or NH₂. In some embodiments of a modified therapeutic agent, each R⁷ is H. In some embodiments of a modified therapeutic agent, m is 1, 2, 3, 4, 5, 6, 7, 8, or 9. In some embodiments of a modified therapeutic agent, n is 1, 2, 3, 4, or 5. In some embodiments of a modified therapeutic agent, r is 1, 2, or 3. In some embodiments of a modified therapeutic agent, s is 2 or 4. In some embodiments of a modified therapeutic agent, p is 2. In some embodiments of a modified therapeutic agent, X is —O—.

In another aspect are pharmaceutical compositions comprising one or more of the modified therapeutic agents described herein.

Further disclosed herein are methods for treating a disease or condition in a subject in need thereof, the method comprising administering to the subject a composition comprising a therapeutically effective amount of the modified therapeutic agent described herein. The disease or condition may be a cardiovascular disorder. The disease or condition may be acute heart failure. The disease or condition may be fibrosis. The disease or condition may be pain. The pain may be neuropathic pain or inflammatory pain. The method may further comprise administering to the subject one or more additional therapeutic agents. The one or more additional therapeutic agents may be selected from the group consisting of an anti-inflammatory drug, a statin, a diuretic, a beta-blocker, an angiotensin converting enzyme inhibitor, an angiotensin II receptor blocker or any combination thereof. The one or more additional therapeutic agents may be selected from the group consisting of an anti-inflammatory drug, a drug to treat pain, or any combination thereof. The one or more additional therapeutic agents may be aspirin.

### BRIEF DESCRIPTION OF THE DRAWINGS

The novel features of the invention are set forth with particularity in the appended claims. The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows relaxin modified with cysteine for lipid conjugation, (boxed residues may be mutated, for example to L-cysteine, lysine, glutamine, alanine).

FIG. 2 shows human relaxin 2 activating relaxin 2 receptor (RXFP2 or LGR7) in HEK293T cells expressing LGR7 and CRE-Luc reporter line.

FIG. 3 shows a chromatogram of the showing the conversion of Relaxin-B:D1A-A:Q1-L2C-FA2 agent (entry 1, Table 3) to Relaxin-B:D1A-A:pQ1-L2C-FA2 (entry 5, Table 3).

FIG. 4 shows a schematic of a pVB008 relaxin2 linker vector and additional schematics of other relaxin expression constructs. FIG. 4 discloses “GGGRGG” as SEQ ID NO: 72.

FIG. 5 shows (A) the disulfide bond pattern for the Toxin-550 peptide (wild-type), and (B) an exemplary in vitro folding pathway of cysteine-knot Toxin-550 peptide, the middle structure depicting a two-sulfide bond intermediate, in which a disulfide bond is formed between cysteine I and cysteine IV and between cysteine II and cysteine V.

FIG. 6 shows mouse pharmacokinetic data for wild-type relaxin.

FIG. 7 shows mouse pharmacokinetic data for a modified therapeutic agent (entry 2, Table 3).

FIG. 8 shows mouse pharmacokinetic data for a modified therapeutic agent (entry 1, Table 3).

FIG. 9 shows mouse pharmacokinetic data for a modified therapeutic agent (entry 3, Table 3).

FIG. 10 shows mouse pharmacokinetic data for a modified therapeutic agent (A: IV, B: SC) (entry 7, Table 3).

FIG. 11 shows mouse pharmacokinetic data for a modified therapeutic agent (A: IV, B: SC) (entry 8, Table 3).

FIG. 12 shows mouse pharmacokinetic data for a modified therapeutic agent (A: IV, B: SC) (entry 9, Table 3).

FIG. 13 shows rat pharmacokinetic data for a modified therapeutic agent (A: IV, B: SC) (entry 5, Table 3).

FIG. 14 shows a modified therapeutic agent activating relaxin 2 receptor (RXFP2 or LGR7) in HEK293T cells expressing LGR7 and CRE-Luc reporter line compared to wild type relaxin (entry 9, Table 3).

FIG. 15 shows a modified therapeutic agent activating relaxin 2 receptor (RXFP2 or LGR7) in HEK293T cells expressing LGR7 and CRE-Luc reporter line compared to wild type relaxin (entry 1, Table 3).

FIG. 16 shows a modified therapeutic agent activating relaxin 2 receptor (RXFP2 or LGR7) in HEK293T cells expressing LGR7 and CRE-Luc reporter line compared to wild type relaxin (entry 7, Table 3).

FIG. 17 shows a modified therapeutic agent activating relaxin 2 receptor (RXFP2 or LGR7) in HEK293T cells expressing LGR7 and CRE-Luc reporter line compared to wild type relaxin (entry 8, Table 3).

FIG. 18 shows a modified therapeutic agent activating relaxin 2 receptor (RXFP2 or LGR7) in HEK293T cells expressing LGR7 and CRE-Luc reporter line compared to wild type relaxin (entry 4, Table 3).

### DETAILED DESCRIPTION OF THE INVENTION

Disclosed herein are modified therapeutic agents. Generally, the modified therapeutic agent may comprise a therapeutic agent (TA) and one or more half-life extending moieties, wherein the TA is attached to the one or more half-life extending moieties. In some embodiments, the modified therapeutic agent comprises a therapeutic agent (TA) and one half-life extending moieties, wherein the TA is attached to the half-life extending moieties. The therapeutic agent may be a peptide. The TA may be covalently attached to each of the one or more half-life extending moieties. The TA may be attached to the one or more half-life extending moieties via an amino acid residue on the TA. The amino acid residue may be a cysteine or a lysine. The amino acid residue may be a cysteine. The half-life of the modified therapeutic agent may be longer than the half-life of the therapeutic agent alone. A half-life extending moiety may comprise a lipid, a polyglycol region, or a combination thereof. The half-life extending moiety may further comprise a heteroaryl or heterocyclyl group. A half-life extending moiety may be attached to the sulfur atom of a cysteine residue on the TA. The TA may be a peptide comprising at least one isosteric disulfide mimetic.

Disclosed herein are lipid conjugates (LCs). Generally, the LCs comprise a lipid and a therapeutic agent. The LCs may further comprise a linker connecting the lipid to the therapeutic agent, the linker comprising a heteroaryl or heterocyclyl group. The therapeutic agent may comprise at least one isosteric disulfide mimetic.

The lipid conjugates (LCs) may comprise (a) one or more lipids, the lipids selected from sterols, sterol derivatives, bile acids, vitamin E derivatives, fatty di-acids, fatty acids, fatty amides, fatty amines, and fatty alcohols, and derivatives thereof; (b) a therapeutic agent (TA); and (c) a linker connecting each of the one or more lipids to the TA, wherein the linker comprises a heteroaryl or heterocyclyl group; the TA is a peptide; and each linker is conjugated to the TA via an amino acid on the peptide. The amino acid residue may be cysteine or lysine.

The lipid conjugates (LCs) may comprise (a) one or more lipids, the lipids selected from sterols, sterol derivatives, bile acids, vitamin E derivatives, fatty di-acids, fatty acids, fatty amides, and fatty alcohols; (b) one or more therapeutic agents (TAs); and (c) a linker connecting the lipid to the TA, wherein the linker comprises a heteroaryl or heterocyclyl group and each linker is conjugated to the TA via an amino acid residue. The amino acid residue may be cysteine or lysine.

Further disclosed herein is a lipid conjugate (LC) comprising (a) one or more lipids, the lipids selected from sterols, sterol derivatives, bile acids, vitamin E derivatives, fatty di-acids, fatty acids, fatty amides, and fatty alcohols; (b) one or more therapeutic agents (TAs) and (c) a linker connecting the lipid to the TA, wherein the linker comprises a heteroaryl or heterocyclyl group, the one or more TAs comprise a relaxin peptide or derivative thereof, and each linker is attached to the one or more TAs via an amino acid residue.

Further disclosed herein is a lipid conjugate (LC) comprising (a) one or more lipids, the lipids selected from sterols, sterol derivatives, bile acids, vitamin E derivatives, fatty di-acids, fatty acids, fatty amides, and fatty alcohols; and (b) one or more therapeutic agents (TAs) comprising at least one isosteric disulfide mimetic, wherein each of the one or more TAs is a peptide and the one or more lipids are conjugated to the TA via an amino acid residue on the peptide.

Before the present methods, kits and compositions are described in greater detail, it is to be understood that this invention is not limited to particular method, kit or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided, it is understood that each intervening value, to the terth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods and compositions are provided for producing modified therapeutic agents that extend the half-life of a therapeutic agent. These methods and compositions find therapeutic use in a number of diseases, for example, cardiovascular disease may be more effectively treated with a half-life extension molecule conjugated to relaxin than by relaxin alone. These and other objects, advantages, and features of the invention will become apparent to those persons skilled in the art upon reading the details of the compositions and methods as more fully described below.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Modified Therapeutic Agent

Disclosed herein are modified therapeutic agents comprising:
(i) a therapeutic agent (TA) comprising a relaxin A-chain; and
(ii) a half-life extending moiety;
   wherein the TA is covalently attached to the half-life extending moiety via a cysteine residue of the TA.

In some embodiments of a modified therapeutic agent, the relaxin A-chain is an amino acid sequence selected from the group consisting of SEQ ID NO: 54, 55, 59 and 60. In some embodiments of a modified therapeutic agent, the relaxin A-chain amino acid sequence is not SEQ ID NO: 41. In some embodiments of a modified therapeutic agent, the relaxin A-chain amino acid sequence is not SEQ ID NO: 42. In some embodiments of a modified therapeutic agent, the relaxin A-chain amino acid sequence is not SEQ ID NO: 43. In some embodiments of a modified therapeutic agent, the relaxin A-chain amino acid sequence is not SEQ ID NO: 44. In some embodiments of a modified therapeutic agent, the relaxin A-chain amino acid sequence is not SEQ ID NO: 45. In some embodiments of a modified therapeutic agent, the relaxin A-chain amino acid sequence is not SEQ ID NO: 46. In some embodiments of a modified therapeutic agent, the relaxin A-chain amino acid sequence is not SEQ ID NO: 47. In some embodiments of a modified therapeutic agent, the relaxin A-chain amino acid sequence is not SEQ ID NO: 53. In some embodiments of a modified therapeutic agent, the relaxin A-chain amino acid sequence is not SEQ ID NO: 55. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, substituted, or a combination thereof. In some embodiments of a modified therapeutic agent, the therapeutic agent further comprises a modified relaxin B-chain comprising one amino acid that has been added, deleted, or substituted. In some embodiments of a modified therapeutic agent, the therapeutic agent further comprises a modified relaxin B-chain comprising two amino acid have been added, deleted, substituted, or a combination thereof. In some embodiments of a modified therapeutic agent, the therapeutic agent further comprises a modified relaxin B-chain comprising three amino acid have been added, deleted, substituted, or a combination thereof. In some embodiments of a modified therapeutic agent, the therapeutic agent further comprises a modified relaxin B-chain comprising four amino acid have been added, deleted, substituted, or a combination thereof. In some embodiments of a modified therapeutic agent, the therapeutic agent further comprises a modified relaxin B-chain comprising five amino acid have been added, deleted, substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a relaxin A-chain amino acid sequence selected from the group consisting of SEQ ID NO: 54, 55, 59 and 60; and
(ii) a half-life extending moiety;
   wherein the TA is covalently attached to the half-life extending moiety via a cysteine residue of the TA.

In some embodiments of a modified therapeutic agent, the cysteine residue is in the relaxin A-chain amino acid sequence. In some embodiments of a modified therapeutic agent, the cysteine residue is not in the relaxin A-chain amino acid sequence. In some embodiments of a modified therapeutic agent, the cysteine residue is at the 2 position of the relaxin A-chain amino acid sequence. In some embodiments of a modified therapeutic agent, the cysteine residue is at the 13 position of the relaxin A-chain amino acid sequence. In some embodiments of a modified therapeutic agent, the therapeutic agent comprises the relaxin A-chain amino acid sequence SEQ ID NO: 54. In some embodiments of a modified therapeutic agent, the therapeutic agent comprises the relaxin A-chain amino acid sequence SEQ ID NO: 55. In some embodiments of a modified therapeutic agent, the therapeutic agent comprises the relaxin A-chain amino acid sequence SEQ ID NO: 59. In some embodiments of a modified therapeutic agent, the therapeutic agent comprises the relaxin A-chain amino acid sequence SEQ ID NO: 60. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, substituted, or a combination thereof. In some embodiments of a modified therapeutic agent, the therapeutic agent further comprises a modified relaxin B-chain comprising one amino acid that has been added, deleted, or substituted. In some embodiments of a modified therapeutic agent, the therapeutic agent further comprises a modified relaxin B-chain comprising two amino acid have been added, deleted, substituted, or a combination thereof. In some embodiments of a modified therapeutic agent, the therapeutic agent further comprises a modified relaxin B-chain comprising three amino acid have been added, deleted, substituted, or a combination thereof. In some embodiments of a modified therapeutic agent, the therapeutic agent further comprises a modified relaxin B-chain comprising four amino acid have been added, deleted, substituted, or a combination thereof. In some embodiments of a modified therapeutic agent, the therapeutic agent further comprises a modified relaxin B-chain comprising five amino acid have been added, deleted, substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a relaxin A-chain amino acid sequence that is at least 50% homologous to an amino acid sequence selected from the group consisting of SEQ ID NO: 54, 55, 59 and 60; and
(ii) a half-life extending moiety;
   wherein the TA is covalently attached to the half-life extending moiety via a cysteine residue of the TA.

In some embodiments of a modified therapeutic agent, the relaxin A-chain amino acid sequence is at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or 96% homologous to an amino acid sequence selected from the group consisting of SEQ ID NO: 54, 55, 59 and 60. In some embodiments of a modified therapeutic agent, the relaxin A-chain amino acid sequence is at least 80% homologous to an amino acid sequence selected from the group consisting of SEQ ID NO: 54, 55, 59 and 60. In some embodiments of a modified therapeutic agent, the relaxin A-chain amino acid sequence is at least 90% homologous to an amino acid sequence selected from the group consisting of SEQ ID NO: 54, 55, 59 and 60. In some embodiments of a modified therapeutic agent, the cysteine residue is in the relaxin A-chain amino acid sequence. In some embodiments of a modified therapeutic agent, the cysteine residue is not in the relaxin A-chain amino acid sequence. In some embodiments of a modified therapeutic agent, the cysteine residue is at the 2 position of the relaxin A-chain amino acid sequence. In some embodiments of a modified therapeutic agent, the cysteine residue is at the 13 position of the relaxin A-chain amino acid sequence. In some embodiments of a modified therapeutic agent, the relaxin A-chain amino acid sequence is at least 50% homologous to the amino acid sequence SEQ ID NO: 54. In some embodiments of a modified therapeutic agent, the relaxin A-chain amino acid sequence is at least 50% homologous to the amino acid sequence SEQ ID NO: 55. In some embodiments of a modified therapeutic agent, the relaxin A-chain amino acid sequence is at least 50% homologous to the amino acid sequence SEQ ID NO: 59. In some embodiments of a modified therapeutic agent, the relaxin A-chain amino acid sequence is at least 50% homologous to the amino acid sequence SEQ ID NO: 60. In some embodiments of a modified therapeutic agent, the relaxin A-chain amino acid sequence is not SEQ ID NO: 41. In some embodiments of a modified therapeutic agent, the relaxin A-chain amino acid sequence is not SEQ ID NO: 42. In some embodiments of a modified therapeutic agent, the relaxin A-chain amino acid sequence is not SEQ ID NO: 43. In some embodiments of a modified therapeutic agent, the relaxin A-chain amino acid sequence is not SEQ ID NO: 44. In some embodiments of a modified therapeutic agent, the relaxin A-chain amino acid sequence is not SEQ ID NO: 45. In some embodiments of a modified therapeutic agent, the relaxin A-chain amino acid sequence is not SEQ ID NO: 46. In some embodiments of a modified therapeutic agent, the relaxin A-chain amino acid sequence is not SEQ ID NO: 47. In some embodiments of a modified therapeutic agent, the relaxin A-chain amino acid sequence is not SEQ ID NO: 53. In some embodiments of a modified therapeutic agent, the relaxin A-chain amino acid sequence is not SEQ ID NO: 55. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, substituted, or a combination thereof. In some embodiments of a modified therapeutic agent, the therapeutic agent further comprises a modified relaxin B-chain comprising one amino acid that has been added, deleted, or substituted. In some embodiments of a modified therapeutic agent, the therapeutic agent further comprises a modified relaxin B-chain comprising two amino acid have been added, deleted, substituted, or a combination thereof. In some embodiments of a modified therapeutic agent, the therapeutic agent further comprises a modified relaxin B-chain comprising three amino acid have been added, deleted, substituted, or a combination thereof. In some embodiments of a modified therapeutic agent, the therapeutic agent further comprises a modified relaxin B-chain comprising four amino acid have been added, deleted, substituted, or a combination thereof. In some embodiments of a modified therapeutic agent, the therapeutic agent further comprises a modified relaxin B-chain comprising five amino acid have been added, deleted, substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a relaxin A-chain amino acid sequence selected from the group consisting of SEQ ID NO: 54, 55, 59 and 60; wherein up to five amino acids have been added, deleted, substituted, or a combination thereof and
(ii) a half-life extending moiety;
   wherein the TA is covalently attached to the half-life extending moiety via a cysteine residue of the TA.

In some embodiments of a modified therapeutic agent, one amino acid has been added, deleted, or substituted from the relaxin A-chain amino acid sequence. In some embodiments of a modified therapeutic agent, two amino acid have been added, deleted, substituted, or a combination thereof from the relaxin A-chain amino acid sequence. In some embodiments of a modified therapeutic agent, three amino acid have been added, deleted, substituted, or a combination thereof from the relaxin A-chain amino acid sequence. In some embodiments of a modified therapeutic agent, four amino acid have been added, deleted, substituted, or a combination thereof from the relaxin A-chain amino acid sequence. In some embodiments of a modified therapeutic agent, five amino acid have been added, deleted, substituted, or a combination thereof from the relaxin A-chain amino acid sequence. In some embodiments of a modified therapeutic agent, the cysteine residue is in the relaxin A-chain amino acid sequence. In some embodiments of a modified therapeutic agent, the cysteine residue is not in the relaxin A-chain amino acid sequence. In some embodiments of a modified therapeutic agent, the cysteine residue is at the 2 position of the relaxin A-chain amino acid sequence. In some embodiments of a modified therapeutic agent, the cysteine residue is at the 13 position of the relaxin A-chain amino acid sequence. In some embodiments of a modified therapeutic agent, the relaxin A-chain amino acid sequence is SEQ ID NO: 54; wherein up to five amino acids have been added, deleted, substituted, or a combination thereof. In some embodiments of a modified therapeutic agent, the relaxin A-chain amino acid sequence is SEQ ID NO: 55; wherein up to five amino acids have been added, deleted, substituted, or a combination thereof. In some embodiments of a modified therapeutic agent, the relaxin A-chain amino acid sequence is SEQ ID NO: 59; wherein up to five amino acids have been added, deleted, substituted, or a combination thereof. In some embodiments of a modified therapeutic agent, the relaxin A-chain amino acid sequence is SEQ ID NO: 60; wherein up to five amino acids have been added, deleted, substituted, or a combination thereof. In some embodiments of a modified therapeutic agent, the relaxin A-chain amino acid sequence is not SEQ ID NO: 41. In some embodiments of a modified therapeutic agent, the relaxin A-chain amino acid sequence is not SEQ ID NO: 42. In some embodiments of a modified therapeutic agent, the relaxin A-chain amino acid sequence is not SEQ ID NO: 43. In some embodiments of a modified therapeutic agent, the relaxin A-chain amino acid sequence is not SEQ ID NO: 44. In some embodiments of a modified therapeutic agent, the relaxin A-chain amino acid sequence is not SEQ ID NO: 45. In some embodiments of a modified therapeutic agent, the relaxin A-chain amino acid sequence is not SEQ ID NO: 46. In some embodiments of a modified therapeutic agent, the relaxin A-chain amino acid sequence is not SEQ ID NO: 47. In some embodiments of a modified therapeutic agent, the relaxin A-chain amino acid sequence is not SEQ ID NO: 53. In some embodiments of a modified therapeutic agent, the relaxin A-chain amino acid sequence is not SEQ ID NO: 55. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, substituted, or a combination thereof. In some embodiments of a modified therapeutic agent, the therapeutic agent further comprises a modified relaxin B-chain comprising one amino acid that has been added, deleted, or substituted. In some embodiments of a modified therapeutic agent, the therapeutic agent further comprises a modified relaxin B-chain comprising two amino acid have been added, deleted, substituted, or a combination thereof. In some embodiments of a modified therapeutic agent, the therapeutic agent further comprises a modified relaxin B-chain comprising three amino acid have been added, deleted, substituted, or a combination thereof. In some embodiments of a modified therapeutic agent, the therapeutic agent further comprises a modified relaxin B-chain comprising four amino acid have been added, deleted, substituted, or a combination thereof. In some embodiments of a modified therapeutic agent, the therapeutic agent further comprises a modified relaxin B-chain comprising five amino acid have been added, deleted, substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

Disclosed herein are modified therapeutic agents comprising:
(i) a therapeutic agent (TA) comprising a portion of a relaxin A-chain amino acid sequence selected from the group consisting of SEQ ID NO: 54, 55, 59 and 60; and
(ii) a half-life extending moiety;
   wherein the TA is covalently attached to the half-life extending moiety via a cysteine residue of the TA.

In some embodiments of a modified therapeutic agent, the portion of the relaxin A-chain amino acid sequence comprises at least 20 consecutive amino acids of SEQ ID NO: 54, 55, 59 and 60. In some embodiments of a modified therapeutic agent, the portion of the relaxin A-chain amino acid sequence comprises at least 20, at least 19, at least 18, at least 17, at least 16, at least 15, at least 14, at least 13, at least 12, at least 11, at least 10, at least 9, at least 8, at least 7, at least 6, or at least 5 consecutive amino acids of SEQ ID NO: 54, 55, 59 and 60. In some embodiments of a modified therapeutic agent, the portion of the relaxin A-chain amino acid sequence comprises at least 15 consecutive amino acids of SEQ ID NO: 54, 55, 59 and 60. In some embodiments of a modified therapeutic agent, the portion of the relaxin A-chain amino acid sequence comprises at least 10 consecutive amino acids of SEQ ID NO: 54, 55, 59 and 60. In some embodiments of a modified therapeutic agent, the cysteine residue is in the portion of the relaxin A-chain amino acid sequence. In some embodiments of a modified therapeutic agent, the cysteine residue is not in the portion of the relaxin A-chain amino acid sequence. In some embodiments of a modified therapeutic agent, the cysteine residue is at the 2 position of the portion of the relaxin A-chain amino acid sequence. In some embodiments of a modified therapeutic agent, the cysteine residue is at the 2 position of the portion of the relaxin A-chain amino acid sequence. In some embodiments of a modified therapeutic agent, the therapeutic agent comprises a portion of the relaxin A-chain amino acid SEQ ID NO: 54. In some embodiments of a modified therapeutic agent, the therapeutic agent comprises a portion of the relaxin A-chain amino acid SEQ ID NO: 55. In some embodiments of a modified therapeutic agent, the therapeutic agent comprises a portion of the relaxin A-chain amino acid SEQ ID NO: 59. In some embodiments of a modified therapeutic agent, the therapeutic agent comprises a portion of the relaxin A-chain amino acid SEQ ID NO: 60. In some embodiments of a modified therapeutic agent, the relaxin A-chain amino acid sequence is not SEQ ID NO: 41. In some embodiments of a modified therapeutic agent, the relaxin A-chain amino acid sequence is not SEQ ID NO: 42. In some embodiments of a modified therapeutic agent, the relaxin A-chain amino acid sequence is not SEQ ID NO: 43. In some embodiments of a modified therapeutic agent, the relaxin A-chain amino acid sequence is not SEQ ID NO: 44. In some embodiments of a modified therapeutic agent, the relaxin A-chain amino acid sequence is not SEQ ID NO: 45. In some embodiments of a modified therapeutic agent, the relaxin A-chain amino acid sequence is not SEQ ID NO: 46. In some embodiments of a modified therapeutic agent, the relaxin A-chain amino acid sequence is not SEQ ID NO: 47. In some embodiments of a modified therapeutic agent, the relaxin A-chain amino acid sequence is not SEQ ID NO: 53. In some embodiments of a modified therapeutic agent, the relaxin A-chain amino acid sequence is not SEQ ID NO: 55. In some embodiments of a modified therapeutic agent, the therapeutic agent further comprises a relaxin B-chain. In some embodiments of a modified therapeutic agent, the therapeutic agent further comprises a modified relaxin B-chain wherein one or more amino acids have been added, deleted, substituted, or a combination thereof. In some embodiments of a modified therapeutic agent, the therapeutic agent further comprises a modified relaxin B-chain comprising one amino acid that has been added, deleted, or substituted. In some embodiments of a modified therapeutic agent, the therapeutic agent further comprises a modified relaxin B-chain comprising two amino acid have been added, deleted, substituted, or a combination thereof. In some embodiments of a modified therapeutic agent, the therapeutic agent further comprises a modified relaxin B-chain comprising three amino acid have been added, deleted, substituted, or a combination thereof. In some embodiments of a modified therapeutic agent, the therapeutic agent further comprises a modified relaxin B-chain comprising four amino acid have been added, deleted, substituted, or a combination thereof. In some embodiments of a modified therapeutic agent, the therapeutic agent further comprises a modified relaxin B-chain comprising five amino acid have been added, deleted, substituted, or a combination thereof. In some embodiments of a modified therapeutic agent, the therapeutic agent further comprises a relaxin B-chain amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence that is at least 50% homologous to an amino acid sequence selected from the group consisting of SEQ ID NO: 16, 40, 48-52, 56, 58, and 61. In some embodiments of a modified therapeutic agent, the therapeutic agent further comprises a relaxin B-chain amino acid sequence of SEQ ID NO: 48. In some embodiments of a modified therapeutic agent, the half-life of the modified therapeutic agent is longer than the half-life of the therapeutic agent alone.

In some embodiments, the modified therapeutic agent has the following formula:

TA-A¹-P¹-L Formula (A)

wherein:
TA is the therapeutic agent;

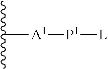

is the half-life extending moiety.

In some embodiments of a modified therapeutic agent,
A¹ is a chemical group linking TA and P¹ or L;
P¹ is a bond or -PEG-A²-; and
L is a lipid derivative selected from the group consisting of sterol derivatives, bile acid derivatives, vitamin E derivatives, fatty di-acid derivatives, fatty acid derivatives, fatty amide derivatives, fatty amine amine derivatives, and fatty alcohol derivatives; wherein the lipid derivative is linked to P¹ via an amide or an ether linkage.

In some embodiments of a modified therapeutic agent, the lipid derivative is selected from the group consisting of propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, myristic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid and hexatriacontanoic acid, malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, pentadecanedioic acid, hexadecanedioic acid, heptadecanedioic acid, octadecanedioic acid, and nonadecanedioic acid, myristoleic acid, palmitoleic acid, sapienic acid, oleic acid, elaidic acid, vaccenic acid, linoleic acid, linoelaidic acid, α-linolenic acid, arachidonic acid, eicosapentanoic acid, erucic acid, and docosahexaenoic acid.

In some embodiments of a modified therapeutic agent, the lipid derivative is selected from the group consisting of octanoic acid, nonanoic acid, decanoic acid, undecanoic acid, dodecanoic acid, tridecanoic acid, tetradecanoic acid, myristic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid and henatriacontanoic acid, malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, pentadecanedioic acid, hexadecanedioic acid, heptadecanedioic acid, octadecanedioic acid, and nonadecanedioic acid, myristoleic acid, palmitoleic acid, sapienic acid, oleic acid, elaidic acid, vaccenic acid, linoleic acid, linoelaidic acid, α-linolenic acid, arachidonic acid, eicosapentanoic acid, erucic acid, and docosahexaenoic acid.

In some embodiments of a modified therapeutic agent, the lipid derivative is selected from the group consisting of cholesterol, 7-OH cholesterol, 7,25-dihydroxycholesterol, cholic acid, chenodeoxycholic acid, lithocholic acid, deoxycholic acid, glycocholic acid, glycodeoxycholic acid, glycolithocholic acid, and glycochenodeoxycholic acid.

In some embodiments of a modified therapeutic agent, the lipid derivative is selected from the group consisting of α-tocopherol, β-tocopherol, γ-tocopherol, δ-tocopherol, α-tocotrienol, β-tocotrienol, γ-tocotrienol and δ-tocotrienol.

In some embodiments of a modified therapeutic agent, a sulfur atom of the cysteine residue of the TA is connected to A¹ via a chemical bond.

In some embodiments of a modified therapeutic agent of Formula (A),
A¹ is selected from

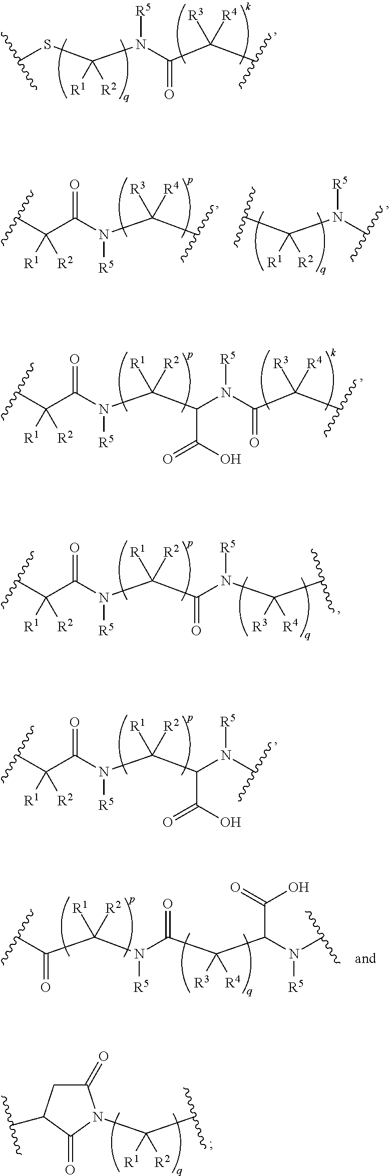

PEG is selected from

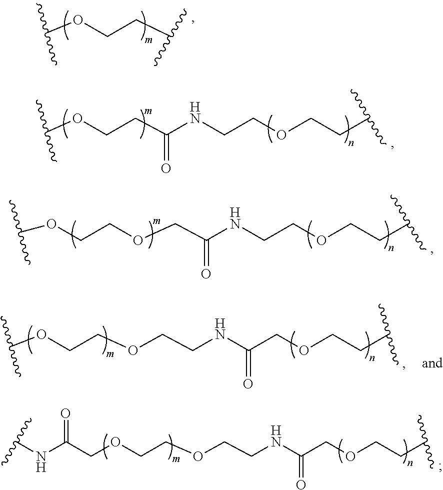

A² is selected from a bond,

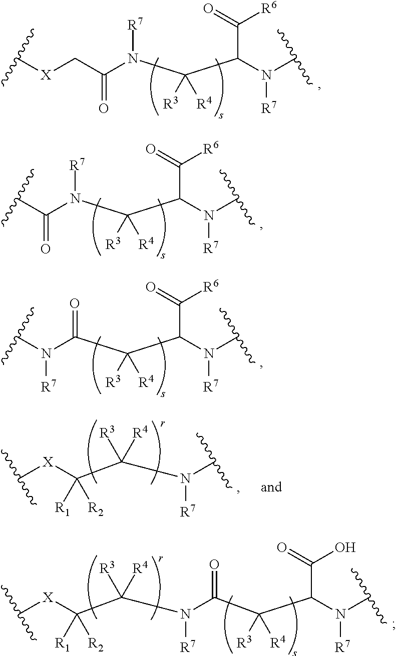

L is an acid moiety selected from tetradecanoic acid, octadecanedioic acid, myristic acid, stearic acid, docosahexaenoic acid, lithocholic acid, cholic acid, and palmitic acid; wherein the acid moiety is linked to P′ via an amide linkage;
X is a bond, —N(R⁵)—, —S—, or —O—;
each R¹, R², R³, and R⁴ is independently H, halo, —CN, —SR⁵, alkyl, cycloalkyl, haloalkyl, —NR⁵R⁵, or —OR⁵;
each R⁵ is independently H, alkyl, haloalkyl, arylalkyl, or heteroalkyl;
R⁶ is OH or —NR⁵R⁵;
each R⁷ is independently H, alkyl, haloalkyl, arylalkyl, or heteroalkyl;
m and n are independently 1, 2, 3, 4, 5, 6, 7, 8, 9, 10, 11, 12, 13, 14, 15, 16, 17, 18, 19, or 20;
r is 1, 2, 3, 4, 5, 6, 7, 8, 9, or 10;
s is 1, 2, 3, 4, or 5;
k is 1, 2, 3, 4, 5, 6, 7, 8, 9, or 10;
p is 2, 3, 4, 5, 6, 7, 8, 9, or 10; and
q is 2, 3, 4, 5, 6, 7, 8, 9, or 10.

In some embodiments of a modified therapeutic agent, A¹ is

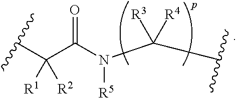

In some embodiments of a modified therapeutic agent, PEG is

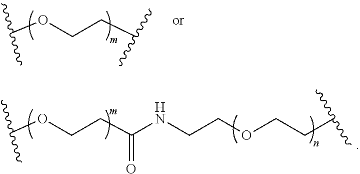

In some embodiments of a modified therapeutic agent, A² is

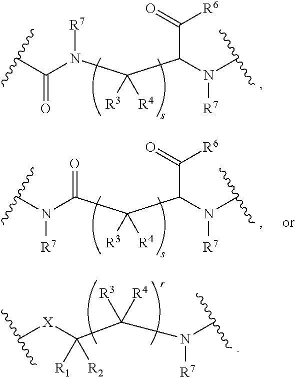

In some embodiments of a modified therapeutic agent, A¹ is

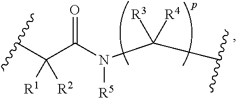

### PEG is

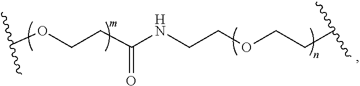

### and A² is

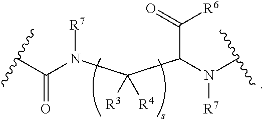

In some embodiments of a modified therapeutic agent, A¹ is

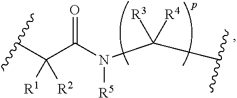

### PEG is

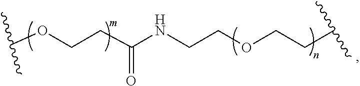

### A² is

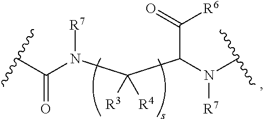

X is —O—; each R¹, R², R³, R⁴, R⁵, R⁶, and R⁷ is H; m is 1 or 2; p is 2, n is 1; and s is 2, 3, or 4.

In some embodiments of a modified therapeutic agent, A¹ is

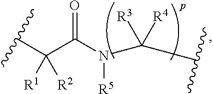

### PEG is

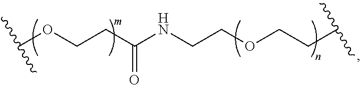

### and A² is

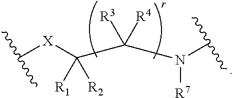

In some embodiments of a modified therapeutic agent, A¹ is

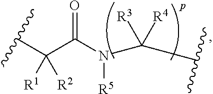

### PEG is

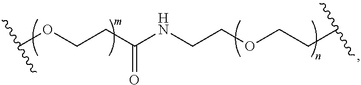

### A² is

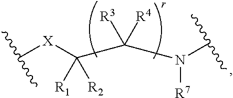

X is —O—; each R¹, R², R³, R⁴, R⁵, R⁶, and R⁷ is H; m is 1 or 2; p is 2; r is 1 or 2; and n is 1.

In some embodiments of a modified therapeutic agent, A¹ is

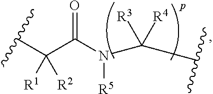

### PEG is

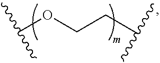

### and A² is

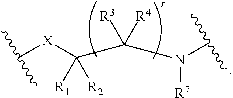

In some embodiments of a modified therapeutic agent, A¹ is

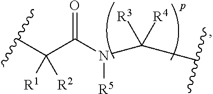

### PEG is

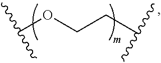

### A² is

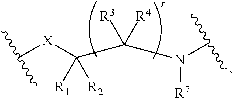

X is —O—; each R¹, R², R³, R⁴, R⁵, R⁶, and R⁷ is H; m is 1 or 2; p is 2; n is 1; r is 1 or 2; and s is 2, 3, or 4.

In some embodiments of a modified therapeutic agent, A¹ is

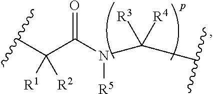

### PEG is

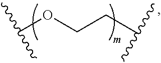

### and A² is

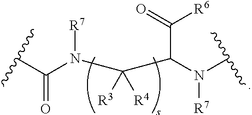

In some embodiments of a modified therapeutic agent, A¹ is

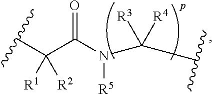

### PEG is

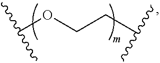

### A² is

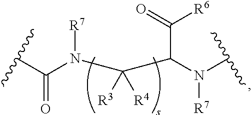

each R¹, R², R³, R⁴, R⁵, R⁶, and R⁷ is H; m is 5, 6, 7, or 8; p is 2; and s is 2, 3, or 4.

In some embodiments of a modified therapeutic agent, each R¹, R², R³, and R⁴ is H. In some embodiments of a modified therapeutic agent, each R⁵ is independently H. In some embodiments of a modified therapeutic agent, each R⁶ is OH or NH₂. In some embodiments of a modified therapeutic agent, each R⁶ is OH. In some embodiments of a modified therapeutic agent, each R⁷ is H. In some embodiments of a modified therapeutic agent, m is 1, 2, 3, 4, 5, 6, 7, 8, or 9. In some embodiments of a modified therapeutic agent, m is 1. In some embodiments of a modified therapeutic agent, m is 2. In some embodiments of a modified therapeutic agent, m is 5. In some embodiments of a modified therapeutic agent, m is 8. In some embodiments of a modified therapeutic agent, n is 1, 2, 3, 4, or 5. In some embodiments of a modified therapeutic agent, n is 1. In some embodiments of a modified therapeutic agent, r is 1, 2, or 3. In some embodiments of a modified therapeutic agent, r is 1. In some embodiments of a modified therapeutic agent, s is 2 or 4. In some embodiments of a modified therapeutic agent, s is 2. In some embodiments of a modified therapeutic agent, s is 4. In some embodiments of a modified therapeutic agent, p is 2. In some embodiments of a modified therapeutic agent, X is —O—.

In some embodiments of a modified therapeutic agent, L is an acid moiety selected from tetradecanoic acid, octadecanedioic acid, myristic acid, stearic acid, docosahexaenoic acid, lithocholic acid, cholic acid, and palmitic acid; P¹ is -PEG-A²-; and wherein the acid moiety is linked to A² via an amide linkage. In some embodiments, the amide linkage is formed between one amino moiety of A² and one acid moiety of L. In some embodiments, the acid derivative is linked to the rest of the molecule according to the following formula:

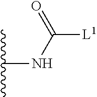

wherein L¹ is a portion of tetradecanoic acid, octadecanedioic acid, myristic acid, stearic acid, docosahexaenoic acid, lithocholic acid, cholic acid, or palmitic acid and NH is part of A². In some embodiments, L¹ is

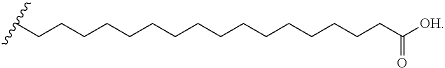 [SMILES: *CCCCCCCCCCCCCCCCC(=O)O]

In some embodiments, L¹ is

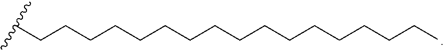 [SMILES: *CCCCCCCCCCCCCCCCC]

In some embodiments, L¹ is

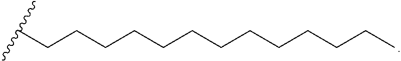 [SMILES: *CCCCCCCCCCCCC]

In some embodiments the modified therapeutic agent is selected from the group consisting of:

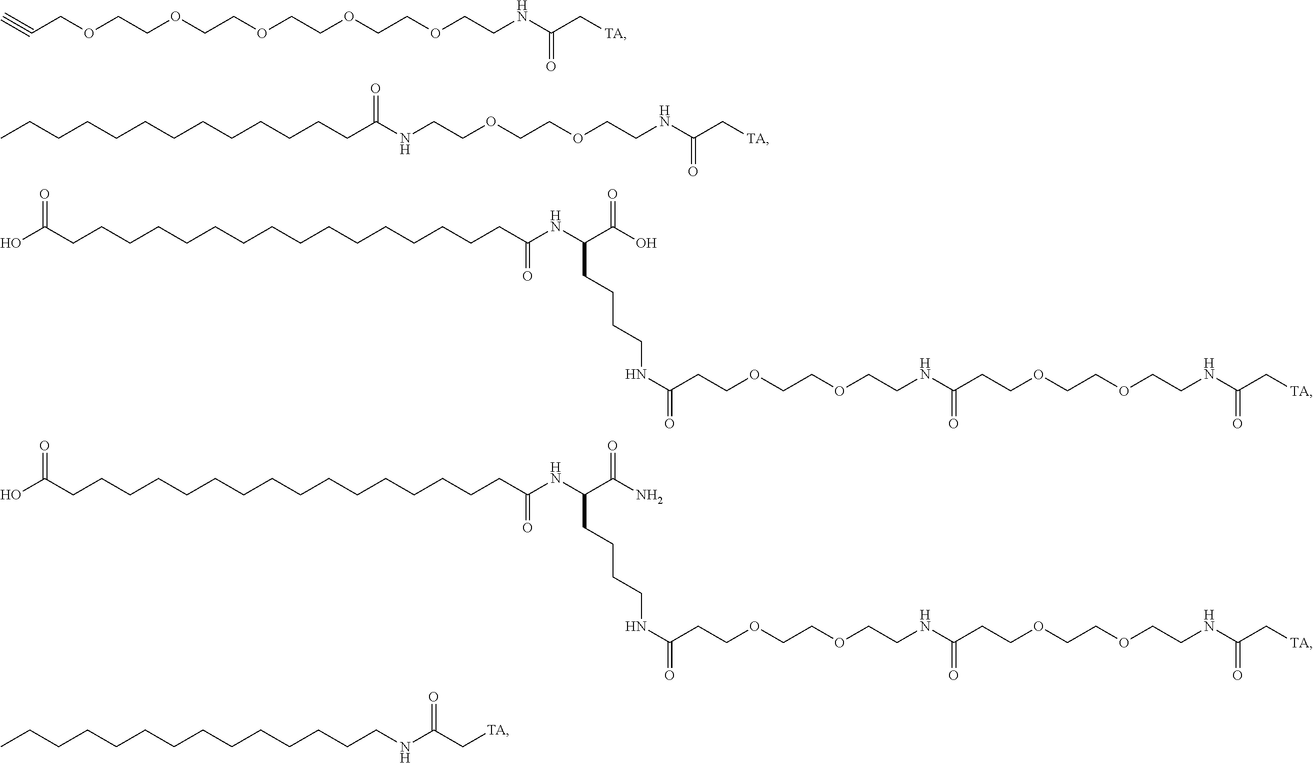

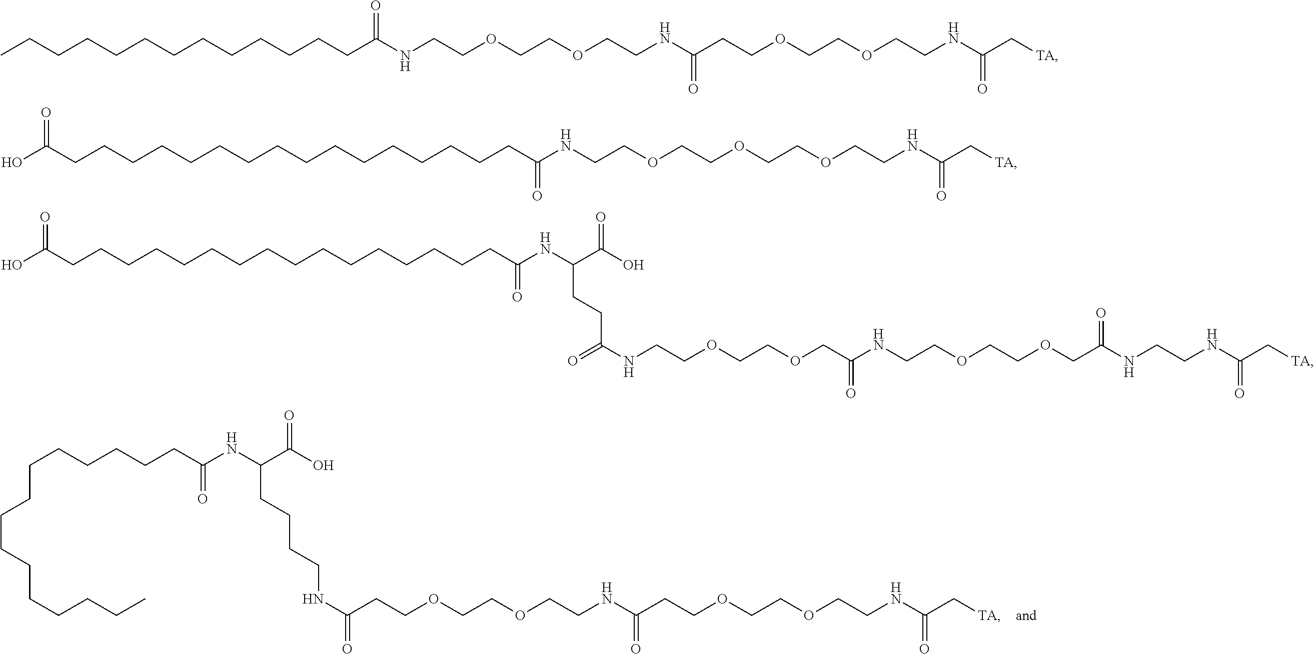

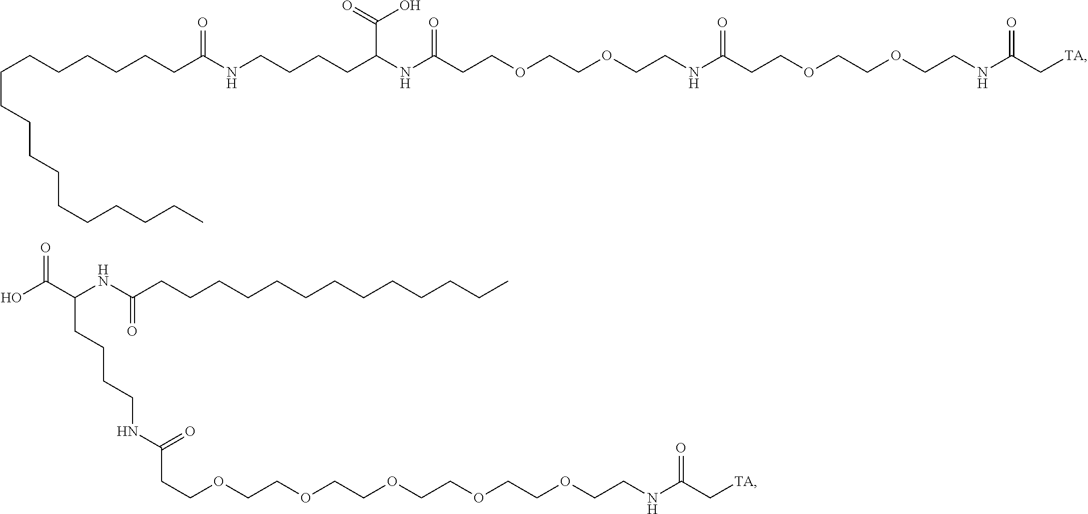

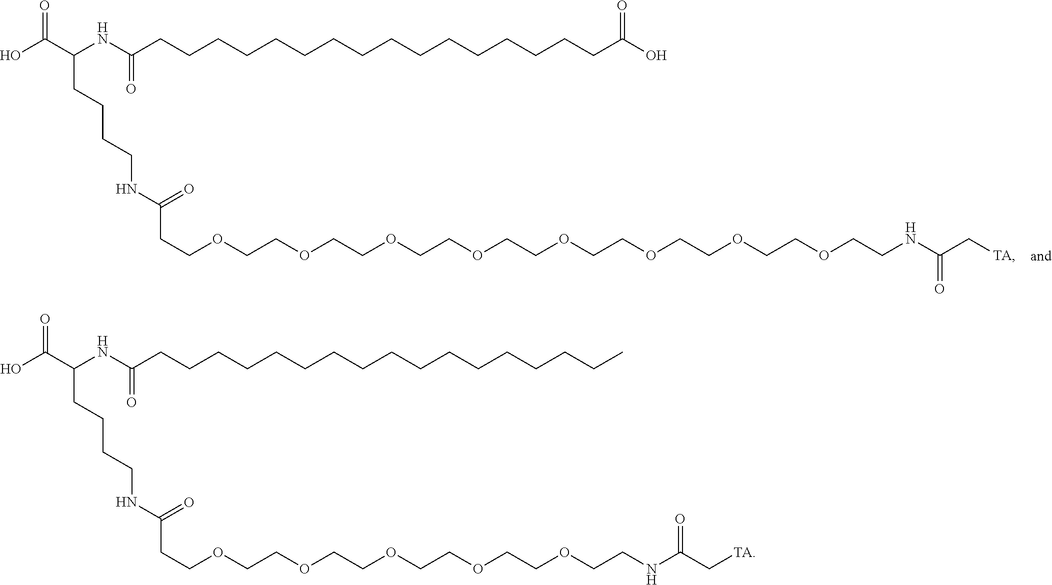

In some embodiments the modified therapeutic agent is

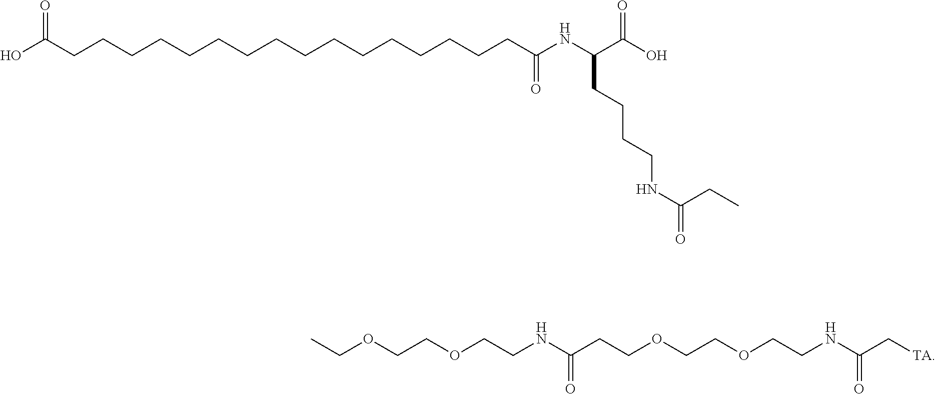

Disclosed herein are methods of producing an modified therapeutic agent of Formula (A), the method comprising reacting the cysteine residue of TA with A³-P¹-X, wherein A³ is a reactive precursor to form A¹. In some embodiments, A³ is a haloacetamide, maleimide, benzyl halide, or pyridyl disulfide. In further embodiments, A³ is a haloacetamide. In still further embodiments, A³ is a bromoacetamide.

Also disclosed herein are modified therapeutic agents comprising a therapeutic agent (TA) and one or more half-life extending moieties, wherein the TA is attached to the one or more half-life extending moieties. The therapeutic agent may be a peptide. The TA may be covalently attached to each of the one or more half-life extending moieties. The TA may be attached to the one or more half-life extending moieties via an amino acid residue on the therapeutic agent. The amino acid residue may be a cysteine or lysine. The half-life of the modified therapeutic agent may be longer than the half-life of the therapeutic agent alone. A half-life extending moiety may comprise a lipid, a polyglycol region, or a combination thereof. Non-limiting examples of modified therapeutic agents include lipid conjugates (LCs).

Disclosed herein are modified therapeutic agents comprising a therapeutic agent (TA) and one or more half-life extending moieties, wherein the TA is attached to the one or more half-life extending moieties. The therapeutic agent may be a peptide. The TA may comprise at least one isosteric disulfide mimetic. The isosteric disulfide mimetic may comprise a heteroaryl group. The TA may be covalently attached to each of the one or more half-life extending moieties. The TA may be attached to the one or more half-life extending moieties via an amino acid residue on the therapeutic agent. The amino acid residue may be a cysteine or lysine. The half-life of the modified therapeutic agent may be longer than the half-life of the therapeutic agent alone. A half-life extending moiety may comprise a lipid, a polyglycol region, or a combination thereof. The half-life extending moiety may further comprise a heteroaryl or heterocyclyl group. A half-life extending moiety may be attached to the sulfur atom of a cysteine residue on the therapeutic agent. Non-limiting examples of modified therapeutic agents include lipid conjugates (LCs).

Disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the one or more half-life extending moieties comprises a heteroaryl or heterocyclyl group; and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a lipid and a heteroaryl or heterocyclyl group; and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a lipid and a heteroaryl or heterocyclyl group; the peptide is selected from relaxin, H1 relaxin, H2 relaxin, H3 relaxin, human INSL3, human INSL4, human INSL6, human IGF1, human IGFII, human insulin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, peptide-based toxin, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a lipid and a heteroaryl or heterocyclyl group; the peptide is selected from relaxin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, Toxin-550, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a lipid and a heteroaryl or heterocyclyl group; the peptide is relaxin or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a lipid and a heteroaryl or heterocyclyl group; the therapeutic agent is encoded by an amino acid sequence comprising at least a portion of a polypeptide sequence selected from the group consisting of SEQ ID NO: 10-61; and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a lipid selected from sterols, sterol derivatives, bile acids, vitamin E derivatives, fatty di-acids, fatty acids, fatty amides, fatty amines, and fatty alcohols, and derivatives thereof; and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via amino acid residue on the peptide; each of the half-life extending moieties comprises a heteroaryl or heterocyclyl group, and a lipid selected from sterols, sterol derivatives, bile acids, vitamin E derivatives, fatty di-acids, fatty acids, fatty amides, fatty amines, and fatty alcohols, and derivatives thereof; the peptide is selected from relaxin, H1 relaxin, H2 relaxin, H3 relaxin, human INSL3, human INSL4, human INSL6, human IGF1, human IGFII, human insulin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, peptide-based toxin, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a lipid selected from sterols, sterol derivatives, bile acids, vitamin E derivatives, fatty di-acids, fatty acids, fatty amides, fatty amines, and fatty alcohols, and derivatives thereof; the peptide is selected from relaxin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, Toxin-550, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a lipid selected from sterols, sterol derivatives, bile acids, vitamin E derivatives, fatty di-acids, fatty acids, fatty amides, fatty amines, and fatty alcohols, and derivatives thereof; the peptide is relaxin or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via amino acid residue on the peptide; each of the half-life extending moieties comprises a heteroaryl or heterocyclyl group, and a polyglycol region; and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a polyglycol region; the peptide is selected from relaxin, H1 relaxin, H2 relaxin, H3 relaxin, human INSL3, human INSL4, human INSL6, human IGF1, human IGFII, human insulin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, peptide-based toxin, Moka, and VM-24, or a derivative thereof, the derivative being a peptide comprising one or more amino acid additions, deletions, or substitutions, or a combination thereof and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a polyglycol region; the peptide is selected from relaxin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, Toxin-550, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a polyglycol region; the peptide is relaxin or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, and a polyglycol region; the therapeutic agent is encoded by an amino acid sequence comprising at least a portion of a polypeptide sequence selected from the group consisting of SEQ ID NO: 10-61; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, a lipid, and a polyglycol region; and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, a polyglycol region, and a lipid selected from sterols, sterol derivatives, bile acids, vitamin E derivatives, fatty di-acids, fatty acids, fatty amides, fatty amines, and fatty alcohols, and derivatives thereof and the half-life of the modified therapeutic agent is longer than the half-life of the peptide alone. The peptide may comprise one or more amino acid additions, deletions, substitutions, or a combination thereof. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, a polyglycol region, and a lipid selected from sterols, sterol derivatives, bile acids, vitamin E derivatives, fatty di-acids, fatty acids, fatty amides, fatty amines, and fatty alcohols, and derivatives thereof; the peptide is selected from relaxin, H1 relaxin, H2 relaxin, H3 relaxin, human INSL3, human INSL4, human INSL6, human IGF1, human IGFII, human insulin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, peptide-based toxin, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via amino acid residue on the peptide; each of the half-life extending moieties comprises a heteroaryl or heterocyclyl group, a polyglycol region, and a lipid selected from sterols, sterol derivatives, bile acids, vitamin E derivatives, fatty di-acids, fatty acids, fatty amides, fatty amines, and fatty alcohols, and derivatives thereof; the peptide is selected from relaxin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, Toxin-550, Moka, and VM-24, or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be an amino acid addition or substitution on a wild-type peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the half-life extending moieties comprises a heteroaryl or heterocyclyl group, a polyglycol region, and a lipid selected from sterols, sterol derivatives, bile acids, vitamin E derivatives, fatty di-acids, fatty acids, fatty amides, fatty amines, and fatty alcohols, and derivatives thereof; the peptide is relaxin or a derivative thereof, the derivative being a peptide comprising one or more amino acid additions, deletions, or substitutions, or a combination thereof; and the half-life of the modified therapeutic agent is longer than the half-life of the peptide alone. The amino acid residue may be an amino acid addition or substitution on the peptide. The amino acid residue may be a cysteine or a lysine. The amino acid residue may be a cysteine. Each of the one or more half-life extending moieties may comprise a heteroaryl group. Each of the one or more half-life extending moieties may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole. The modified therapeutic agent may be an LC.

The modified therapeutic agents disclosed herein may have the structure:

TA-A¹-P¹-X Formula (I)

wherein:
TA is the therapeutic agent with an amino acid residue, wherein the amino acid residue is connected to A¹;
A¹ is a chemical group linking TA and P¹;
P¹ comprises a heteroaryl or heterocyclyl group; and
X is a half-life extending moiety.

X of Formula (I) may comprise a lipid.

The amino acid of the TA of an modified therapeutic agent of Formula (I) may be a cysteine or lysine. The sulfur or nitrogen atom of an amino acid residue of the TA of an modified therapeutic agent of Formula (I) may be connected to A¹ via a chemical bond.

The amino acid of the TA of an modified therapeutic agent of Formula (I) may be a cysteine. The sulfur atom of a cysteine residue of the TA of an modified therapeutic agent of Formula (I) may be connected to A¹ via a chemical bond. The amino acid of the TA of an modified therapeutic agent of Formula (I) may be a lysine. The nitrogen atom of a cysteine residue of the TA of an modified therapeutic agent of Formula (I) may be connected to A¹ via a chemical bond.

P¹ of an modified therapeutic agent of Formula (I) may be -PEG-A²-; wherein PEG is a chemical group comprising one or more polyethyleneglycol subunits and A² comprises a heteroaryl or heterocyclyl group and is a chemical group linking PEG and X. A² may comprise a heteroaryl group. A² may comprise a heterocyclyl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole ring.

The half-life extending moiety of Formula (I) may comprise a lipid. The lipid may be selected from the group consisting of propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, myristic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid and hexatriacontanoic acid. The lipid may be selected from the group consisting of malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, pentadecanedioic acid, hexadecanedioic acid, heptadecanedioic acid, octadecanedioic acid, and nonadecanedioic acid. The lipid may be selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentanoic acid, erucic acid, docosahexaenoic acid. The lipid may be selected from the group consisting of cholesterol, 7-OH cholesterol, 7,25-dihydroxycholesterol, cholic acid, chenodeoxycholic acid, lithocholic acid, deoxycholic acid, glycocholic acid, glycodeoxycholic acid, glycolithocholic acid, and glycochenodeoxycholic acid. The one or more lipids of an modified therapeutic agent of Formula (I) may be selected from the group consisting of cholic acid, chenodeoxycholic acid, lithocholic acid, deoxycholic acid, glycocholic acid, glycodeoxycholic acid, glycolithocholic acid, and glycochenodeoxycholic acid. The one or more lipids of an modified therapeutic agent of Formula (I) may be selected from the group consisting of α-tocopherol, β-tocopherol, γ-tocopherol, δ-tocopherol, α-tocotrienol, β-tocotrienol, γ-tocotrienol and δ-tocotrienol. The one or more lipids of an modified therapeutic agent of Formula (I) may be selected from the group consisting of octadecanedioic acid, tetradecylamine, myristic acid, docosahexaenoic acid, lithocholic acid ester, cholic acid and palmitic acid.

The PEG of an modified therapeutic agent of Formula (I) may be selected from:

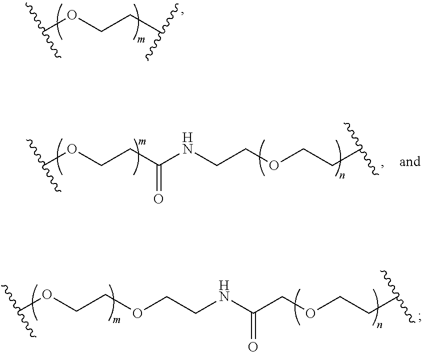

wherein
m and n are independently 1, 2, 3, 4, 5, 6, 7, 8, 9, 10, 11, 12, 13, 14, 15, 16, 17, 18, 19, or 20.

In some embodiments of an modified therapeutic agent of Formula (I) disclosed herein,
A¹ is selected from

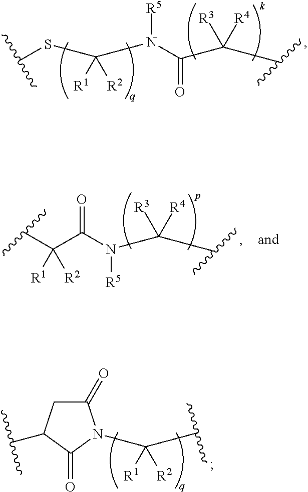

and
each R¹, R², R³, and R⁴ is independently selected from H, halo, CN, —SR⁵, alkyl, cycloalkyl, haloalkyl, —NR⁵R⁵, —NC(O)R⁵, —NC(O)OR⁵, and —OR⁵;
each R⁵ is independently H, alkyl, haloalkyl, arylalkyl, (cycloalkyl)alkyl, or heteroalkyl;
p is 2, 3, 4, 5, 6, 7, 8, 9, or 10; and
q is 2, 3, 4, 5, 6, 7, 8, 9, or 10.

The P¹ of an modified therapeutic agent of Formula (I) may comprise polyglycol. The polyglycol may be selected from polyethylene glycol, polypropylene glycol, polybutylene glycol, or a combination thereof. The polyglycol may be polyethylene glycol. The polyglycol may be polypropylene glycol. The polyglycol may be polybutylene glycol.

Disclosed herein are methods of producing an modified therapeutic agent of Formula (I), the method comprising reacting the cysteine residue of TA with A³-P¹-X, wherein A³ is a reactive precursor to form A¹. In some embodiments, A³ is a haloacetamide, maleimide, benzyl halide, or pyridyl disulfide. In further embodiments, A³ is a haloacetamide. In still further embodiments, A³ is a bromoacetamide.

Further disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; the peptide comprises at least one isosteric disulfide mimetic; and the half-life of the modified therapeutic agent is longer than the half-life of the peptide alone. Each of the one or more half-life extending moieties may comprise a lipid, a polyglycol region, or a combination thereof. Each of the one or more half-life extending moieties may comprise a lipid. Each of the one or more half-life extending moieties may comprise a polyglycol region. Each of the one or more half-life extending moieties may comprise a lipid and a polyglycol region. The lipid may be selected from the group consisting of sterols, sterol derivatives, bile acids, vitamin E derivatives, fatty di-acids, fatty acids, fatty amides, fatty amines, and fatty alcohols, and derivatives thereof. The polyglycol region may comprise one or more polyethylene glycol units, polypropylene glycol units, or polybutylene glycol units, or a combination thereof. The polyglycol region may comprise one thousand or more polyethylene glycol units, polypropylene glycol units, or polybutylene glycol units, or a combination thereof. The isosteric disulfide mimetic may comprise a heteroaryl group. The heteroaryl group may comprise at least one nitrogen. The heteroaryl group may be a 5-membered ring comprising at least one nitrogen. The peptide may comprise one or more amino acid additions, deletions, or substitutions, or a combination thereof. The amino acid residue, to which the half-life extending moiety is attached, may be located on the N-terminus or C-terminus of the peptide. The amino acid residue, to which the half-life extending moiety is attached, may be located on a non-terminus position of the peptide. The amino acid residue, to which the half-life extending moiety is attached, may be an amino acid addition or substitution on the peptide. The peptide may be a peptidyl toxin. The peptide may be selected from Toxin-550, Moka, and VM-24, or a derivative thereof, the derivative being a peptide comprising one or more amino acid additions, deletions, or substitutions, or a combination thereof. The peptide may comprise an amino acid sequence comprising at least a portion of a polypeptide sequence selected from the group consisting of selected from the group consisting of SEQ ID NO: 22, 23, and 26-31. The peptide may comprise an amino acid sequence comprising 10 or more amino acids based on or derived from a polypeptide sequence selected from the group consisting of selected from the group consisting of SEQ ID NO: 22, 23, and 26-31. The peptide may comprise an amino acid sequence that is at least 50% homologous to an amino acid sequence selected from the group comprising SEQ ID NO: 22, 23, and 26-31. The peptide may comprise an amino acid sequence that is at least 80% homologous to an amino acid sequence selected from the group comprising SEQ ID NO: 22, 23, and 26-31.

### Half-Life Extending Moieties

Disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; each of the one or more half-life extending moieties comprises a heteroaryl or heterocyclyl group; and the half-life of the modified therapeutic agent is longer than the half-life of the peptide alone. Also disclosed herein are modified therapeutic agents comprising a therapeutic agent and one or more half-life extending moieties, wherein the therapeutic agent is a peptide that is covalently attached to each of the one or more half-life extending moieties via an amino acid residue on the peptide; the peptide comprises at least one isosteric disulfide mimetic; and the half-life of the modified therapeutic agent is longer than the half-life of the peptide alone.

The one or more half-life extending moieties may comprise a lipid, a polyglycol region, or a combination thereof. The one or more half-life extending moieties may comprise an extended recombinant polypeptide (XTEN).

The polyglycol region may comprise one or more polyethylene glycol units, polypropylene glycol units, or polybutylene glycol units, or a combination thereof. The polyglycol region may comprise one or more polyethylene glycol units. The polyglycol region may comprise one or more polypropylene glycol units. The polyglycol region may comprise one or more polybutylene glycol units.

The polyglycol region may comprise 10, 20, 30, 40, 50, 60, 70, 80, 90, 100, or more polyethylene glycol units, polypropylene glycol units, or polybutylene glycol units, or a combination thereof. The polyglycol region may comprise 10, 20, 30, 40, 50, 60, 70, 80, 90, 100, or more polyethylene glycol units. The polyglycol region may comprise 10, 20, 30, 40, 50, 60, 70, 80, 90, 100, or more polypropylene glycol units. The polyglycol region may comprise 10, 20, 30, 40, 50, 60, 70, 80, 90, 100, or more polybutylene glycol units.

The polyglycol region may comprise 100, 200, 300, 400, 500, 600, 700, 800, 900, 1000, or more polyethylene glycol units, polypropylene glycol units, or polybutylene glycol units, or a combination thereof. The polyglycol region may comprise 100, 200, 300, 400, 500, 600, 700, 800, 900, 1000, or more polyethylene glycol units. The polyglycol region may comprise 100, 200, 300, 400, 500, 600, 700, 800, 900, 1000, or more polypropylene glycol units. The polyglycol region may comprise 100, 200, 300, 400, 500, 600, 700, 800, 900, 1000, or more polybutylene glycol units.

The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polyethylene glycol units, polypropylene glycol units, or polybutylene glycol units, or a combination thereof. The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polyethylene glycol units. The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polypropylene glycol units. The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polybutylene glycol units.

The polyglycol region may comprise a molecular weight of 500-50,000 daltons. The polyglycol region may comprise a molecular weight of 500-40,000 daltons. The polyglycol region may comprise a molecular weight of 500-30,000 daltons. The polyglycol region may comprise a molecular weight of 500-20,000 daltons. The polyglycol region may comprise a molecular weight of 500, 600, 700, 800, 900, 1000, 2000, 3000, 4000, 5000, 6000, 7000, 8000, 9000, 10000, 15000, 20000, 25000, 30000, 35000, 40000, or 45000 daltons or more, including increments therein.

The half-life extending moieties may be attached to the TA via a cysteine residue on the TA. The attachment may be via chemical attachment through the sulfur atom of a cysteine residue on the TA. The half-life extending moieties may comprise a linker which is directly attached to the sulfur atom of a cysteine residue on the TA. The half-life extending moieties may comprise a linker which is covalently attached to the sulfur atom of a cysteine residue on the TA.

The half-life extending moieties may be attached to the TA via a lysine residue on the TA. The attachment may be via chemical attachment through the nitrogen atom of a lysine residue on the TA. The half-life extending moieties may comprise a linker which is directly attached to the nitrogen atom of a lysine residue on the TA. The half-life extending moieties may comprise a linker which is covalently attached to the nitrogen atom of a lysine residue on the TA.

### Lipid Conjugate (LC)

Disclosed herein are lipid conjugates (LCs) comprising one or more lipids attached to one or more therapeutic agents (TAs). The lipid conjugate may comprise one or more lipids attached to one TA. The lipid conjugate may further comprise a hydrophilic connector between the one or more TAs and the one or more lipids. The lipid conjugate may further comprise one or more polyethyleneglycol subunits. The lipid conjugate may further comprise a heteroaryl or heterocyclyl group. The one or more lipids may be pegylated.

The LCs disclosed herein may have the structure:

TA-A¹-P¹-L Formula (II)

wherein:
TA is the therapeutic agent;
A¹ is a chemical group linking TA and P¹ or L;
P¹ comprises a heteroaryl or heterocyclyl group; and
L is the lipid.

A sulfur or nitrogen atom of an amino acid residue of TA may be connected to A¹ via a chemical bond in an LC of Formula (II). The A¹ of an LC of Formula (II) may be selected from

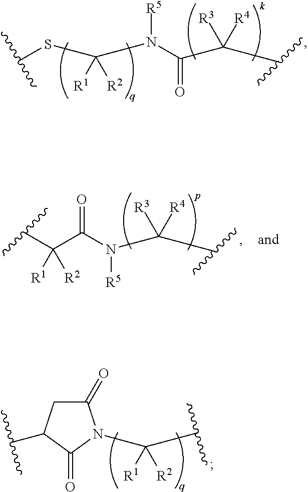

and
each R², R³, and R⁴ is independently selected from H, halo, CN, —SR⁵, alkyl, cycloalkyl, haloalkyl, —NR⁵R⁵, —NC(O)R⁵, —NC(O)OR⁵, and —OR⁵;
each R⁵ is independently H, alkyl, haloalkyl, arylalkyl, (cycloalkyl)alkyl, or heteroalkyl;
k is 1, 2, 3, 4, 5, 6, 7, 8, 9, or 10;
p is 2, 3, 4, 5, 6, 7, 8, 9, or 10; and
q is 2, 3, 4, 5, 6, 7, 8, 9, or 10.

The P¹ of an LC of Formula (II) may further comprise polyglycol. The polyglycol may be selected from polyethylene glycol, polypropylene glycol, polybutylene glycol, or a combination thereof. The polyglycol may be polyethylene glycol. The polyglycol may be polypropylene glycol. The polyglycol may be polybutylene glycol.

The P¹ of an LC of Formula (II) may be -PEG-A²-; wherein PEG is a chemical group comprising one or more polyethyleneglycol subunits; and A² comprises a heteroaryl or heterocyclyl group and connects PEG and L.

The PEG of an LC of Formula (II) may be selected from

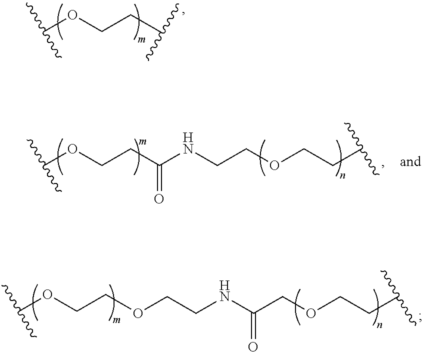

wherein m and n are independently 1, 2, 3, 4, 5, 6, 7, 8, 9, 10, 11, 12, 13, 14, 15, 16, 17, 18, 19, or 20.

The A² of an LC of Formula (II) may comprise a heterocyclyl group. The A² of an LC of Formula (II) may comprise a heteroaryl group. The heteroaryl or heterocyclyl group may comprise at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole.

The one or more lipids of an LC of Formula (II) may be selected from the group consisting of propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, myristic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid and hexatriacontanoic acid. The one or more lipids of an LC of Formula (II) may be selected from the group consisting of malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, pentadecanedioic acid, hexadecanedioic acid, heptadecanedioic acid, octadecanedioic acid, and nonadecanedioic acid. The one or more lipids of an LC of Formula (II) may be selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentanoic acid, erucic acid, docosahexaenoic acid. The one or more lipids of an LC of Formula (II) may be selected from the group consisting of cholesterol, 7-OH cholesterol, 7,25-dihydroxycholesterol, cholic acid, chenodeoxycholic acid, lithocholic acid, deoxycholic acid, glycocholic acid, glycodeoxycholic acid, glycolithocholic acid, and glycochenodeoxycholic acid. The one or more lipids of an LC of Formula (II) may be selected from the group consisting of cholic acid, chenodeoxycholic acid, lithocholic acid, deoxycholic acid, glycocholic acid, glycodeoxycholic acid, glycolithocholic acid, and glycochenodeoxycholic acid. The one or more lipids of an LC of Formula (II) may be selected from the group consisting of α-tocopherol, β-tocopherol, γ-tocopherol, δ-tocopherol, α-tocotrienol, β-tocotrienol, γ-tocotrienol and δ-tocotrienol. The one or more lipids of an LC of Formula (II) may be selected from the group consisting of octadecanedioic acid, tetradecylamine, myristic acid, docosahexaenoic acid, lithocholic acid ester, cholic acid and palmitic acid.

Disclosed herein are methods of producing an LC of Formula (II), the method comprising reacting an amino acid residue of TA with A³-P¹-L, wherein A³ is a reactive precursor to form A¹. In some embodiments, A³ is a haloacetamide, maleimide, benzyl halide, or pyridyl disulfide. In further embodiments, A³ is a haloacetamide. In still further embodiments, A³ is a bromoacetamide.

The lipid conjugates (LCs) may comprise (a) one or more lipids, the lipids selected from sterols, sterol derivatives, bile acids, vitamin E derivatives, fatty di-acids, fatty acids, fatty amides, and fatty alcohols; and (b) one or more therapeutic agents (TAs), wherein the one or more lipids are conjugated to the one or more therapeutic agents via an amino acid residue.

The lipid conjugates (LCs) may comprise (a) one or more lipids, the lipids selected from sterols, sterol derivatives, bile acids, vitamin E derivatives, fatty di-acids, fatty acids, fatty amides, and fatty alcohols; and (b) one or more therapeutic agents (TAs), wherein the one or more lipids are conjugated to the one or more therapeutic agents via a cysteine residue.

The lipid conjugates (LCs) may comprise (a) one or more lipids, the lipids selected from sterols, sterol derivatives, bile acids, vitamin E derivatives, fatty di-acids, fatty acids, fatty amides, fatty amines, and fatty alcohols, and derivatives thereof; (b) a therapeutic agent (TA); and (c) a linker connecting each of the one or more lipids to the TA, the linker comprising a heteroaryl or heterocyclyl group; wherein the TA is a peptide and each linker is conjugated to the TA via an amino acid residue on the peptide. The amino acid residue may be a cysteine, lysine, or serine. The amino acid residue may be selected from cysteine or lysine. The amino acid residue may be cysteine. The amino acid residue may be lysine. The amino acid may be an amino acid mutation. The amino acid may be an amino acid addition or an amino acid substitution. The amino acid may be located at the N-terminus or C-terminus of the peptide. The amino acid may be located at a non-terminus position of the peptide. The linker may further comprise one or more polyethyleneglycol subunits. The lipid may be pegylated. The linker may comprise a heterocyclyl group. The linker may comprise a heteroaryl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole ring.

The lipid conjugates (LC) may comprise (a) one or more lipids, the lipids selected from sterols, sterol derivatives, bile acids, vitamin E derivatives, fatty di-acids, fatty acids, fatty amides, and fatty alcohols; (b) one or more therapeutic agents (TAs); and (c) a linker connecting each of the one or more lipids to the TA, the linker comprising a heteroaryl or heterocyclyl group; wherein the one or more TAs comprise a relaxin peptide or derivative thereof. The lipid conjugates (LC) may comprise (a) one or more lipids, the lipids selected from sterols, sterol derivatives, bile acids, vitamin E derivatives, fatty di-acids, fatty acids, fatty amides, fatty amines, and fatty alcohols, and derivatives thereof (b) a therapeutic agent (TA); and (c) a linker connecting each of the one or more lipids to the TA, the linker comprising a heteroaryl or heterocyclyl group; wherein the TA comprises a relaxin peptide or derivative thereof. The amino acid may be located at the N-terminus or C-terminus of the peptide. The amino acid may be located at a non-terminus position of the peptide. The linker may further comprise one or more polyethyleneglycol subunits. The lipid may be pegylated. The linker may comprise a heterocyclyl group. The linker may comprise a heteroaryl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole ring.

The lipid conjugates (LC) may comprise (a) one or more lipids, the lipids selected from sterols, sterol derivatives, bile acids, vitamin E derivatives, fatty di-acids, fatty acids, fatty amides, and fatty alcohols; (b) one or more therapeutic agents (TAs); and (c) a linker connecting each of the one or more lipids to the TA, the linker comprising a heteroaryl or heterocyclyl group; wherein the one or more TAs comprise a relaxin peptide or derivative thereof, and each linker is attached to the one or more TAs via an amino acid residue. The lipid conjugates (LC) may comprise (a) one or more lipids, the lipids selected from sterols, sterol derivatives, bile acids, vitamin E derivatives, fatty di-acids, fatty acids, fatty amides, fatty amines, and fatty alcohols, and derivatives thereof (b) a therapeutic agent (TA); and (c) a linker connecting each of the one or more lipids to the TA, the linker comprising a heteroaryl or heterocyclyl group; wherein the TA comprises a relaxin peptide or derivative thereof, and each linker is attached to the TA via an amino acid residue on the peptide. The amino acid residue may be a cysteine, lysine, or serine. The amino acid residue may be selected from cysteine or lysine. The amino acid residue may be cysteine. The amino acid residue may be lysine. The amino acid may be an amino acid mutation. The amino acid may be an amino acid addition or an amino acid substitution. The amino acid may be located at the N-terminus or C-terminus of the peptide. The amino acid may be located at a non-terminus position of the peptide. The linker may further comprise one or more polyethyleneglycol subunits. The lipid may be pegylated. The linker may comprise a heterocyclyl group. The linker may comprise a heteroaryl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole ring.

The lipid conjugates (LC) may comprise one or more lipids attached to one or more therapeutic agents (TAs) and a linker connecting each of the one or more lipids to the TA, the linker comprising a heteroaryl or heterocyclyl group, wherein the one or more TAs comprise a modified relaxin peptide, and wherein the modified relaxin peptide comprises a wild-type relaxin polypeptide with one or more amino acid mutations. The lipid conjugates (LC) may comprise one or more lipids attached to a therapeutic agent (TA), wherein the TA comprises a modified relaxin peptide, and wherein the modified relaxin peptide comprises a wild-type relaxin polypeptide with one or more amino acid mutations.

The LCs disclosed herein may comprise a TA comprising a modified relaxin peptide. The LCs disclosed herein may comprise one or more lipids, wherein the one or more lipids may be selected from the group consisting of sterols, sterol derivatives, bile acids, vitamin E derivatives, fatty acids and fatty alcohols, and derivatives thereof. The attachment of the one or more lipids to the modified relaxin peptide may comprise covalent attachment. The one or more lipids may be attached to the modified relaxin peptide via a cysteine residue. The cysteine residue may be an amino acid mutation. The cysteine residue may be an amino acid addition or an amino acid substitution. The cysteine residue may be an amino acid addition or substitution on a wild-type peptide. The amino acid may be located at the N-terminus or C-terminus of the peptide. The amino acid may be located at a non-terminus position of the peptide. The linker may further comprise one or more polyethyleneglycol subunits. The lipid may be pegylated. The linker may comprise a heterocyclyl group. The linker may comprise a heteroaryl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole ring.

The LCs disclosed herein may have the structure:

TA-A¹-P¹-L Formula (III)

wherein TA is the therapeutic agent comprising at least one isosteric disulfide mimetic; A¹ is a chemical group linking TA and P¹ or L; P¹ is a bond or comprises polyglycol; and L is the lipid. P¹ may be a bond. A sulfur or nitrogen atom of an amino acid residue of TA may be connected to A¹ via a chemical bond. A¹ may be selected from

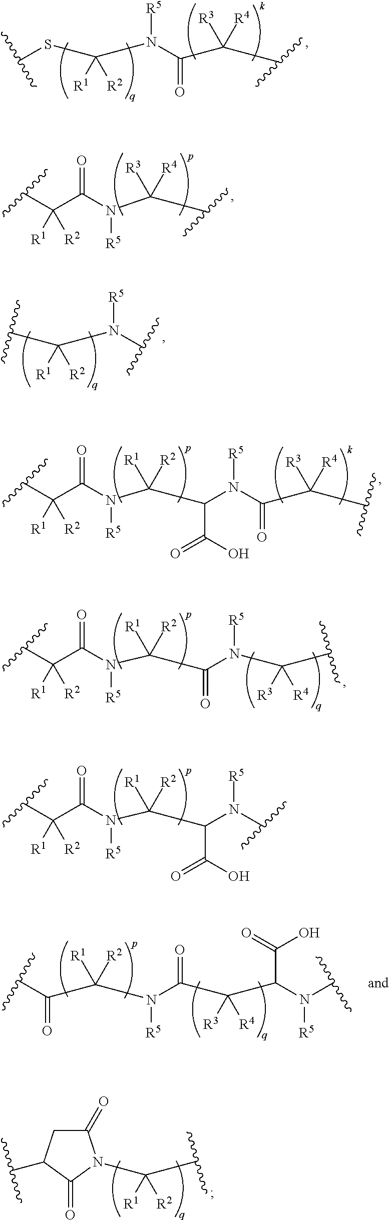

wherein each R¹, R², R³, and R⁴ is independently selected from H, halo, CN, —SR⁵, alkyl, cycloalkyl, haloalkyl, —NR⁵R⁵, —NC(O)R⁵, —NC(O)OR⁵, and —OR⁵; each R⁵ is independently H, alkyl, haloalkyl, arylalkyl, (cycloalkyl)alkyl, or heteroalkyl; k is 1, 2, 3, 4, 5, 6, 7, 8, 9, or 10; p is 2, 3, 4, 5, 6, 7, 8, 9, or 10; and q is 2, 3, 4, 5, 6, 7, 8, 9, or 10. P¹ may comprise polyglycol. P¹ may be -PEG-A²-; PEG may be a chemical group comprising one or more polyethyleneglycol subunits; and A² may be a bond or a chemical group linking PEG and L. PEG may be selected from

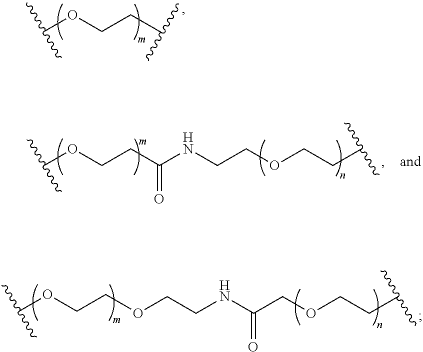

wherein m and n are independently 1, 2, 3, 4, 5, 6, 7, 8, 9, 10, 11, 12, 13, 14, 15, 16, 17, 18, 19, or 20. A² may be selected from a bond,

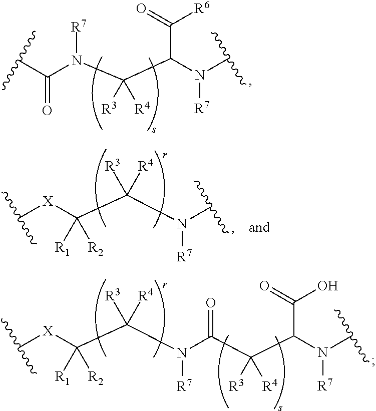

wherein X is a bond, NR⁵, S, or O; each R¹, R², R³, and R⁴ is independently selected from H, halo, CN, —SR⁵, alkyl, cycloalkyl, haloalkyl, —NR⁵R⁵, and —OR⁵; each R⁵ is independently H, alkyl, haloalkyl, arylalkyl, or heteroalkyl; R⁶ is OH or —NR⁵R⁵; each R⁷ is independently selected from H, alkyl, haloalkyl, arylalkyl, and heteroalkyl; r is 1, 2, 3, 4, 5, 6, 7, 8, 9, or 10; and s is 1, 2, 3, 4, or 5. The lipid may be selected from the group consisting of octadecanedioic acid, tetradecylamine, myristic acid, stearic acid, docosahexaenoic acid, lithocholic acid ester, cholic acid and palmitic acid. The isosteric disulfide mimetic may comprise a heteroaryl group. The heteroaryl group may comprise at least one nitrogen. The heteroaryl group may be a 5-membered ring comprising at least one nitrogen.

### Linker

The LCs disclosed herein may further comprise one or more linkers. The LCs disclosed herein may further comprise two or more linkers. The LCs disclosed herein may further comprise three or more linkers. The LCs disclosed herein may further comprise four or more linkers. LCs disclosed herein may further comprise five or more linkers.

The one or more linkers may enable attachment of a lipid to a therapeutic agent. The linker may enable attachment of a lipid to another lipid. The linker may enable attachment of a lipid to a chemical group comprising one or more polyethyleneglycol subunits. The linker may enable attachment of a PEG to another PEG. The linker may enable attachment of a PEG to a therapeutic agent. The linker may enable attachment of a therapeutic agent to another therapeutic agent. The linker may be an amino acid. The linker may be an amino acid of the therapeutic agent. The linker may be an amino acid mutation of the therapeutic agent. The linker may be a substituted amino acid of the therapeutic agent. The linker may be an amino acid addition of the therapeutic agent. The linker may be an amino acid mutation located at the C-terminus of the peptide. The linker may be an amino acid mutation located at the N-terminus of the peptide. The linker may be an amino acid mutation located at a non-terminus position of the peptide. The linker may be a lysine. The linker may be a cysteine. The linker may be an L-cysteine. The linker may be an ether or an amide. The linker may link a PEG molecule to a lipid.

The linker may comprise a heteroaryl or heterocyclyl group. The linker may comprise a heterocyclyl group. The linker may comprise a heteroaryl group. The heteroaryl or heterocyclyl group may comprise at least one nitrogen. The heteroaryl or heterocyclyl group may be a 5-membered ring comprising at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole ring.

### Lipid Derivatives

The LCs disclosed herein may comprise one or more lipid derivatives. The lipid derivatives may be attached to a TA. Attachment of the lipid derivative to the TAs may enhance the pharmacokinetic properties of the TAs. Lipid derivatives may comprise polyglycol. Lipid derivatives may be pegylated. A pegylated lipid may comprise at least one polyethyleneglycol subunit. The lipid derivatives may be not pegylated. Lipids may be broadly defined as hydrophobic or amphiphilic small molecules. Lipids may be naturally occurring or synthetic. Lipids may be eicosanoids, prostaglandins, leukotrienes, thromboxanes, wax esters, coenzyme A derivatives, fatty acid carnitines, fatty acid amides, ethanolamines, bile acids, vitamin E, vitamin A, vitamin D, vitamin K, fat-soluble vitamin derivatives, monoglycerides, diglycerides, triglycerides, phospholipids, phosphatidylcholine, glycerolipids, glycerols, glycerophospholypids, sphingolipids, saccharolipids, polyketides, sterols, sterol derivatives, sterol lipids, steroid hormones, prenol lipids, carotenoids, fatty acids, and fatty alcohols.

In one aspect, disclosed herein are lipid derivatives having the structure of A³-P¹-L, wherein:
A³ is a haloacetamide, maleimide, benzyl halide, or pyridyl disulfide;
P¹ comprises a heteroaryl or heterocyclyl group; and
L is a lipid selected from sterols, sterol derivatives, bile acids, vitamin E derivatives, fatty di-acids, fatty acids, fatty amides, and fatty alcohols, and derivatives thereof.

In some embodiments, P¹ is -PEG-A²-, wherein PEG is a chemical group comprising one or more polyethyleneglycol subunits and A² is a chemical group linking PEG and L. In some embodiments described herein, PEG is selected from:

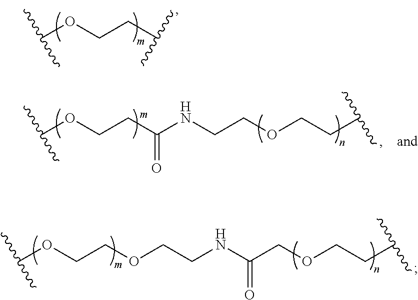

wherein
m and n are independently 1, 2, 3, 4, 5, 6, 7, 8, 9, 10, 11, 12, 13, 14, 15, 16, 17, 18, 19, or 20.

A² may comprise a heteroaryl or heterocyclyl group. A² may comprise a heterocyclyl group. A² may comprise a heteroaryl group. The heteroaryl or heterocyclyl group may comprise at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole.

In some embodiments described herein,
A² is selected from a bond,

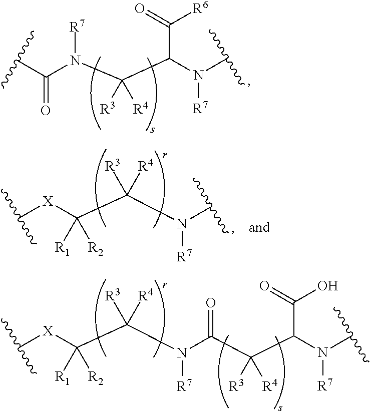

X is a bond, NR⁵, S, or O;
each R¹, R², R³, and R⁴ is independently selected from H, halo, CN, —SR⁵, alkyl, cycloalkyl, haloalkyl, —NR⁵R⁵, and —OR⁵;
each R⁵ is independently H, alkyl, haloalkyl, arylalkyl, or heteroalkyl;
R⁶ is OH or —NR⁵R⁵;
each R⁷ is independently selected from H, alkyl, haloalkyl, arylalkyl, and heteroalkyl;
r is 1, 2, 3, 4, 5, 6, 7, 8, 9, or 10; and
s is 1, 2, 3, 4, or 5.

### Lipids

The LCs disclosed herein may comprise one or more lipids. The one or more lipids may be fatty acids. The fatty acids (FAs) may be attached to one or more therapeutic agents (TAs). Attachment of the fatty acids to the TAs may enhance the pharmacokinetic properties of the TAs. Fatty acids may be fatty di-acids, fatty amides, fatty amines, or fatty alcohols. Fatty acids may be saturated or unsaturated. Saturated fatty acids include, but are not limited to, lauric acid, myristic acid, palmitic acid, stearic acid, arachidic acid. Unsaturated fatty acids include, but are not limited to palmitoleic acid, oleic acid, linoleic acid, linolenic acid, erucic acid and arachidonic acid. Fatty acids may be short-chain fatty acids, medium chain fatty acids, long chain fatty acids or very long chain fatty acids. Fatty acids may be monounsaturated or polyunsaturated. Fatty acids may be omega fatty acids, essential fatty acids, partially hydrogenated fatty acids, cis-isomer fatty acids, or trans-isomer fatty acids. Fatty acids may be omega-3 fatty acids, omega-6 fatty acids or omega-9 fatty acids. Fatty acids may be dicarboxylic acids.

The fatty acid may comprise a chain of about 6, 7, 8, 9, 10, 11, 12, 13, 14, 15, 16, 17, 18, 19, 20, 21, 22, 23, 24, 25, 26 or more carbon atoms. The fatty acid may comprise a carbon chain further comprising 1, 2, 3, 4, 5, 6 or more double bonds. The fatty acid may be naturally occurring. The fatty acid may not be naturally occurring. The fatty acid may be synthesized.

The LCs disclosed herein may further comprise one or more fatty acids. The LCs disclosed herein may further comprise two or more fatty acids. The LCs disclosed herein may further comprise three or more fatty acids. The LCs disclosed herein may further comprise four or more fatty acids. LCs disclosed herein may further comprise five or more fatty acids. The fatty acids may be different. The fatty acids may be the same.

The one or more lipids of any LC may be selected from the group consisting of myristic acid, docosahexanoic acid, lithocholic acid ester, cholic acid and palmitic acid. The one or more lipids of any LC may be myristic acid. The one or more lipids of any LC may be docosahexanoic acid. The one or more lipids of any LC may be lithocholic acid ester. The one or more lipids of any LC may be cholic acid. The one or more lipids of any LC may be palmitic acid.

The LCs may comprise one or more sterols or sterol derivatives. The sterols or sterol derivatives may be selected from the group consisting of cholesterol, 7-OH cholesterol, 7,25-dihydroxycholesterol, cholic acid, chenodeoxycholic acid, lithocholic acid, deoxycholic acid, glycocholic acid, glycodeoxycholic acid, glycolithocholic acid, and glycochenodeoxycholic acid.

The LCs may comprise one or more bile acids. The bile acids may be selected from the group consisting of cholic acid, chenodeoxycholic acid, lithocholic acid, deoxycholic acid, glycocholic acid, glycodeoxycholic acid, glycolithocholic acid, and glycochenodeoxycholic acid.

The LC may comprise one or more Vitamin E derivatives. The Vitamin E derivatives may be selected from the group consisting of α-tocopherol, β-tocopherol, γ-tocopherol, δ-tocopherol, α-tocotrienol, β-tocotrienol, γ-tocotrienol and δ-tocotrienol.

### Pegylated Lipid

The LCs disclosed herein in may comprise one or more pegylated lipids. A pegylated lipid may comprise at least one polyethyleneglycol subunit. The connection between the lipid and the one or more polyethyleneglycol subunits may be a direct bond or a linker (A²). Non-limiting examples of a linker between the lipid and the one or more polyethyleneglycol subunits include: a bond,

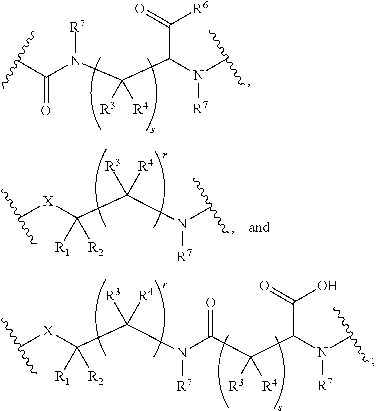

wherein
X is a bond, NR⁵, S, or O;
each R¹, R², R³, and R⁴ is independently selected from H, halo, CN, —SR⁵, alkyl, cycloalkyl, haloalkyl, —NR⁵R⁵, and —OR⁵;
each R⁵ is independently H, alkyl, haloalkyl, arylalkyl, or heteroalkyl;
R⁶ is OH or —NR⁵R⁵;
each R⁷ is independently selected from H, alkyl, haloalkyl, arylalkyl, and heteroalkyl;
r is 1, 2, 3, 4, 5, 6, 7, 8, 9, or 10; and
s is 1, 2, 3, 4, or 5.

A pegylated lipid may have the structure P¹-L, wherein P¹ is -PEG-A²-; PEG is a chemical group comprising one or more polyethyleneglycol subunits; A² is a chemical group linking PEG and L; and L is a lipid. PEG may be selected from:

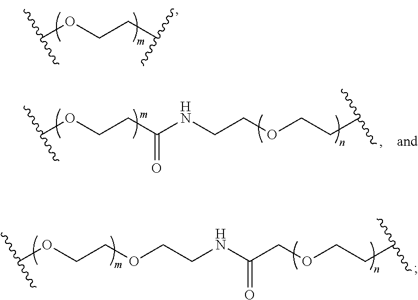

wherein
m and n are independently 1, 2, 3, 4, 5, 6, 7, 8, 9, 10, 11, 12, 13, 14, 15, 16, 17, 18, 19, or 20. A² may be a heteroaryl or heterocyclyl group. The heteroaryl or heterocyclyl group may comprise at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole.

A pegylated lipid may be connected to a therapeutic agent through a linker. The linker may comprise one or more amide moieties. One or more amide moieties may be replaced by a heteroaryl or heterocyclyl group. The heteroaryl or heterocyclyl group may comprise at least one nitrogen. The heteroaryl or heterocyclyl group may be selected from a pyrrole, pyrazole, imidazole, triazole, thiazole, oxazole, and isoxazole. The heteroaryl or heterocyclyl group may be a triazole.

### Therapeutic Agent (TA)

The modified therapeutic agents disclosed herein may comprise one or more therapeutic agents. The modified therapeutic agents may comprise two or more therapeutic agents. The modified therapeutic agents may comprise 3, 4, 5, 6, 7 or more therapeutic agents. The therapeutic agents may be different. The therapeutic agents may be the same. As used herein, the term “therapeutic agent” refers to candidate proteins or peptides that modulate the activity of a target protein, target peptide, target cell or target tissue. Modulating the activity can comprise increasing, decreasing, stimulating, or preventing the activity or expression of the target protein, peptide, cell or tissue. Target proteins or peptides include proteins involved in the etiology of a disease or disorder by virtue of expression or activity. TAs may comprise at least a portion of a protein, biomolecule, chemical, toxin, drug or any combination thereof. Exemplary TAs may include, but are not limited to, at least a portion of a hormone, kinase, receptor, ligand, growth factor, regulatory protein, metabolic protein, cytokine, chemokine, interferon, phosphatase, antibody, peptidyl toxin, or any combination thereof. The TA may be a wild-type peptide or a modified peptide comprising one or more amino acid additions, deletions, substitutions, or a combination thereof. The one or more amino acid additions or substitutions may be located at the C-terminus of the peptide. The one or more amino acid additions or substitutions may be located at the N-terminus of the peptide. The one or more amino acid additions or substitutions may be located at the N-terminus or the C-terminus of the peptide. The one or more amino acid additions or substitutions may be located at both the N-terminus and C-terminus of the peptide. The one or more amino acid additions or substitutions may be located at a non-terminus position of the peptide.

The TA may be a hormone. Examples of hormones include, but are not limited to, peptide hormones, lipid and phospholipid-derived hormones, and monoamines. Peptide hormones generally consist of chains of amino acids. Examples of small peptide hormones include, but are not limited to thyrotropin-releasing hormone (TRH) and vasopressin. Peptides composed of scores or hundreds of amino acids may be referred to as proteins. Examples of protein hormones include insulin and growth hormone. More complex protein hormones may bear carbohydrate side-chains and may be called glycoprotein hormones. Luteinizing hormone, follicle-stimulating hormone and thyroid-stimulating hormone are examples of glycoprotein hormones. Lipid and phospholipid-derived hormones are generally derived from lipids such as linoleic acid and arachidonic acid and phospholipids. Protein hormones may comprise steroid hormones that are derived from cholesterol and the eicosanoids. Examples of steroid hormones are testosterone and cortisol. Eicosanoids may comprise prostaglandins. Monoamines may be derived from aromatic amino acids like phenylalanine, tyrosine, tryptophan by the action of aromatic amino acid decarboxylase enzymes. The TA may be oxyntomodulin. The TA may be exendin-4. The TA may be exenatide. The TA may be glucagon-like peptide (GLP-1). The TA may be glucagon. The TA may be leptin. The TA may be betatrophin.

The TA may be a relaxin peptide or derivative thereof. The relaxin peptide may comprise a modified relaxin peptide. The modified relaxin peptide may comprise at least a portion of a wild-type relaxin peptide comprising one or more amino acid mutations. The relaxin peptide may comprise at least a portion of an A chain and/or B chain of a relaxin peptide. The relaxin peptide may comprise one or more amino acid mutations. The one or more amino acid mutations may comprise a deletion, substitution, addition or a combination thereof. The one or more amino acid mutations may comprise addition of one or more amino acid residues to the wild-type relaxin polypeptide. The one or more amino acid mutations may comprise substitution of one or more amino acid residues of the wild-type relaxin polypeptide. The one or more amino acid mutations may comprise deletion of one or more amino acid residues of the wild-type relaxin polypeptide. The one or more amino acid mutations may comprise one or more amino acid substitutions of one or more amino acid residues in an A chain and/or B chain of a wild-type relaxin peptide. The one or more amino acid mutations may comprise one or more amino acid substitutions of one or more amino acid residues in an A chain of a wild-type relaxin peptide. The one or more amino acid substitutions of one or more amino acid residues in the A chain may be selected from the group consisting of Y3C, A7C, T16C, R18C, S19C, or a combination thereof. The one or more amino acid mutations may comprise one or more amino acid substitutions of one or more amino acid residues in a B chain of a wild-type relaxin peptide. The one or more amino acid substitutions of one or more amino acid residues in the B chain may be selected from the group consisting of D1A, S2C, M4C, S26C, and S29C, or any combination thereof. The one or more amino acid mutations may comprise a Y3C substitution in an A chain of a wild-type relaxin peptide. The one or more amino acid mutations may comprise an A7C substitution in an A chain of a wild-type relaxin peptide. The one or more amino acid mutations may comprise a T16C substitution in an A chain of a wild-type relaxin peptide. The one or more amino acid mutations may comprise a R18C substitution in an A chain of a wild-type relaxin peptide. The one or more amino acid mutations may comprise a S19C substitution in an A chain of a wild-type relaxin peptide. The one or more amino acid mutations may comprise a S2C substitution in a B chain of a wild-type relaxin peptide. The one or more amino acid mutations may comprise a D1A substitution in a B chain of a wild-type relaxin peptide. The one or more amino acid mutations may comprise a M4C substitution in a B chain of a wild-type relaxin peptide. The one or more amino acid mutations may comprise a S26C substitution in a B chain of a wild-type relaxin peptide. The one or more amino acid mutations may comprise a S29C substitution in a B chain of a wild-type relaxin peptide. The one or more amino acid mutations may comprise substituting one or more amino acid residues of a wild-type relaxin peptide with a cysteine residue. The one or more amino acid residues of the wild-type relaxin peptide are selected from the group consisting of alanine, methionine, arginine, serine, threonine, and tyrosine. The one or more amino acid mutations may comprise adding one or more amino acid residues to a wild-type relaxin peptide.

The TA may be a growth factor. Growth factors may include, but are not limited to, cytokines and hormones. Examples of growth factors include, but are not limited to, adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), migration-stimulating factor, myostatin (GDF-8), nerve growth factor (NGF) and other neurotrophins, platelet-derived growth factor (PDGF), thrombopoietin (TPO), transforming growth factor alpha (TGF-α), transforming growth factor beta (TGF-β), tumor necrosis factor-alpha (TNF-α) and vascular endothelial growth factor (VEGF). The TA may be fibroblast growth factor 21 (FGF21).

The TA may be a cell regulatory protein. The TA may be a cell regulatory protein of the transforming growth factor beta superfamily. The TA may be a member of the decapentaplegic-Vg related (DVR) related subfamily. The TA may be a member of the activin/inhibin subfamily. The TA may be a member of the TGF-beta subfamily. The TA may be a growth differentiation factor (GDF). The GDF may be GDF1, GDF2, GDF3, GDF5, GDF6, GFD8, GDF9, GDF10, GDF11, and GDF15. The TA may be growth differentiation factor 11 (GDF11).

The TA may be a peptidyl toxin. Examples include, but not limited to, Toxin-550, Moka, and VM-24.

The TA may be a protein. The protein may be a member of the angiopoietin-like family of secreted factors. The protein may be an angiopoietin-like protein (ANGPTL). Examples of ANGPTLs include, but are not limited to, ANGPTL1, ANGPTL2, ANGPTL3, ANGPTL4, ANGPTL5, ANGPTL6 and ANGPTL7. The TA may be ANGPTL3.

The TA may comprise a peptide selected from relaxin, H1 relaxin, H2 relaxin, H3 relaxin, human INSL3, human INSL4, human INSL6, human IGF1, human IGFII, human insulin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, peptide-based toxin, Moka, and VM-24, and derivatives thereof, the derivative being a peptide comprising one or more amino acid additions, deletions, or substitutions, or a combination thereof. The TA may comprise relaxin, oxyntomodulin, exenatide, exendin-4, glucagon-like protein-1 (GLP-1), GLP-2, glucagon, a GLP-1R and GIPR dual agonist, a GLP-1R and GCGR dual agonist, leptin, betatrophin, FGF 21, GDF 11, ANGPTL3, peptide-based toxin, Moka, or VM-24, or a derivative thereof, the derivative being a peptide comprising one or more amino acid additions, deletions, or substitutions, or a combination thereof. The TA may comprise oxyntomodulin, exenatide, exendin-4, glucagon-like protein-1 (GLP-1), GLP-2, glucagon, a GLP-1R and GIPR dual agonist, or a GLP-1R and GCGR dual agonist, or a derivative thereof, the derivative being a peptide comprising one or more amino acid additions, deletions, or substitutions, or a combination thereof. The TA may comprise H1 relaxin, H2 relaxin, H3 relaxin, human INSL3, human INSL4, human INSL6, human IGF1, human IGFII, or human insulin, or a derivative thereof, the derivative being a peptide comprising one or more amino acid additions, deletions, or substitutions, or a combination thereof. The TA may comprise H1 relaxin, H2 relaxin, or H3 relaxin, or a derivative thereof, the derivative being a peptide comprising one or more amino acid additions, deletions, or substitutions, or a combination thereof. The TA may comprise human INSL3, human INSL4, human INSL6, human IGF1, or human IGFII, or a derivative thereof, the derivative being a peptide comprising one or more amino acid additions, deletions, or substitutions, or a combination thereof. The TA may comprise a peptidyl toxin or a derivative thereof, the derivative being a peptide comprising one or more amino acid additions, deletions, or substitutions, or a combination thereof. The TA may comprise Toxin-550, Moka, or VM-24, or a derivative thereof, the derivative being a peptide comprising one or more amino acid additions, deletions, or substitutions, or a combination thereof. The TA may comprise human insulin or a derivative thereof, the derivative being a peptide comprising one or more amino acid additions, deletions, or substitutions, or a combination thereof.

The TA may be encoded by a nucleotide sequence based on or derived from a nucleotide sequence selected from the group comprising SEQ ID NO: 1-9. The TA may be encoded by a nucleotide sequence that is at least 50% homologous to a nucleotide sequence selected from the group comprising SEQ ID NO: 1-9. The TA may be encoded by a nucleotide sequence that is at least 55%, 60%, 65%, 70%, 75%, 80%, 85%, 90%, 92%, 95%, 97%, 99%, or 100% homologous to a nucleotide sequence selected from the group comprising SEQ ID NO: 1-9. The TA may be encoded by a nucleotide sequence that is at least 70% homologous to a nucleotide sequence selected from the group comprising SEQ ID NO: 1-9. The TA may be encoded by a nucleotide sequence that is at least 75% homologous to a nucleotide sequence selected from the group comprising SEQ ID NO: 1-9. The TA may be encoded by a nucleotide sequence that is at least 80% homologous to a nucleotide sequence selected from the group comprising SEQ ID NO: 1-9. The TA may comprise one or more nucleotide sequences selected from the group comprising SEQ ID NO: 1-9. The TA may comprise two or more nucleotide sequences selected from the group comprising SEQ ID NO: 1-9. The TA may comprise three or more nucleotide sequences selected from the group comprising SEQ ID NO: 1-9. The TA may comprise four or more nucleotide sequences selected from the group comprising SEQ ID NO: 1-9. The TA may comprise 5, 6, 7, 8, or 9 nucleotide sequences selected from the group comprising SEQ ID NO: 1-9.

The TA may comprise an amino acid sequence selected from the group comprising SEQ ID NO: 10-61. The TA may comprise an amino acid sequence that is at least 50% homologous to an amino acid sequence selected from the group comprising SEQ ID NO: 10-61. The TA may comprise an amino acid sequence that is at least 55%, 60%, 65%, 70%, 75%, 80%, 85%, 90%, 92%, 95%, 97%, 99%, or 100% homologous to an amino acid sequence selected from the group comprising SEQ ID NO: 10-61. The TA may comprise an amino acid sequence that is at least 70% homologous to an amino acid sequence selected from the group comprising SEQ ID NO: 10-61. The TA may comprise an amino acid sequence that is at least 75% homologous to an amino acid sequence selected from the group comprising SEQ ID NO: 10-61. The TA may comprise an amino acid sequence that is at least 80% homologous to an amino acid sequence selected from the group comprising SEQ ID NO: 10-61. The TA may comprise one or more amino acid sequences selected from the group comprising SEQ ID NO: 10-61. The TA may comprise two or more amino acid sequences selected from the group comprising SEQ ID NO: 10-61. The TA may comprise three or more amino acid sequences selected from the group comprising SEQ ID NO: 10-61. The TA may comprise four or more amino acid sequences selected from the group comprising SEQ ID NO: 10-61. The TA may comprise 5, 6, 7, 8, 9, 10, 11, 12, 13, 14, or 15 or more amino acid sequences selected from the group comprising SEQ ID NO: 10-61.

The TA may comprise 20 or more consecutive amino acids from an amino acid sequence selected from the group comprising SEQ ID NO: 10-61. The TA may comprise 30, 40, 50, 60, 70, 80, 90, 100, 110, 120, 130 or more consecutive amino acids from an amino acid sequence selected from the group comprising SEQ ID NO: 10-61.

The TA may comprise an amino acid sequence selected from the group consisting of SEQ ID NO: 16, 40, 48-52, 54, 56, 55, 58, and 59-61. The TA may comprise an amino acid sequence that is at least 50% homologous to an amino acid sequence selected from the group consisting of SEQ ID NO: 16, 40, 48-52, 54, 56, 55, 58, and 59-61. The TA may comprise an amino acid sequence that is at least 55%, 60%, 65%, 70%, 75%, 80%, 85%, 90%, 92%, 95%, 97%, 99%, or 100% homologous to an amino acid sequence selected from the group consisting of SEQ ID NO: 16, 40, 48-52, 54, 56, 55, 58, and 59-61. The TA may comprise an amino acid sequence that is at least 70% homologous to an amino acid sequence selected from the group consisting of SEQ ID NO: 16, 40, 48-52, 54, 56, 55, 58, and 59-61. The TA may comprise an amino acid sequence that is at least 75% homologous to an amino acid sequence selected from the group consisting of SEQ ID NO: 16, 40, 48-52, 54, 56, 55, 58, and 59-61. The TA may comprise an amino acid sequence that is at least 80% homologous to an amino acid sequence selected from the group consisting of SEQ ID NO: 16, 40, 48-52, 54, 56, 55, 58, and 59-61. The TA may comprise one or more amino acid sequences selected from the group consisting of SEQ ID NO: 16, 40, 48-52, 54, 56, 55, 58, and 59-61. The TA may comprise two or more amino acid sequences selected from the group consisting of SEQ ID NO: 16, 40, 48-52, 54, 56, 55, 58, and 59-61. The TA may comprise two or more amino acid sequences selected from the group consisting of SEQ ID NO: 16, 40, 48-52, 54, 56, 55, 58, and 59-61. The TA may comprise three or more amino acid sequences selected from the group consisting of SEQ ID NO: 16, 40, 48-52, 54, 56, 55, 58, and 59-61. The TA may comprise 20 or more consecutive amino acids from an amino acid sequence selected from the group consisting of SEQ ID NO: 16, 40, 48-52, 54, 56, 55, 58, and 59-61. The TA may comprise 19, 18, 17, 16, 15, 14, 13, 12, 11, 10, 9, 8, 7, 6, or 5 or more consecutive amino acids from an amino acid sequence selected from the group comprising SEQ ID NO: 16, 40, 48-52, 54, 56, 55, 58, and 59-61.

The TA may comprise the amino acid sequence SEQ ID NO: 54. The TA may comprise the amino acid sequence SEQ ID NO: 54 and the amino acid sequence SEQ ID NO: 48. The TA may comprise the amino acid sequence SEQ ID NO: 55. The TA may comprise the amino acid sequence SEQ ID NO: 55 and the amino acid sequence SEQ ID NO: 48. The TA may comprise the amino acid sequence SEQ ID NO: 59. The TA may comprise the amino acid sequence SEQ ID NO: 59 and the amino acid sequence SEQ ID NO: 48.

The TA may comprise the amino acid sequence SEQ ID NO: 61. The TA may comprise the amino acid sequence SEQ ID NO: 61 and one or more amino acid sequences selected from the group consisting of SEQ ID NO: 10-60. The TA may comprise the amino acid sequence SEQ ID NO: 61 and one amino acid sequence selected from the group consisting of SEQ ID NO: 10-60. The TA may comprise the amino acid sequence SEQ ID NO: 61 and the amino acid sequence SEQ ID NO: 45. The TA may comprise the amino acid sequence SEQ ID NO: 61 and the amino acid sequence SEQ ID NO: 54. The TA may comprise the amino acid sequence SEQ ID NO: 61 and the amino acid sequence SEQ ID NO: 55. The TA may comprise the amino acid sequence SEQ ID NO: 61 and the amino acid sequence SEQ ID NO: 59.

The TAs may be from a mammal or non-mammal. The TAs may be from a human. Alternatively, the TAs may be from a goat, sheep, cow, rabbit, monkey, dog, cat or a combination thereof. The TAs may be from a reptile. The TAs may be from a snake or lizard. The TAs may be from an amphibian. The TAs may be from a frog or toad. The TAs may be from an avian. The TAs may be recombinant.

The TAs disclosed herein may further comprise one or more linkers. The TAs disclosed herein may further comprise two or more linkers. The TAs disclosed herein may further comprise three or more linkers. The TAs disclosed herein may further comprise four or more linkers. The TAs disclosed herein may further comprise five or more linkers.

Therapeutic Agent (TA) with Disulfide Mimetic

The TA may further comprise one or more isosteric disulfide mimetics. The isosteric disulfide mimetic may comprise a heteroaryl group.

### Pharmacokinetics

Mechanisms by which the modified therapeutic agents positively influence pharmacokinetic or pharmacodynamic behavior include, but are not limited to, (i) preventing or mitigating in vivo proteolytic degradation or other activity-diminishing chemical modification of the therapeutic agent; (ii) improving half-life or other pharmacokinetic properties by reducing renal filtration, decreasing receptor-mediated clearance or increasing bioavailability; (iii) reducing toxicity; (iv) improving solubility; and/or (v) increasing biological activity and/or target selectivity of the therapeutic agent.

The half-life extending moieties may enhance one or more pharmacokinetic properties of a therapeutic agent (TA) when attached to the TA.

The modified therapeutic agents disclosed herein may enhance the one or more pharmacokinetic properties of the TA by at least about 200% as measured by pharmacodynamics when compared to the TA alone. The modified therapeutic agents disclosed herein may enhance the one or more pharmacokinetic properties of the TA by at least about 300%, 400%, 500%, 600%, 700%, 800%, 900%, 1000% as measured by pharmacodynamics when compared to the TA alone. The modified therapeutic agents disclosed herein may enhance the one or more pharmacokinetic properties of the TA by at least about 250% as measured by pharmacodynamics when compared to the TA alone. The modified therapeutic agents disclosed herein may enhance the one or more pharmacokinetic properties of the TA by at least about 300% as measured by pharmacodynamics when compared to the TA alone. The modified therapeutic agents disclosed herein may enhance the one or more pharmacokinetic properties of the TA by at least about 350% as measured by pharmacodynamics when compared to the TA alone. The modified therapeutic agents disclosed herein may enhance the one or more pharmacokinetic properties of the TA by at least about 400% as measured by pharmacodynamics when compared to the TA alone. The modified therapeutic agents disclosed herein may enhance the one or more pharmacokinetic properties of the TA by at least about 500% as measured by pharmacodynamics when compared to the TA alone.

The pharmacokinetic properties may comprise a half-life. The half-life of the modified therapeutic agent may be at least about two-fold longer compared to the half-life of the TA alone. The half-life of the modified therapeutic agent disclosed herein may be at least about 3-fold, 4-fold, or 5-fold longer compared to the half-life of the TA alone. The half-life of the modified therapeutic agent disclosed herein may be at least about 6-, 7-, 8-, 9-, 10-, 15-, 20-, 25-, 30-, 35-, 40-, 45-, or 50-fold longer compared to the half-life of the TA alone. The half-life of the modified therapeutic agent disclosed herein may be at least about 5-fold longer compared to the half-life of the TA alone. The half-life of the modified therapeutic agent disclosed herein may be at least about 10-fold longer compared to the half-life of the TA alone.

In addition, the modified therapeutic agents may have positive effects on terms of increasing manufacturability, and/or reducing immunogenicity of the therapeutic agent compared to an unconjugated form of the therapeutic agent.

A modified therapeutic agent may have comparable activity to the TA alone. A modified therapeutic agent may have similar activity to the TA alone. A modified therapeutic agent may have increased activity to the TA alone. A modified therapeutic agent may have decreased activity to the TA alone.

Attachment of a half-life extending moiety to a TA to form an a modified therapeutic agent may diminish the activity of the modified therapeutic agent relative to the TA alone by no more than 2-, 3-, 4-, 5-, 6-, 7-, 8-, 9-, or 10-fold less activity. Attachment of one or more lipids to a TA to form an LC may diminish the activity of the LC relative to the TA alone by no more than 2-, 3-, 4-, 5-, 6-, 7-, 8-, 9-, or 10-fold less activity. Attachment of a polyglycol region to a TA to form a modified therapeutic agent may diminish the activity of the modified therapeutic agent relative to the TA alone by no more than 2-, 3-, 4-, 5-, 6-, 7-, 8-, 9-, or 10-fold less activity.

### Therapeutic Use

Further disclosed herein are modified therapeutic agents for treating, alleviating, inhibiting and/or preventing one or more diseases and/or conditions. The disease and/or condition may be a chronic disease or condition. Alternatively, the disease and/or condition may be an acute disease or condition. The disease or condition may be recurrent, refractory, accelerated, or in remission. The disease or condition may affect one or more cell types. The one or more diseases and/or conditions may be an autoimmune disease, inflammatory disease, cardiovascular disease and pregnancy. The modified therapeutic agents disclosed herein may be administered to a subject in need thereof.

Disclosed herein are methods of treating a disease or condition in a subject in need thereof, the method comprising administering to the subject a composition comprising an LC, wherein the LC comprises a lipid attached to a therapeutic agent, wherein the therapeutic agent is relaxin or a derivative thereof.

Disclosed herein are methods of treating a disease or condition in a subject in need thereof, the method comprising administering to the subject a composition comprising an modified therapeutic agent, wherein the modified therapeutic agent comprises (i) a therapeutic agent (TA) comprising an amino acid sequence selected from the group consisting of SEQ ID NO: 54, 55, 59 and 60; and a (ii) half-life extending moiety.

Disclosed herein are methods of treating a disease or condition in a subject in need thereof, the method comprising administering to the subject a composition comprising an modified therapeutic agent, wherein the modified therapeutic agent comprises (i) a therapeutic agent (TA) comprising an amino acid sequence that is at least 50% homologous to an amino acid sequence selected from the group consisting of SEQ ID NO: 54, 55, 59 and 60; and a (ii) half-life extending moiety.

Disclosed herein are methods of treating a disease or condition in a subject in need thereof, the method comprising administering to the subject a composition comprising an modified therapeutic agent, wherein the modified therapeutic agent comprises (i) a therapeutic agent (TA) comprising an amino acid sequence selected from the group consisting of SEQ ID NO: 54, 55, 59 and 60 wherein up to five amino acids have been added, deleted, substituted, or a combination thereof; and a (ii) half-life extending moiety.

Disclosed herein are methods of treating a disease or condition in a subject in need thereof, the method comprising administering to the subject a composition comprising an modified therapeutic agent, wherein the modified therapeutic agent comprises (i) a therapeutic agent (TA) comprising a portion of an amino acid sequence selected from the group consisting of SEQ ID NO: 54, 55, 59 and 60; and a (ii) half-life extending moiety.

The disease or condition may be a cardiovascular disease. The cardiovascular disease may be acute heart failure. Additional cardiovascular diseases include, but are not limited to, congestive heart failure, compensated heart failure or decompensated heart failure. The disease or condition may be an autoimmune disorder. The autoimmune disorder may be scleroderma, diffuse scleroderma or systemic scleroderma. The disease or condition may be pain. The pain may be neuropathic pain or inflammatory pain. The disease or condition may be an inflammatory disease. The inflammatory disease may be fibromyalgia. The disease or condition may be fibrosis. Alternatively, the condition may be pregnancy. The modified therapeutic agent may be used to treat preeclampsia or induce labor. The modified therapeutic agent may be administered with one or more additional therapeutic agents. The additional therapeutic agents may comprise one or more of anti-inflammatory drugs, statins, diuretics, beta-blockers, angiotensin converting enzyme inhibitors and angiotensin II receptor blockers. The one or more additional therapeutic agents may be selected from the group consisting of an anti-inflammatory drug, a drug to treat pain, or any combination thereof. The additional therapeutic agent may be aspirin.

Further disclosed herein are methods for treating a disease or condition in a subject in need thereof, the method comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, and fatty alcohols; and (b) one or more therapeutic agents (TAs), wherein the one or more lipids are attached to at least one cysteine residue in the one or more therapeutic agents. The one or more TAs may comprise relaxin. The disease or condition may be a cardiovascular disease. The cardiovascular disease may be acute heart failure. Additional cardiovascular diseases include, but are not limited to, congestive heart failure, compensated heart failure or decompensated heart failure. The disease or condition may be an autoimmune disorder. The autoimmune disorder may be scleroderma, diffuse scleroderma or systemic scleroderma. The disease or condition may be an inflammatory disease. The inflammatory disease may be fibromyalgia. The disease or condition may be fibrosis. Alternatively, the condition is pregnancy. The LC may be used to treat preeclampsia or induce labor. The LC may be administered with one or more additional therapeutic agents. The additional therapeutic agents may comprise one or more of anti-inflammatory drugs, statins, diuretics, beta-blockers, angiotensin converting enzyme inhibitors and angiotensin II receptor blockers. The additional therapeutic agent may be aspirin.

Further disclosed herein are methods for treating a disease or condition in a subject in need thereof, the method comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, fatty amines, and fatty alcohols, and derivatives thereof; and (b) a therapeutic agent (TA), wherein TA is a peptide and the one or more lipids are attached to the peptide via an amino acid on the peptide. The TA may comprise relaxin. The disease or condition may be a cardiovascular disease. The cardiovascular disease may be acute heart failure. Additional cardiovascular diseases include, but are not limited to, congestive heart failure, compensated heart failure or decompensated heart failure. The disease or condition may be an autoimmune disorder. The autoimmune disorder may be scleroderma, diffuse scleroderma or systemic scleroderma. The disease or condition may be an inflammatory disease. The inflammatory disease may be fibromyalgia. The disease or condition may be fibrosis. Alternatively, the condition is pregnancy. The LC may be used to treat preeclampsia or induce labor. The LC may be administered with one or more additional therapeutic agents. The additional therapeutic agents may comprise one or more of anti-inflammatory drugs, statins, diuretics, beta-blockers, angiotensin converting enzyme inhibitors and angiotensin II receptor blockers. The additional therapeutic agent may be aspirin.

Further disclosed herein are methods of treating a disease or condition in a subject in need thereof, the method comprising administering to the subject one or more lipid conjugates (LCs) of Formula (II): TA-A¹-P¹-L, wherein TA is a therapeutic agent; A¹ is a chemical group linking TA and P¹; P¹ comprises a heteroaryl or heterocyclyl group; and L is a lipid. The TA may comprise relaxin. The disease or condition may be a cardiovascular disease. The cardiovascular disease may be acute heart failure. Additional cardiovascular diseases include, but are not limited to, congestive heart failure, compensated heart failure or decompensated heart failure. The disease or condition may be an autoimmune disorder. The autoimmune disorder may be scleroderma, diffuse scleroderma or systemic scleroderma. The disease or condition may be an inflammatory disease. The inflammatory disease may be fibromyalgia. The disease or condition may be fibrosis. Alternatively, the condition is pregnancy. The LC may be used to treat preeclampsia or induce labor. The LC may be administered with one or more additional therapeutic agents. The additional therapeutic agents may comprise one or more of anti-inflammatory drugs, statins, diuretics, beta-blockers, angiotensin converting enzyme inhibitors and angiotensin II receptor blockers. The additional therapeutic agent may be aspirin.

Provided herein is a method of preventing or treating a metabolic disease or condition in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, and fatty alcohols; (b) one or more therapeutic agents (TA); and (c) a linker connecting each of the one or more lipids to the TA, the linker comprising a heteroaryl or heterocyclyl group; wherein each linker is attached to one or more amino acid residues in the one or more therapeutic agents. The one or more TAs may comprise GLP-1, Exendin-4, exenatide, oxyntomodulin, glucagon, FGF21, or derivative thereof. The GLP-1 may be a human GLP-1. The FGF21 may be a human FGF21. The metabolic disease or condition may be diabetes. The metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating a metabolic disease or condition in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, fatty amines, and fatty alcohols, and derivatives thereof (b) a therapeutic agent (TA); and (c) a linker connecting each of the one or more lipids to the TA, the linker comprising a heteroaryl or heterocyclyl group; wherein the TA is a peptide and each linker is attached to the TA via an amino acid residue on the peptide. The TA may comprise GLP-1, Exendin-4, exenatide, oxyntomodulin, glucagon, FGF21, or derivative thereof. The GLP-1 may be a human GLP-1. The FGF21 may be a human FGF21. The metabolic disease or condition may be diabetes. The metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating a metabolic disease or condition in a subject in need thereof comprising administering to the subject one or more lipid conjugates (LCs) of Formula (II): TA-A¹-P¹-L, wherein TA is a therapeutic agent; A¹ is a chemical group linking TA and P¹; P¹ comprises a heteroaryl or heterocyclyl group; and L is a lipid. The TA may comprise GLP-1, exendin-4, exenatide, oxyntomodulin, glucagon, FGF21, or derivative thereof. The GLP-1 may be a human GLP-1. The FGF21 may be a human FGF21. The one or more lipids may comprise one or more sterols, sterol derivatives, bile acids, vitamin E derivatives, fatty di-acids, fatty acids, fatty amides, and fatty alcohols. The metabolic disease or condition may be diabetes. The metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating a central nervous system (CNS) disorder in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, and fatty alcohols; (b) one or more therapeutic agents (TAs); and (c) a linker connecting each of the one or more lipids to the TA, the linker comprising a heteroaryl or heterocyclyl group; wherein each linker is attached to at least one amino acid residue in the one or more therapeutic agents. The one or more TAs may comprise GLP-1, exendin-4, exenatide, oxyntomodulin, glucagon, or derivative thereof. The GLP-1 may be a human GLP-1. The CNS disorder may be Alzheimer's disease (AD). Additional CNS disorders include, but are not limited to, encephalitis, meningitis, tropical spastic paraparesis, arachnoid cysts, Huntington's disease, locked-in syndrome, Parkinson's disease, Tourette's, and multiple sclerosis.

Provided herein is a method of preventing or treating a central nervous system (CNS) disorder in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, fatty amines, and fatty alcohols, and derivatives thereof; (b) a therapeutic agent (TA); and (c) a linker connecting each of the one or more lipids to the TA, the linker comprising a heteroaryl or heterocyclyl group; wherein the TA is a peptide and each linker is attached to the TA via an amino acid residue on the peptide. The TA may comprise GLP-1, exendin-4, exenatide, oxyntomodulin, glucagon, or derivative thereof. The GLP-1 may be a human GLP-1. The CNS disorder may be Alzheimer's disease (AD). Additional CNS disorders include, but are not limited to, encephalitis, meningitis, tropical spastic paraparesis, arachnoid cysts, Huntington's disease, locked-in syndrome, Parkinson's disease, Tourette's, and multiple sclerosis.

Provided herein is a method of preventing or treating a central nervous system (CNS) disorder in a subject in need thereof comprising administering to the subject one or more lipid conjugates (LCs) of Formula (II): TA-A¹-P¹-L, wherein TA is a therapeutic agent; A¹ is a chemical group linking TA and P¹; P¹ comprises a heteroaryl or heterocyclyl group; and L is a lipid. The one or more lipids may comprise one or more sterols, sterol derivatives, bile acids, vitamin E derivatives, fatty di-acids, fatty acids, fatty amides, and fatty alcohols. The one or more lipids may comprise one or more sterols, sterol derivatives, bile acids, vitamin E derivatives, fatty di-acids, fatty acids, fatty amides, or fatty alcohols, or derivatives thereof. The TA may comprise GLP-1, exendin-4, exenatide, oxyntomodulin, glucagon, or derivative thereof. The GLP-1 may be a human GLP-1. The CNS disorder may be Alzheimer's disease (AD). Additional CNS disorders include, but are not limited to, encephalitis, meningitis, tropical spastic paraparesis, arachnoid cysts, Huntington's disease, locked-in syndrome, Parkinson's disease, Tourette's, and multiple sclerosis.

Provided herein is a method of preventing or treating a disease or condition which benefits from a GLP-1R and/or glucagon receptor (GCGR) agonist in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, and fatty alcohols; (b) one or more therapeutic agents (TAs); and (c) a linker connecting each of the one or more lipids to the TA, the linker comprising a heteroaryl or heterocyclyl group; wherein each linker is attached to at least one cysteine residue in the one or more therapeutic agents. The one or more TAs may comprise GLP-1, exendin-4, exenatide, oxyntomodulin, glucagon, or derivative thereof. The GLP-1 may be a human GLP-1. The disease or condition may be a metabolic disease or disorder. The disease or condition may be diabetes. The disease or condition may be obesity. Additional diseases and/or conditions which benefit from a GLP-1R and/or GCGR agonist include, but are not limited to, dyslipidemia, cardiovascular and fatty liver diseases.

Provided herein is a method of preventing or treating a disease or condition which benefits from a GLP-1R and/or glucagon receptor (GCGR) agonist in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, fatty amines, and fatty alcohols, and derivatives thereof; (b) a therapeutic agent (TA); and (c) a linker connecting each of the one or more lipids to the TA, the linker comprising a heteroaryl or heterocyclyl group; wherein the TA is a peptide and each linker is attached to the TA via an amino acid residue on the peptide. The TA may comprise GLP-1, exendin-4, exenatide, oxyntomodulin, glucagon, or derivative thereof. The GLP-1 may be a human GLP-1. The disease or condition may be a metabolic disease or disorder. The disease or condition may be diabetes. The disease or condition may be obesity. Additional diseases and/or conditions which benefit from a GLP-1R and/or GCGR agonist include, but are not limited to, dyslipidemia, cardiovascular and fatty liver diseases.

Provided herein is a method of preventing or treating a disease or condition which benefits from a GLP-1R and/or glucagon receptor (GCGR) agonist in a subject in need thereof comprising administering to the subject one or more lipid conjugates (LCs) of Formula (II): TA-A¹-P¹-L, wherein TA is a therapeutic agent; A¹ is a chemical group linking TA and P¹; P¹ comprises a heteroaryl or heterocyclyl group; and L is a lipid. The one or more lipids may comprise one or more sterols, sterol derivatives, bile acids, vitamin E derivatives, fatty di-acids, fatty acids, fatty amides, and fatty alcohols, or derivatives thereof. The one or more lipids may comprise one or more sterols, sterol derivatives, bile acids, vitamin E derivatives, fatty di-acids, fatty acids, fatty amides, or fatty alcohols, or derivatives thereof. The TA may comprise GLP-1, exendin-4, exenatide, oxyntomodulin, glucagon, or derivative thereof. The GLP-1 may be a human GLP-1. The disease or condition may be a metabolic disease or disorder. The disease or condition may be diabetes. The disease or condition may be obesity. Additional diseases and/or conditions which benefit from a GLP-1R and/or GCGR agonist include, but are not limited to, dyslipidemia, cardiovascular and fatty liver diseases.

Provided herein is a method of preventing or treating pain in a subject in need thereof comprising administering to the subject one or more LCs, wherein the one or more lipid conjugates (LCs) comprise (a) one or more lipids, the lipids selected from the group consisting of sterols, sterol derivatives, bile acids, vitamin E derivatives, fatty di-acids, fatty acids, fatty amides, fatty amines, and fatty alcohols, and derivatives thereof and (b) a therapeutic agent (TA); wherein the therapeutic agent is a peptide comprising at least one isosteric disulfide mimetic and the half-life of the modified therapeutic agent is longer than the half-life of the peptide alone. The pain may be neuropathic pain or inflammatory pain. The peptide may be a peptidyl toxin. The method may further comprise administering to the subject one or more additional therapeutic agents. The one or more additional therapeutic agents may be selected from the group consisting of an anti-inflammatory drug, a drug to treat pain, or any combination thereof. The one or more additional therapeutic agents may be aspirin.

### Compositions

Disclosed herein are pharmaceutical compositions comprising a modified therapeutic agent disclosed herein. The compositions may comprise 1, 2, 3, 4, 5, 6, 7, 8, 9, 10 or more modified therapeutic agents. The modified therapeutic agents may be different. Alternatively, the modified therapeutic agents may be the same or similar. The modified therapeutic agents may comprise different therapeutic agents, different half-life extending moieties, or a combination thereof. The half-life extending moieties may be the same or similar.

Further disclosed herein are pharmaceutical compositions comprising an LC disclosed herein. The compositions may comprise 1, 2, 3, 4, 5, 6, 7, 8, 9, 10 or more LCs. The LCs may be different. Alternatively, the LCs may be the same or similar. The LCs may comprise different therapeutic agents, different lipids, or a combination thereof. The lipids may be the same or similar.

The compositions described herein may further comprise one or more pharmaceutically acceptable salts, excipients, or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer can be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically may be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Pharmaceutical compositions described herein may be formulated for controlled or sustained delivery in a manner that provides local concentration of the product (e.g., bolus, depot effect) and/or increased stability or half-life in a particular local environment. The compositions can include the formulation of LCs, polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722.

Suitable materials for this purpose include polylactides (see, e.g., U.S. Pat. No. 3,773,919), polymers of poly-(α-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which can be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This can be determined by routine screening in animal models of the target disorder or, if such models are unavailable, in normal animals.

Microencapsulation of recombinant proteins for sustained release has been performed successfully with human growth hormone (rhGH), interferon-(rhIFN−), interleukin-2, and MN rgp120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids can be cleared quickly within the human body. Moreover, the degradability of this polymer can be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,481A, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha et al., J. Control. Release 90, 261 [2003], Zhu et al., Nat. Biotechnol. 18, 24 [2000], and Dai et al., Colloids Surf B Biointerfaces 41, 117 [2005].

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141).

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which an LC, nucleic acid encoding at least a portion of an LC, or vector comprising a nucleic acid encoding at least a portion of an LC disclosed herein has been absorbed or encapsulated. Where an implantation device is used, the device may be implanted into any suitable tissue or organ, and delivery of an LC, nucleic acid encoding at least a portion of an LC, or vector comprising a nucleic acid encoding at least a portion of an LC disclosed herein can be directly through the device via bolus, or via continuous administration, or via catheter using continuous infusion.

A pharmaceutical composition comprising an LC, nucleic acid encoding at least a portion of an LC, or vector comprising a nucleic acid encoding at least a portion of an LC disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations comprising an LC, nucleic acid encoding at least a portion of an LC, or vector comprising a nucleic acid encoding at least a portion of an LC disclosed herein may be administered orally. Formulations administered in this fashion may be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also can be employed.

Another preparation may involve an effective quantity of an antibody comprising an LC, nucleic acid encoding at least a portion of an LC, or vector comprising a nucleic acid encoding at least a portion of an LC disclosed herein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable salt” refers to a salt of a compound that is pharmaceutically acceptable and that possesses the desired pharmacological activity of the parent compound.

“Pharmaceutically acceptable excipient, carrier or adjuvant” refers to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” refers to a diluent, adjuvant, excipient, or carrier with which at least one antibody of the present disclosure is administered.

### Vectors, Host Cells and Recombinant Methods

A TA, as disclosed herein, may be expressed by recombinant methods. Generally, a nucleic acid encoding a TA may be isolated and inserted into a replicable vector for further cloning (amplification of the DNA) or for expression. DNA encoding the LC may be prepared by PCR amplification and sequenced using conventional procedures (e.g., by using oligonucleotide probes that are capable of binding specifically to nucleotides encoding LC). In an exemplary embodiment, a nucleic acid encoding an LC is PCR amplified, restriction enzyme digested and gel purified. The digested LC may be inserted into a replicable vector. The replicable vector containing the digested LC insertion may be transformed or transduced into a host cell for further cloning (amplification of the DNA) or for expression. Host cells may be prokaryotic or eukaryotic cells.

Polynucleotide sequences encoding polypeptide components of the LCs disclosed herein may be obtained by PCR amplification with overlapping oligonucleotide primers. Polynucleotide sequences may be isolated and sequenced from TA producing cells. Alternatively, polynucleotides may be synthesized using nucleotide synthesizer or PCR techniques. Once obtained, sequences encoding the polypeptides are inserted into a recombinant vector capable of replicating and expressing heterologous polynucleotides in prokaryotic and/or eukaryotic hosts.

In addition, phage vectors containing replicon and control sequences that are compatible with the host microorganism may be used as transforming vectors in connection with these hosts. For example, bacteriophage such as λGEM™-11 may be utilized in making a recombinant vector which can be used to transform susceptible host cells such as E. coli LE392.

TAs may be expressed intracellularly (e.g., cytoplasm) or extracellularly (e.g., secretion). For extracellular expression, the vector may comprise a secretion signal which enables translocation of the TA to the outside of the cell.

Suitable host cells for cloning or expression of TA-encoding vectors include prokaryotic or eukaryotic cells. The host cell may be a eukaryotic. Examples of eukaryotic cells include, but are not limited to, Human Embryonic Kidney (HEK) cell, Chinese Hamster Ovary (CHO) cell, fungi, yeasts, invertebrate cells (e.g., plant cells and insect cells), lymphoid cell (e.g., YO, NSO, Sp20 cell). Other examples of suitable mammalian host cell lines are monkey kidney CV1 line transformed by SV40 (COS-7); baby hamster kidney cells (BHK); mouse Sertoli cells; monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (HepG2); mouse mammary tumor (MMT 060562); TR1 cells; MRC 5 cells; and FS4 cells. The host cell may be a prokaryotic cell (e.g., E. coli).

Host cells may be transformed with vectors containing nucleotides encoding a TA. Transformed host cells may be cultured in media. The media may be supplemented with one or more agents for inducing promoters, selecting transformants, or amplifying or expressing the genes encoding the desired sequences. Methods for transforming host cells are known in the art and may include electroporation, calcium chloride, or polyethylene glycol/DMSO.

Alternatively, host cells may be transfected or transduced with vectors containing nucleotides encoding a TA. Transfected or transduced host cells may be cultured in media. The media may be supplemented with one or more agents for inducing promoters, selecting transfected or transduced cells, or expressing genes encoding the desired sequences.

The expressed TAs may be secreted into and recovered from the periplasm of the host cells or transported into the culture media. Protein recovery from the periplasm may involve disrupting the host cell. Disruption of the host cell may comprise osmotic shock, sonication or lysis. Centrifugation or filtration may be used to remove cell debris or whole cells. The TAs may be further purified, for example, by affinity resin chromatography.

Alternatively, TAs that are secreted into the culture media may be isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

TA production may be conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors may use agitator impellers to distribute oxygen and nutrients, especially glucose (a preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described herein. Cells may be grown for shorter periods prior to induction. Cells are usually induced for about 12-50 hours, although longer or shorter induction times may be used.

To improve the production yield and quality of the TAs disclosed herein, various fermentation conditions can be modified. For example, to improve the proper assembly and folding of the secreted TA polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) may be used to co-transform the host prokaryotic cells. The chaperone proteins have been demonstrated to facilitate the proper folding and solubility of heterologous proteins produced in bacterial host cells.

To minimize proteolysis of expressed heterologous proteins (especially those that are proteolytically sensitive), certain host strains deficient for proteolytic enzymes can be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available.

Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, hydroxylapatite chromatography, gel electrophoresis, dialysis, and affinity chromatography and gel filtration using, for example, Sephadex G-75.

TAs may be concentrated using a commercially available protein concentration filter, for example, an Amicon or Millipore Pellicon® ultrafiltration unit.

Protease inhibitors or protease inhibitor cocktails may be included in any of the foregoing steps to inhibit proteolysis of the TA.

In some cases, a TA or fragment thereof may not be biologically active upon isolation. Various methods for “refolding” or converting a polypeptide to its tertiary structure and generating disulfide linkages, can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. The refolding/oxidation solution may also contain a reducing agent or the reducing agent plus its oxidized form in a specific ratio to generate a particular redox potential allowing for disulfide shuffling to occur in the formation of the protein's cysteine bridge(s). Some of the commonly used redox couples include cystein/cystamine, glutathione (GSH)/dithiobis GSH, cupric chloride, dithiothreitol (DTT)/dithiane DTT, and 2-mercaptoethanol (bME)/di-thio-b (ME). In many instances, a cosolvent may be used to increase the efficiency of the refolding, and common reagents used for this purpose include glycerol, polyethylene glycol of various molecular weights, arginine and the like. Choice of buffer may determine the three-dimensional structure of the refolded peptide. Use of a buffer comprising ammonium sulfate may result in the desired refolded peptide in better yield than other types of buffers.

### Kits/Articles of Manufacture

As an additional aspect, the present disclosure includes kits which comprise one or more compounds or compositions packaged in a manner which facilitates their use to practice methods of the present disclosure. In one embodiment, such a kit includes a compound or composition described herein (e.g., a modified therapeutic agent alone or in combination with a second agent), packaged in a container with a label affixed to the container or a package insert that describes use of the compound or composition in practicing the method. Suitable containers include, for example, bottles, vials, syringes, etc. The containers may be formed from a variety of materials such as glass or plastic. The container may have a sterile access port (for example the container may be an intravenous solution bag or a vial having a stopper pierceable by a hypodermic injection needle). The article of manufacture may comprise (a) a first container with a composition contained therein, wherein the composition comprises a modified therapeutic agent as disclosed herein; and (b) a second container with a composition contained therein, wherein the composition comprises a further therapeutic agent. The article of manufacture in this embodiment disclosed herein may further comprise a package insert indicating that the first and second compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. Preferably, the compound or composition is packaged in a unit dosage form. The kit may further include a device suitable for administering the composition according to a specific route of administration or for practicing a screening assay. Preferably, the kit contains a label that describes use of the modified therapeutic agent composition.

In certain embodiments, the composition comprising the modified therapeutic agent is formulated in accordance with routine procedures as a pharmaceutical composition adapted for intravenous administration to mammals, such as humans, bovines, felines, canines, and murines. Typically, compositions for intravenous administration are solutions in sterile isotonic aqueous buffer. Where necessary, the composition may also include a solubilising agent and a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the composition described herein which may be effective in the treatment, inhibition and prevention of a disease or disorder associated with aberrant expression and/or activity of a protein can be determined by standard clinical techniques. In addition, in vitro assays may optionally be employed to help identify optimal dosage ranges. The precise dose to be employed in the formulation may also depend on the route of administration, and the seriousness of the disease or disorder, and should be decided according to the judgment of the practitioner and each patient's circumstances. Effective doses are extrapolated from dose-response curves derived from in vitro or animal model test systems.

### Definitions

The terms below, as used herein, have the following meanings, unless indicated otherwise:

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a straight or branched hydrocarbon chain radical, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —ORₐ where Rₐ is an alkyl radical as defined. Unless stated otherwise specifically in the specification, an alkoxy group may be optionally substituted as described below.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Cycloalkyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted. Illustrative examples of cycloalkyl groups include, but are not limited to, the following moieties:

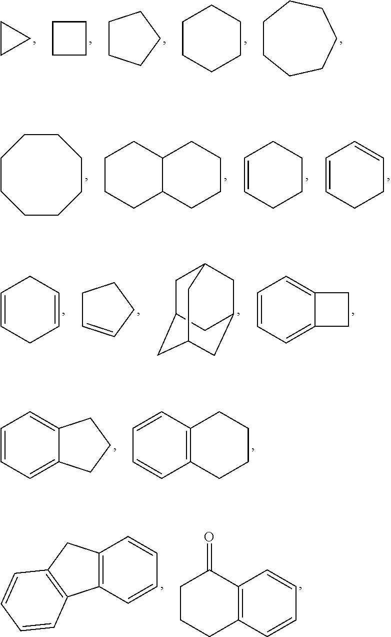

and the like.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —ORₐ where Rₐ is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted. Illustrative examples of heterocycloalkyl groups, also referred to as non-aromatic heterocycles, include:

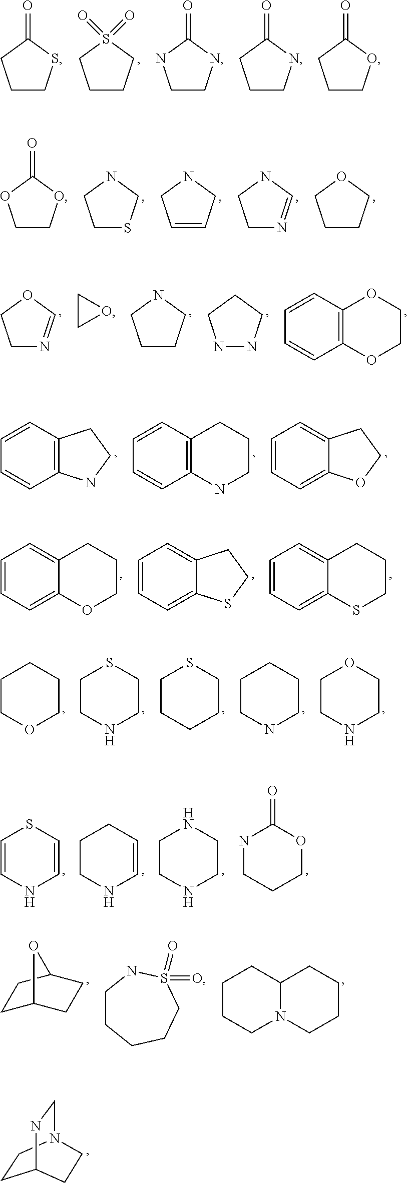

and the like. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, azepinyl, phenazinyl, benzimidazolyl, benzindolyl, benzofuranyl, benzofuranonyl, benzoxazolyl, benzothiazolyl, benzothiadiazolyl, benzothiophenyl, benzoxadiazolyl, benzodioxolyl, benzo[b][1,4]dioxepinyl, 1,4-benzodioxanyl, benzonaphthofuranyl, benzotriazolyl, benzodioxolyl, benzodioxinyl, benzopyranyl, benzopyranonyl, benzothienyl (benzothiophenyl), benzo[4,6]imidazo[1,2-a]pyridinyl, carbazolyl, cinnolinyl, dibenzofuranyl, dibenzothiophenyl, furanonyl, imidazolyl, indolyl, isoxazolyl, isoquinolinyl, indolizinyl, indazolyl, isoindolyl, indolinyl, isoindolinyl, indolizinyl, isothiazolyl, isoindolyloxadiazolyl, indazolyl, naphthyridinyl, oxadiazolyl, 2-oxoazepinyl, oxiranyl, 1-oxidopyridinyl, 1-oxidopyrimidinyl, 1-oxidopyrazinyl, 1-oxidopyridazinyl, 1-phenyl-1H-pyrrolyl, phenothiazinyl, phenoxazinyl, pyridinyl, pyridazinyl, pyrimidinyl, pyrazinyl, pyrrolyl, pyrazinyl, pyrazolyl, purinyl, phthalazinyl, pteridinyl, quinolinyl, quinazolinyl, quinoxalinyl, quinuclidinyl, triazolyl, tetrazolyl, thiazolyl, triazinyl, thiadiazolyl, tetrahydroquinolinyl, thiazolyl, and thiophenyl and the like, and their oxides, such as for example pyridyl-N-oxide. Illustrative examples of heteroaryl groups include the following moieties:

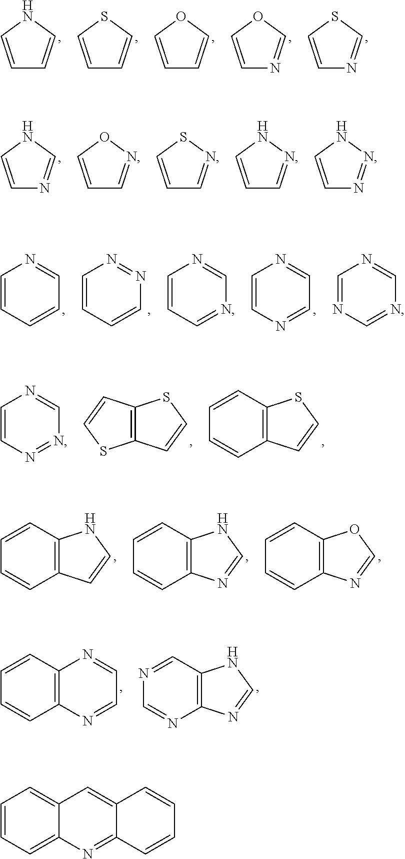

and the like.

All the above groups may be either substituted or unsubstituted. The term “substituted” as used herein means any of the above groups (e.g., alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO₂H, nitrile, nitro, hydroxyl, thiooxy, alkyl, alkylene, alkoxy, aryl, cycloalkyl, heterocyclyl, heteroaryl, di alkylamines, arylamines, alkylarylamines, diarylamines, trialkylammonium (—N⁺R₃), N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NH₂, —NR_{g}C(═O)NR_{g}Rₕ, —NR_{g}C(═O)ORₕ, —NR_{g}SO₂Rₕ, —OC(═O)NR_{g}Rₕ, —OR_{g},

—SR_{g}, —SOR_{g}, —SO₂R_{g}, —OSO₂R_{g}, —SO₂OR_{g}, ═NSO₂R_{g}, and —SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarly, an alkyl group may be substituted with one or more internal sulfur atoms to form a thioether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

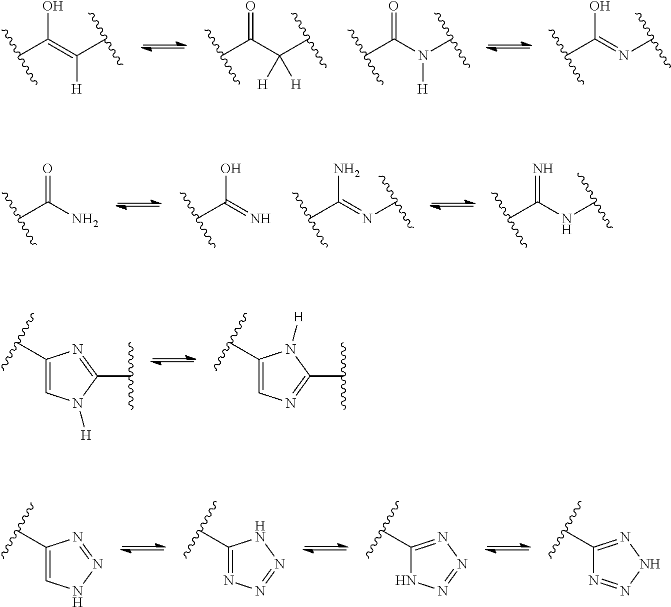

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. Metabolites of a compound may be formed by oxidative processes and correspond to the corresponding hydroxy-containing compound. A compound may be metabolized to one or more pharmacologically active metabolites.

As used herein, a “derivative” of a peptide refers to, but is not limited to, a modified peptide that allows for lipid attachment (such as one or more amino acid residue replacements or L- vs D-amino acid replacements), a fragment, an analog with one or more additional amino acids, a complex and/or an aggregate of the peptide. A derivative of a peptide may be a homolog that has at least 50% homology with respect to the peptide. A derivative of a peptide may be a homolog that has at least 60% homology with respect to the peptide. A derivative of a peptide may be a homolog that has at least 70% homology with respect to the peptide. A derivative of a peptide may be a homolog that has at least 80% homology with respect to the peptide. A derivative of a peptide may be a homolog that has at least 90% homology with respect to the peptide.

The terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

Terms such as “treating” or “treatment” or “to treat” or “alleviating” or “to alleviate” may refer to: 1) therapeutic measures that cure, slow down, lessen symptoms of, and/or halt progression of a diagnosed pathologic condition or disorder; and/or 2) prophylactic or preventative measures that prevent and/or slow the development of a targeted pathologic condition or disorder. Thus those in need of treatment include those already with the disorder; those prone to have the disorder; and those in whom the disorder is to be prevented.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

As used herein, the term “therapeutic agent” or “peptide therapeutic agent” refers to a protein or peptide that modulates the activity of another protein, peptide, cell or tissue. Modulating the activity can comprise increasing, decreasing, stimulating, or preventing the activity or expression of the protein, peptide, cell or tissue. Therapeutic agents may modulate the activity of proteins or peptides involved in the etiology of a disease or disorder. Exemplary TAs may include, but are not limited to, at least a portion of a hormone, kinase, receptor, ligand, growth factor, regulatory protein, metabolic protein, cytokine, chemokine, interferon, phosphatase, antibody or any combination thereof.

“Disorder” or “disease” refers to a condition that would benefit from treatment with a substance/molecule (e.g., a modified therapeutic agent as disclosed herein) or method disclosed herein. This includes chronic and acute disorders or diseases including those pathological conditions which predispose the mammal to the disorder in question.

“Treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

“Mammal” for purposes of treatment refers to any animal classified as a mammal, including humans, rodents (e.g., mice and rats), and monkeys; domestic and farm animals; and zoo, sports, laboratory, or pet animals, such as dogs, cats, cattle, horses, sheep, pigs, goats, rabbits, etc. In some embodiments, the mammal is selected from a human, rodent, or monkey.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable salt” refers to a salt of a compound that is pharmaceutically acceptable and that possesses the desired pharmacological activity of the parent compound.

“Pharmaceutically acceptable excipient, carrier or adjuvant” refers to an excipient, carrier or adjuvant that can be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” refers to a diluent, adjuvant, excipient, or carrier with which at least one antibody of the present disclosure is administered.

The terms “modified therapeutic agent,”” “lipid conjugate,” and “LC” may be used interchangeably. These terms may refer to a therapeutic agent (TA) attached to a half-life extending moiety.

### Examples

### Example 1. Gene Construction and Expression of Relaxin Analogs

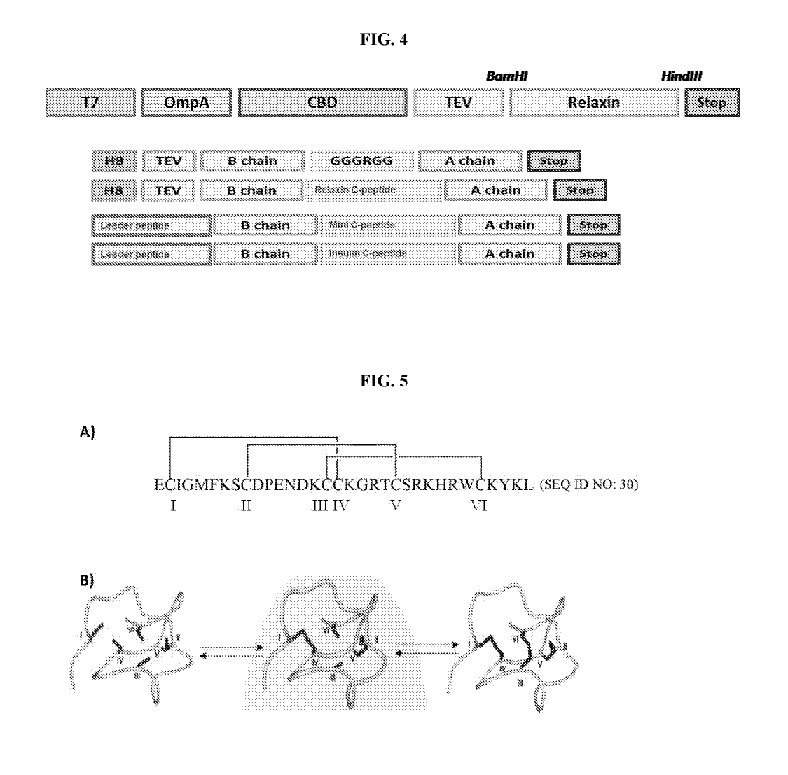

The inserts were prepared through polymerase cycling assembly, where forward and reverse primers were annealed to generate the DNA fragment which was further enriched by forward and reverse end primers. The amplified fragment was then digested with restriction digestion enzymes (NdeI/KpnI). The prepared insert was then ligated into a vector with a PET backbone under the control of a T7 promoter and terminator (e.g., see FIG. 4). The encoded DNA fragment was then confirmed by DNA sequencing. For expression, relaxin constructs were transformed into K12 E. coli (derivative of W3110) with a genomic T7 RNA polymerase gene under the control of a araBAD promoter. The transformed E. coli cells were cultured in LB supplemented with 50 μg/ml kanamycin to OD600 nm=0.8 at 37° C. with vigorous shaking (250 rpm). To induce expression of the genes, L-arabinose stock solution (20% w/v in H₂O) was added to the final concentration of 0.2%, and the cells were continuously incubated with shaking at 250 rpm at 37° C. for 5 h. Cells were then harvested by centrifugation (6000 rpm, 15 min). Preparation of inclusion bodies was performed as described in Example 18.

### Example 2. Measuring Bioactivity of Relaxin Peptides

Relaxin 2 signals through leucine-rich repeat-containing GPCRs (i.e LGR7 (RFXR1)). The bioactivity of wild-type relaxin peptides was determined based on the stimulation of adenylate cyclase activity in HEK293T cells stably expressing recombinant LGR7. Stable LGR7 expressing cells were maintained in Dulbecco's modified Eagle's medium/F-12 media supplemented with 200 ng/ml Zeocin (Invitrogen). A cyclic AMP responsive element driven luciferase (CRE-Luc) reporter line was generated by lentiviral transduction of these LGR7 expressing HEK293T cells with CRE-Luc lentivirus (Qiagen) and selected with puromycine (2 μg/mL) for two weeks. A reporter gene assay was used to detect cAMP levels and LGR7 activation by relaxin.

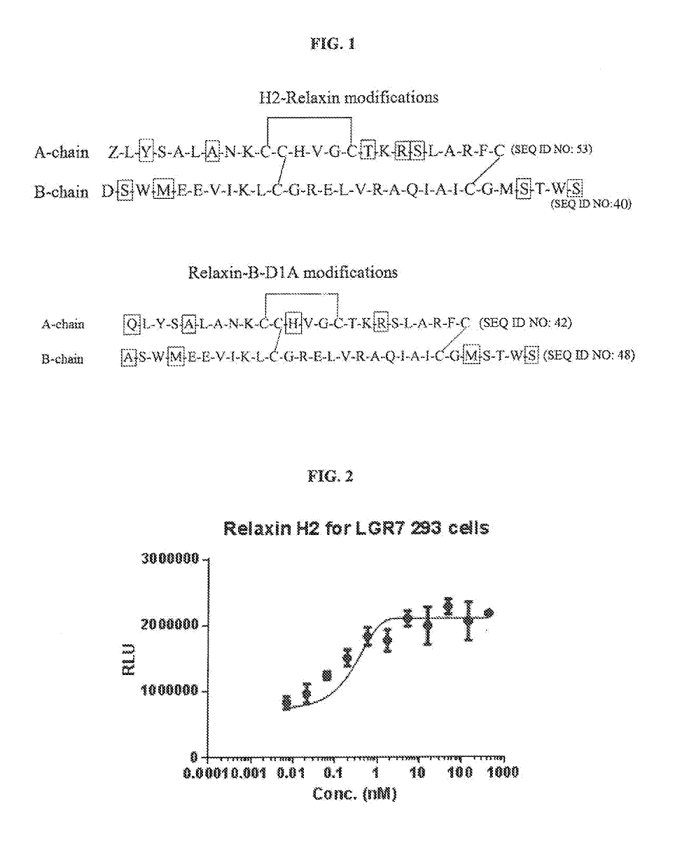

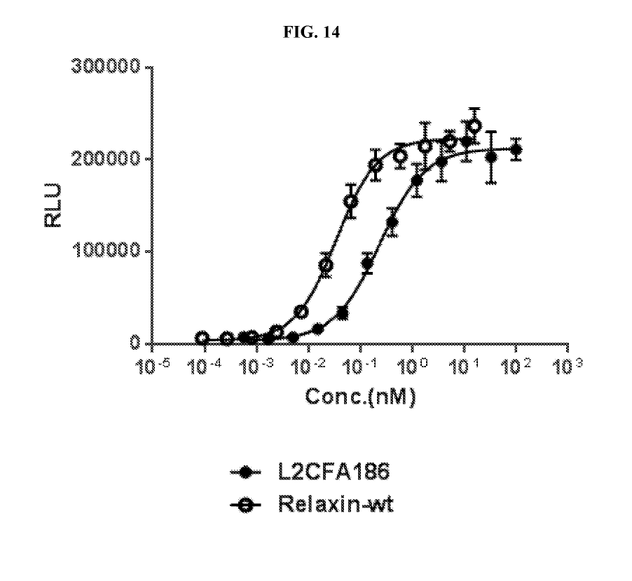

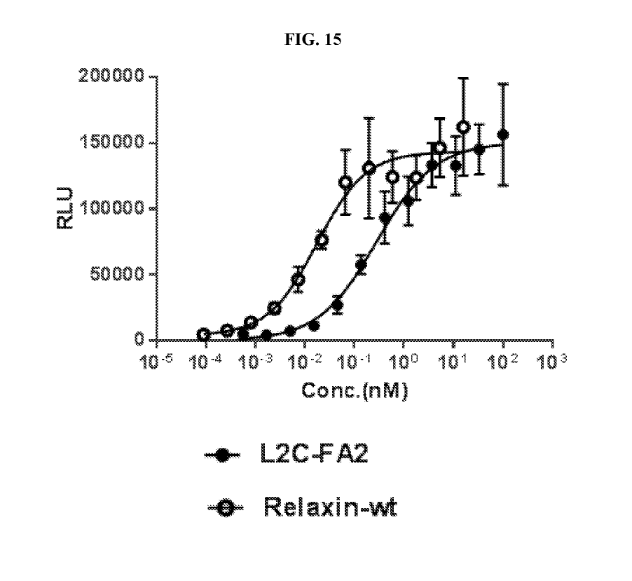

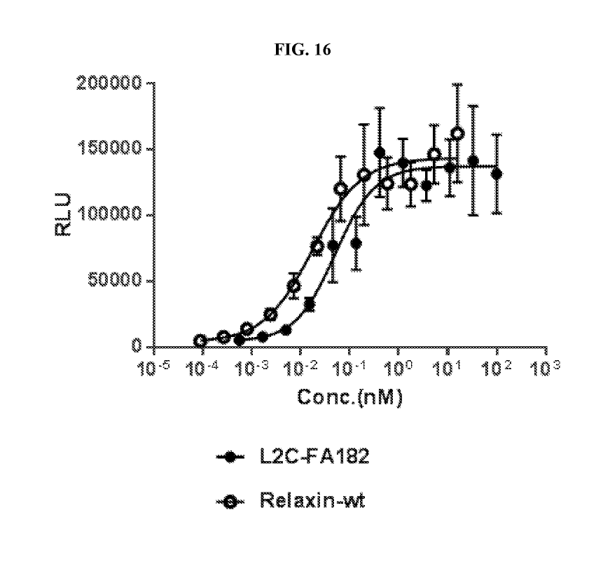

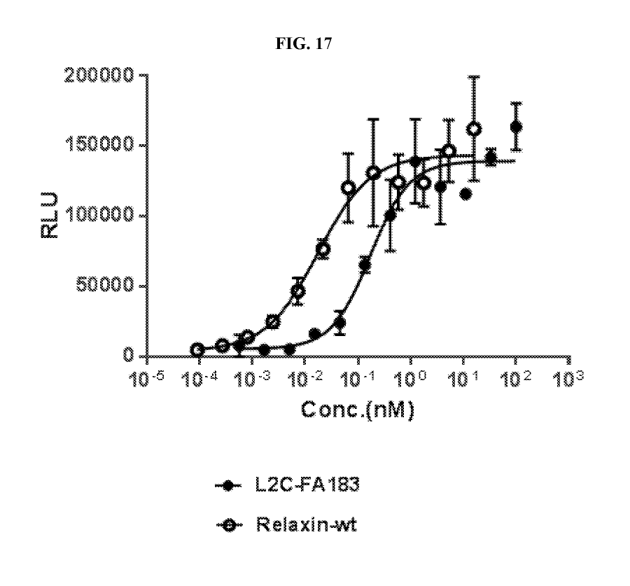

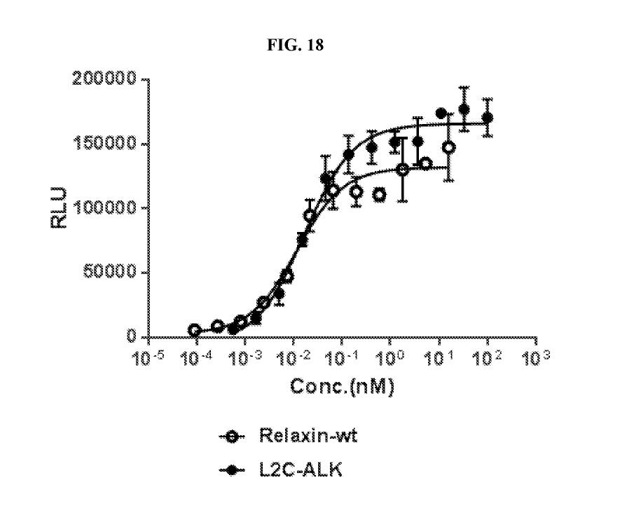

Assay detail: HEK293T cells expressing LGR7 and CRE-Luc are seeded at a density of 5×10³ cells per well in 50 μL of Dulbecco's modified Eagle's medium/F-12 medium containing 10% FBS in 384-well solid bottom white plates. Cells are pre-incubated at 37° C. overnight. Different concentrations of relaxin peptides (from 0.01 nM to 1000 nM) were added in triplicate, incubated for 18 hours and luciferase activity was detected by adding 104, of Bright Glo (Promega). Luminescence was recorded on Envision (Perkin Elmer). EC₅₀ is calculated after non-linear curve fitting (see FIG. 2 for relaxin H2 dose response curve). Select data is shown in Table 3 and FIG. 14-18.

### Example 3. Synthesis of Pegylated Lipid (Amide Linkage) Conjugated Relaxin (Scheme A)

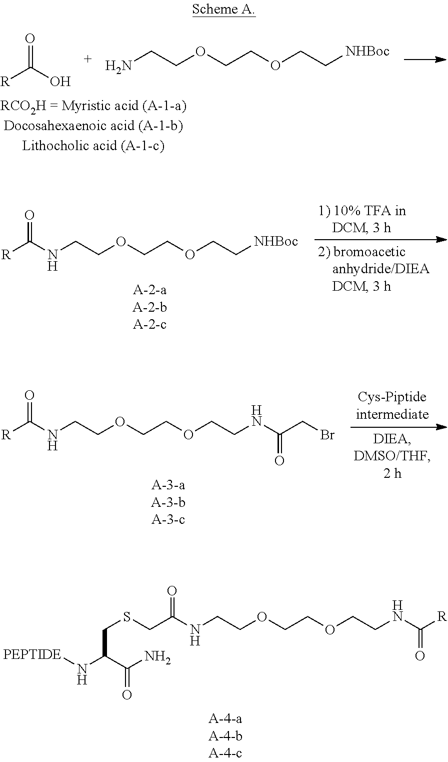

### tert-Butyl (2-(2-(2-tetradecanamidoethoxy)ethoxy)ethyl)carbamate (A-2-a)

N-t-Boc-amido-dPEG3-amine (0.5 g, 2.0 mmol) was added to a solution of myristic acid (0.46 g, 2.0 mmol) in 10 ml of dry DMF, followed by HATU (0.8 g, 2.1 mmol) and DIPEA (0.45 mL, 2.4 mmol). The mixture was stirred at RT for 6 h and the solvent was evaporated in vacuo. The crude material was dissolved in EtOAc, washed with cold 1% HCl, saturated NaHCO₃ and brine, dried over Na₂SO₄, filtered and concentrated. The crude material was purified by flash column chromatography on silica gel with a gradient 25-50% EtOAc in hexanes to afford 0.83 g of desired compound as white solids (Yield 90%). m/z (ESI+) 459.6 (M+H).

### tert-Butyl (2-(2-(2-((4Z,7Z,10Z,13Z,16Z,19Z)-docosa-4,7,10,13,16,19-hexaenamido)ethoxy)ethoxy)ethyl)carbamate (A-2-b)

The title compound was prepared using analogous conditions as the procedure for A-2-a, using N-t-Boc-amido-dPEG3-amine (0.25 g, 1.0 mmol), docosahexaenoic acid (0.33 g, 1.0 mmol), HATU (0.41 g, 1.1 mmol) and DIPEA (0.22 mL, 1.2 mmol in dry DMF (5 mL). Yield 68%, brown oil. ¹H NMR (500 MHz; CDCl₃): δ 0.97 (t, J=6.0 Hz, 3H), 1.44 (s, 9H), 2.07 (t, J=5.0 Hz, 2H), 2.25 (J=5.0 Hz, 2H), 2.41 (dd, J=5.0, 6.5 Hz, 2H), 2.79-2.87 (m, 10H), 3.32 (t, J=5.5 Hz, 2H), 3.45 (dd, J=5.0, 6.0 Hz, 2H), 3.55 (t, J=5.0 Hz, 4H), 3.59-3.62 (m, 4H), 4.97 (s, 1H), 5.30-5.42 (m, 12H), 6.03 (s, 1H); m/z (ESI+) 459.6 (M+H).

### tert-Butyl (2-(2-(2-((R)-4-((3R,5R,8R,9S,10S,13R,14S,17R)-3-hydroxy-10,13-dimethylhexadecahydro-1H-cyclopenta[a]phenanthren-17-yl)pentanamido)ethoxy)ethoxy) ethyl)carbamate (A-2-c)

N-t-Boc-amido-dPEG3-amine (0.14 g, 0.55 mmol) was added to a solution of NHS-activated lithocholic acid ester (0.24 g, 0.5 mmol) in 5 ml of dry DMF, followed by DIPEA (0.18 mL, 1.0 mmol). The mixture was stirred at RT for 16 h, and the solvent was evaporated in vacuo. The crude material is dissolved in DCM, washed with cold 1% HCl, saturated NaHCO₃ and brine, dried over Na₂SO₄, filtered and concentrated. The crude material was purified by flash column chromatography on silica gel with a gradient 1-5% methanol in DCM to afford 0.48 g of desired compound as a white solid (Yield 80%). ¹H NMR (500 MHz; CDCl₃): δ 0.62 (s, 3H), 0.90-2.25 (m, 30H), 3.31 (s, 2H), 3.43-3.46 (s, 2H), 3.52-3.56 (m, 4H), 3.59-3.60 (m, 6H), 4.94 (s, 1H), 6.05 (s, 1H); m/z (ESI+) 628.6 (M+H).m/z (ESI+) 607.5 (M+H).

### N-(2-(2-(2-(2-Bromoacetamido)ethoxy)ethoxy)ethyl)tetradecanamide (A-3-a)

TFA (2 ml, 26 mmol) was added to a solution of A-2-a (0.46 g, 1 mmol) in 10 ml of DCM, and the mixture was stirred at RT for 3 h. The reaction mixture was concentrated, and the crude material was lyophilized to obtain a colorless oil that was dissolved in 10 ml of DCM. Bromoacetic anhydride (0.31 g, 1.2 mmol) was added, followed by DIPEA (0.52 ml, 2.5 mmol), and the mixture was stirred at RT for 3 h. The reaction mixture was extracted with DCM and EtOAc, washed with 1% HCl, saturated NaHCO₃ and brine, dried over Na₂SO₄, filtered and concentrated. The crude material was purified by flash column chromatography on silica gel with a gradient 20-50% EtOAc in petroleum ether with 5% methanol to obtain 0.37 g of desired compound as a white solid (combined yield over two steps, 78%). ¹H NMR (500 MHz; CDCl₃): δ 0.88 (t, J=6.5 Hz, 3H), 1.25-1.32 (m, 20H), 1.62 (t, J=7.5 Hz, 2H), 2.18 (t, J=8.0 Hz, 2H), 3.47 (dd, J=5.0, 10.0 Hz, 2H), 3.50 (dd, J=5.0, 10.0 Hz, 2H), 3.56-3.58 (m, 2H), 3.59-3.62 (m, 2H), 3.63 (d, J=5.5 Hz, 5H), 3.88 (s, 2H), 5.92-5.93 (m, 1H), 6.94 (s, 1H); 7.04-7.17 (m, 1H); 13C NMR (100 MHz; CDCl3): δ 14.55, 23.12, 26.20, 29.76, 29.79, 29.95, 30.06, 30.08, 30.09, 30.11, 32.35, 37.23, 69.84, 70.46, 70.83 (2), 166.06, 173.84; m/z (ESI+) 480.6 (M+H).

### (4Z,7Z,10Z,13Z,16Z,19Z)—N-(2-(2-(2-(2-bromoacetamido)ethoxy)ethoxy)ethyl) docosa-4,7,10,13,16,19-hexaenamide (A-3-b)

The title compound was prepared using analogous conditions as the procedure for A-3-a. Yield 67%, brown oil. ¹H NMR (500 MHz; CDCl₃): δ 0.96 (t, J=5.0 Hz, 3H), 2.07 (q, J=7.6 Hz, 2H), 2.23 (J=7.2 Hz, 2H), 2.40 (dd, J=7.2, 13.8 Hz, 2H), 2.79-2.84 (m, 10H), 3.44-3.51 (m, 4H), 3.55-3.62 (m, 4H), 3.63 (s, 4H), 3.88 (s, 2H), 5.78-5.42 (m, 12H), 5.94 (s, 1H), 6.92 (s, 1H); m/z (ESI+) 559.8 (M+H).

### (R)—N-(2-(2-(2-(2-bromoacetamido)ethoxy)ethoxy)ethyl)-4-((3R,5R,8R,9S,10S,13R,14S,17R)-3-hydroxy-10,13-dimethylhexadecahydro-1H-cyclopenta[a]phenanthren-17-yl)pentanamide (A-3-c)

The title compound was prepared using analogous conditions as the procedure for A-3-a, replacing A-2-a with A-2-c. Yield 87%, white solid. ¹H NMR (500 MHz; CDCl₃): δ 0.83 (t, J=4.0 Hz, 3H), 0.85-0.94 (m, 6H), 1.00-1.91 (m, 28H), 3.41-3.64 (m, 12H), 3.85-3.88 (m, 2H), 4.86-4.94 (m, 1H), 6.05 (t, J=7.2 Hz, 1H), 7.04 (s, 1H); m/z (ESI+) 628.6 (M+H).

### Synthesis of Peptides Derivatized with Fatty Acid or Bile Acid (A-4-a, A-4-b, or A-4-c)

The Cys peptide intermediate (1 equiv) is dissolved in DMSO and is reacted with any one of A-3-a, A-3-b, or A-3-c (2 equiv) dissolved in THF, followed by addition of DIPEA (3% by volume). Reaction completion is assessed by HPLC-MS. The reaction is quenched by addition of TFA to a final pH of 4, and directly purified by preparative reverse-phase HPLC using eluents (A) 0.05% TFA in water and (B) 0.05% TFA in acetonitrile, and the following gradient for eluent (B): 10% (1 min) 40-55% (10 min), flow rate 80 ml/min. The purified peptides are lyophilized, and structure and purity are confirmed by analytical HPLC and electrospray mass spectrometry.

### Example 4. Synthesis of Pegylated Lipid (Ether Linkage) Conjugated Relaxin (Schemes B-1 and B-2)

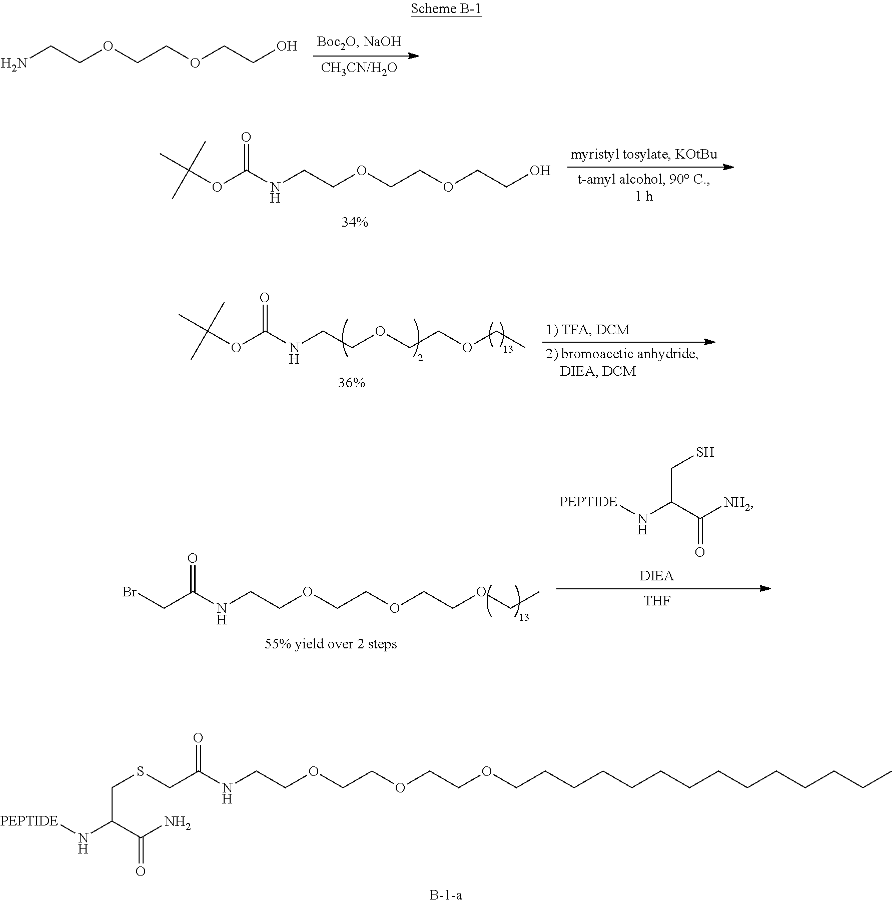

A solution of 2-(2-(2-aminoethoxy)ethoxy)ethanol (100 mg, 0.671 mmol, 89 μL) in 4.8 mL of acetonitrile/water (6:1) was treated with di-tert-butyl dicarbonate (151 mg, 0.691 mmol), followed by 0.5 mL of 1 N NaOH (aq). After stirring at RT for 45 min, the organic solvent was removed in vacuo, the residue was dissolved in saturated NH₄Cl (aq), and the desired carbamate was extracted with EtOAc. Removal of EtOAc provided 57 mg (34% yield) of tert-butyl 2-(2-(2-hydroxyethoxy)ethoxy)ethylcarbamate as a colorless oil.

A solution of tert-butyl 2-(2-(2-hydroxyethoxy)ethoxy)ethylcarbamate (68.5 mg, 0.275 mmol) and myristyl tosylate (101 mg, 0.275 mmol) in 1.4 mL of t-amyl alcohol was treated with potassium tert-butoxide (61.6 mg, 0.550 mmol) and potassium iodide (4.6 mg, 0.028 mmol). After heating to 90° C. for 2 h, the reaction was allowed to cool to rt, quenched with saturated NH₄Cl (aq), then extracted with EtOAc and dried over Na₂SO₄. Concentration and subsequent purification via flash column chromatography on silica gel afforded 44 mg (36% yield) of tert-butyl 2-(2-(2-(tetradecyloxy)ethoxy)ethoxy)ethylcarbamate as a colorless oil.

A solution of tert-butyl 2-(2-(2-(tetradecyloxy)ethoxy)ethoxy)ethylcarbamate (26 mg, 0.058 mmol) in 1.2 mL of DCM was treated with 0.29 mL of trifluoroacetic acid. After stirring at rt for 50 min, the mixture was concentrated and re-dissolved in 2 mL DCM. A 1-mL aliquot of this solution was taken into a separate vial, which was cooled to 0° C. and subsequently charged with bromoacetic anhydride (10.3 mg, 0.040 mmol) and DIPEA (11 μL, 0.063 mmol). After stirring at rt for 12 h, the reaction mixture was purified via flash column chromatography to provide 7.5 mg (55% yield over 2 steps) of 2-bromo-N-(2-(2-(2-(tetradecyloxy)ethoxy)ethoxy)-ethyl)acetamide as an off-white solid.

A solution of the Cys-peptide intermediate (1 equiv) in DMSO (4 mM) is treated with 2-bromo-N-(2-(2-(2-(tetradecyloxy)ethoxy)ethoxy)-ethyl)acetamide (2.1 equiv) in THF (5.4 mM) and DIPEA (114 equiv). Upon reaction completion, the reaction mixture is purified by preparative reverse-phase HPLC to provide the lipid conjugate (B-1-a).

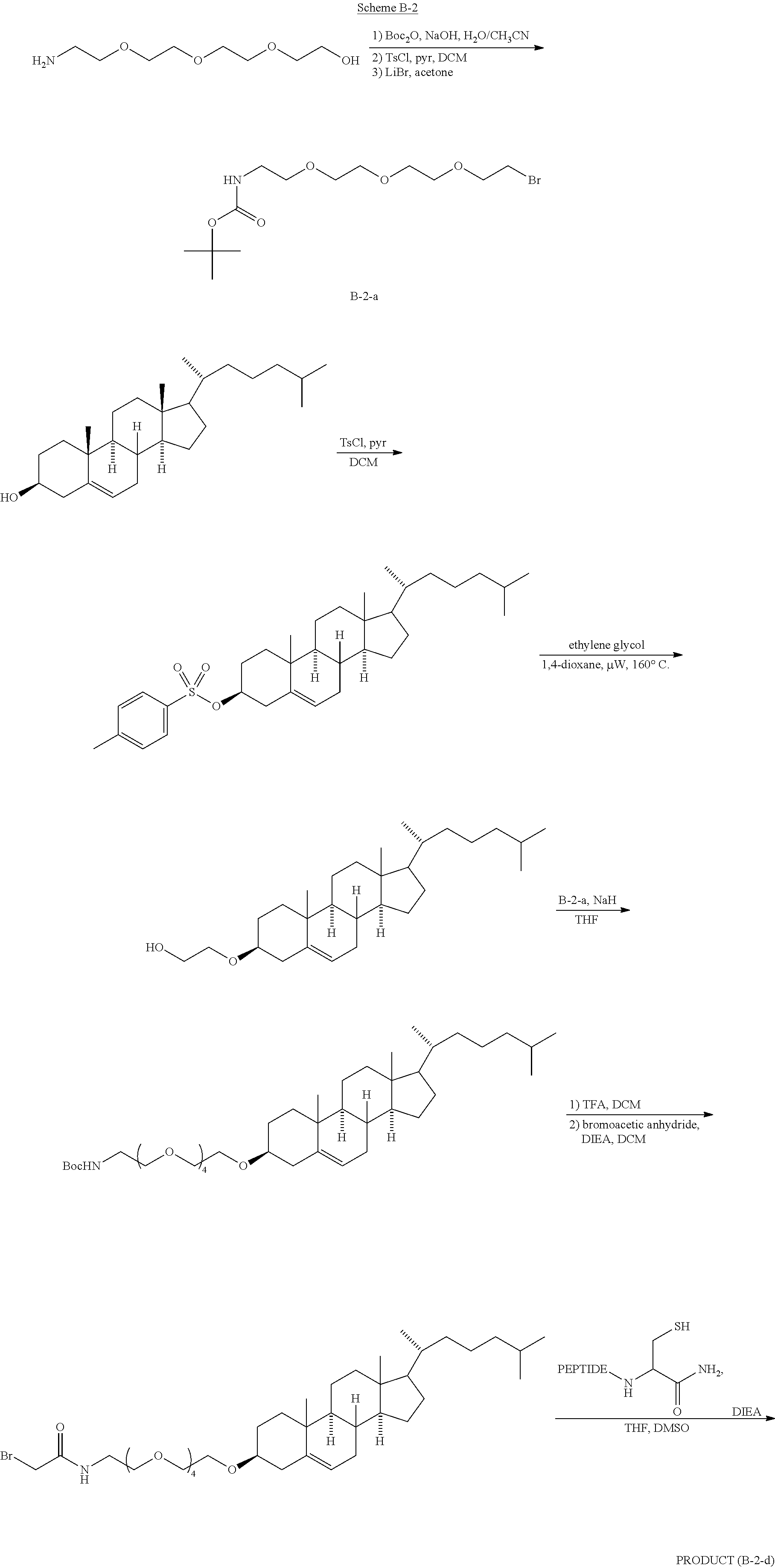

### tert-Butyl 2-(2-(2-(2-bromoethoxy)ethoxy)ethoxy)ethylcarbamate (B-2-a)

A solution of 2-(2-(2-(2-aminoethoxy)ethoxy)ethoxy)ethanol (400 mg, 2.07 mmol, 0.33 mL) in 15 mL of acetonitrile/water (6:1) was treated with di-tert-butyl dicarbonate (603 mg, 2.77 mmol), followed by 2.8 mL of 1 N NaOH (aq). After stirring at RT for 45 min, the organic solvent was removed in vacuo, the residue was dissolved in saturated NH₄Cl (aq), and the desired carbamate was extracted with EtOAc. Removal of EtOAc provided the crude carbamate as a colorless oil.

This oil was dissolved in DCM and treated with p-toluenesulfonyl chloride (1.18 g, 6.21 mmol) and pyridine (0.84 mL, 10.4 mmol). After stirring at 40° C. for 12 h, the mixture was diluted with DCM and washed with 1N HCl (2×10 mL), H₂O (10 mL), and brine (10 mL), then dried over Na₂SO₄ and concentrated. Purification via flash column chromatography on silica gel gave 330 mg (36% yield over 2 steps) of the tosylate as a colorless oil.

A solution of the tosylate (203 mg, 0.453 mmol) in 3.1 mL of anhydrous acetone was treated with LiBr (385 mg, 4.53 mmol). After stirring at 60° C. for 8 h, the solvent was removed and the resulting residue was dissolved in EtOAc. The organic mixture was washed with water, dried over MgSO₄, filtered, and concentrated. Purification via flash column chromatography on silica gel gave 126 mg (78% yield) of the title compound as a colorless oil.

### 2-((3S,8S,9S,10R,13R,14S,17R)-10,13-Dimethyl-17-(6-methylheptan-2-yl)-2,3,4,7,8,9,10,11,12,13,14,15,16,17-tetradecahydro-1H-cyclopenta[a]phenanthren-3-yloxy)ethanol (B-2-b)

A solution of cholesterol (1.50 g, 3.89 mmol) in 4 mL of DCM was treated with p-toluenesulfonyl chloride (1.49 g, 7.78 mmol), pyridine (4 mL), and DMAP (94.9 mg, 0.780 mmol). After stirring at RT for 12 h, the mixture was diluted with DCM and washed with 1N HCl (2×5 mL), H₂O (5 mL), and brine (5 mL), then dried over Na₂SO₄ and concentrated in vacuo. Recrystallization from chloroform and methanol gave 1.66 g (79% yield) of the tosylate intermediate as a white solid.

A microwave vial charged with the tosylate intermediate (500 mg, 0.926 mmol) in 7.7 mL of 1,4-dioxane was treated with 2.6 mL of ethylene glycol. After heating to 160° C. by microwave irradiation for 10 min, the solvent was removed, and the residue was dissolved in chloroform and washed with saturated NaHCO₃ (5 mL), H₂O (5 mL), and brine (5 mL), then dried over Na₂SO₄ and concentrated in vacuo. Purification of the crude material by flash column chromatography using silica gel provided 270 mg (68% yield) of the title compound as a white solid.

### 2-Bromo-N-(14-((3S,8S,9S,10R,13R,14S,17R)-10,13-dimethyl-17-(6-methylheptan-2-yl)-2,3,4,7,8,9,10,11,12,13,14,15,16,17-tetradecahydro-1H-cyclopenta[a]phenanthren-3-yloxy)-3,6,9,12-tetraoxatetradecyl)acetamide (B-2-c)

A solution of B-2-b (219 mg, 0.509 mmol) in 2.5 mL of THF was treated with sodium hydride (60% dispersion in mineral oil, 20.4 mg, 0.509 mmol) and stirred for 30 min. The mixture was cooled to 0° C. and treated with B-2-a (139 mg, 0.392 mmol) in 2.5 mL of THF. After heating to 40° C. for 6 h, the reaction was quenched with saturated NH₄Cl (aq) and extracted with EtOAc. Purification of the crude material by flash column chromatography using silica gel provided 101 mg (36% yield) of the ether intermediate as a colorless oil.

A solution of the ether intermediate (74 mg, 0.105 mmol) in 2.1 mL of DCM was treated with 0.53 mL of TFA. After stirring at RT for 40 min, the mixture was concentrated in vacuo and dissolved in 10 mL DCM. A 4-mL aliquot of this solution was taken into a separate vial, concentrated to a 1-mL volume, cooled to 0° C., and charged with bromoacetic anhydride (13.6 mg, 0.053 mmol) and DIPEA (15 μL, 0.088 mmol). After stirring at RT for 12 h, the reaction mixture was directly purified by flash column chromatography using silica gel to give 12.6 mg (41% over 2 steps) of the title compound as an colorless oil.

Lipid conjugate B-2-d is prepared in an analogous manner as lipid conjugate B-1-a of Example 4, Scheme B-1.

### Example 5. Preparation of Br-FA₁

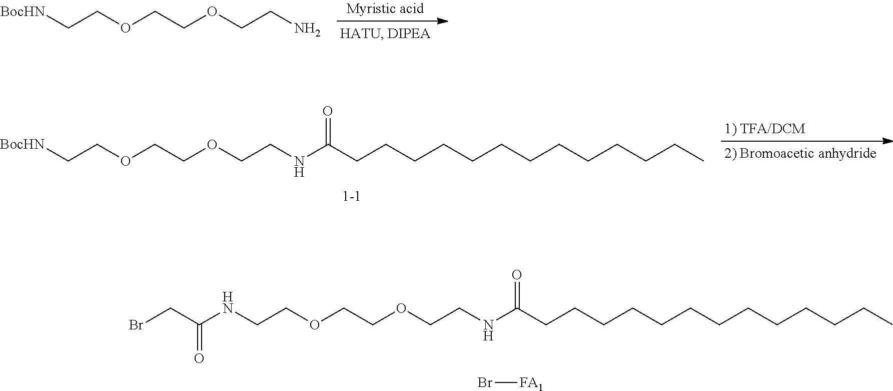

### Step A. Tert-butyl (2-(2-(2-tetradecanamidoethoxy)ethoxy)ethyl)carbamate (1-1)

Myristic acid (0.46 g, 2 mmol) was dissolved in 5 mL of DMF. HATU (0.8 g, 2.1 mmol) and DIPEA (0.4 mL, 2.2 mmol) were added followed by the addition of Boc-NH-PEG2-COOH (0.5 g, 2 mmol). The reaction mixture was then stirred for 6 h, and the solvent was removed. The product was extracted with EtOAc (3×15 mL). The organic layer was successively washed with sat. NaHCO₃, cooled HCl (1 M) and brine, dried over Na₂SO₄, filtered, and concentrated. Purification by flash column chromatography on silica gel provided 0.81 g of desired compound as white solids in 90% product yield. ESI-MS: calcd MW 458.4; found 459.6 [M+1]⁺.

### Step B. N-(2-(2-(2-(2-bromoacetamido)ethoxy)ethoxy)ethyl)tetradecanamide (Br-FA₁)

A solution of 1-1 (0.23 g, 0.5 mmol) in DCM (10 mL) was treated with TFA (2 mL) for 2 h. The mixture was concentrated and followed by the addition of bromoacetic anhydride (0.14 g, 0.55 mmol), DIPEA (0.17 mL, 1 mmol) in 10 mL of DCM at 0° C. The reaction mixture was then stirred for 2 h, and the solvent was removed. The product was extracted with EtOAc (3×15 mL). The organic layer was successively washed with sat. NaHCO₃, cooled HCl (1 M) and brine, dried over Na₂SO₄, filtered, and concentrated. Purification by flash column chromatography on silica gel provided 0.2 g of the title compound as white solids in 83% product yield. ESI-MS: calcd MW 479.5; found 480.4 [M+1]⁺.

### Example 6. Solid-Phase Synthesis of Br-FA₂

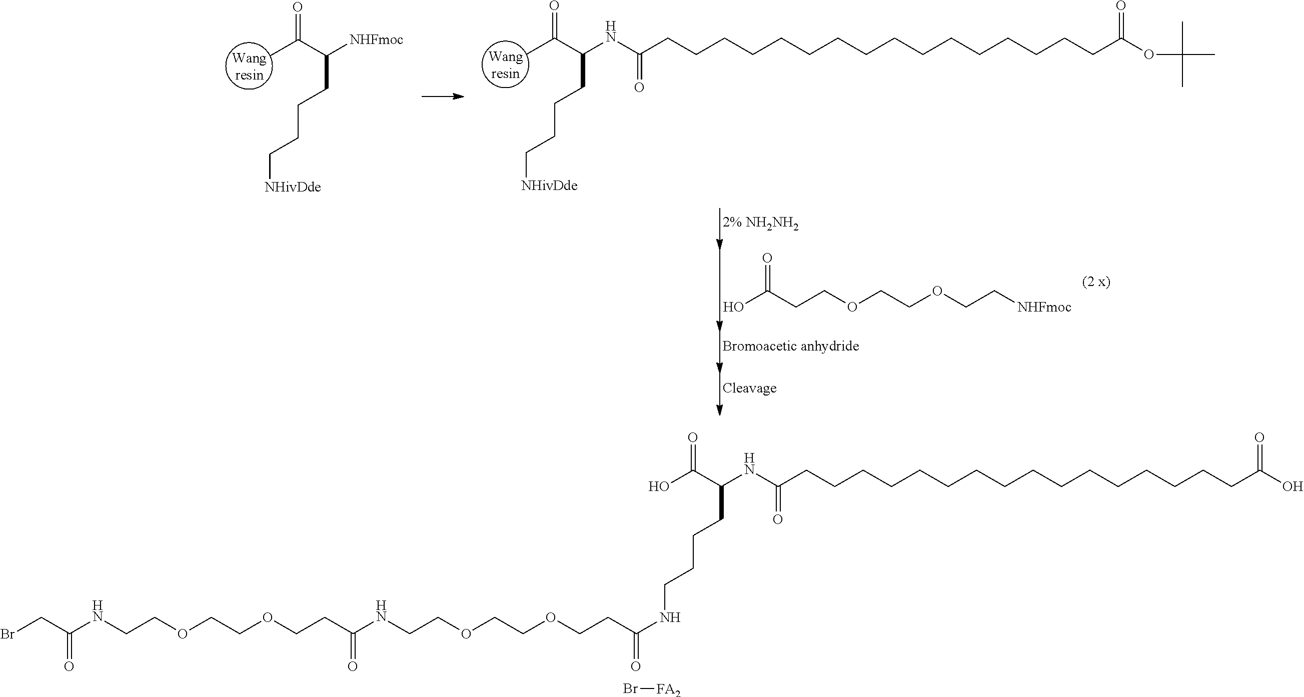

Fmoc-Lys(ivDde)-OH (60 mg, 100 μmol) was attached to 2-chlorotrityl chloride resin (Novabiochem) (100 mg, 80 μmol) by mixing the amino acid, the resin, and DIPEA (70 μL, 400 μmop in 5 mL of DMF and stirring for 30 min. The resin was then washed with DMF (3×), DCM (3×) and treated with CH₃OH/DCM/DIPEA (8:1:1) for 10 min to cap the unreacted trityl chloride sites, dried under vacuum, and stored in a desiccator.

To this resin (50 mg, 40 μmol) was added piperidine in DMF (20%, 5 mL). The mixture was shaken for 1 min and drained. Another 5 mL of 20% piperidine was added and this time the mixture was shaken for 15 min. Positive ninhydrin test was observed. The resin was then washed as described above.

The resin was then treated with octadecanedioic acid mono-tert-butyl ester (AstaTech) (74 mg, 200 μmol) using coupling reagent HATU (76 mg, 200 μmol), and DIPEA (35 μL, 200 μmop in DMF (5 mL) for 2 h or repeated until a negative ninhydrin test was observed. After washing with DMF and DCM, the resin was treated with 2% hydrazine in DMF (5 mL, 2×5 min). Positive ninhydrin test was observed. The resin was then washed as described above.

The resin was then treated with Fmoc-PEG2-propionic acid (Quanta BioDesign) (80 mg, 200 μmop using HATU (76 mg, 200 μmol), and DIPEA (35 μL, 200 μmol) in DMF (5 mL) for 2 h or repeated until a negative ninhydrin test was observed. The resin was then washed as described above. Then the protecting group is removed and the above steps are repeated.

The resin was then treated with bromoacetic anhydride (55 mg, 200 μmol), and DIPEA (35 μL, 200 μmol) in 2 mL of DCM and stirring for 30 min. After washing with DCM (3×), the product was cleaved from the resin using 5 mL of 10% TFA in DCM containing 10% H₂O and 10% triisopropylsilane for 1 h. After cleavage, TFA was removed under reduced pressure. The resulting yellow residue was washed several times with cold diethyl ether and was finally dried to a crude product as yellow powder under nitrogen flow.

Attempts to dissolve 50 mg of the crude product in up to 0.5 mL of 50% CH₃CN in water (0.1% TFA) have been unsuccessful. Therefore, as an alternative, the crude peptide, (50 mg) was dissolved in DMSO (0.1 mL) and this solution was diluted to a final volume of 0.5 mL with 50% CH₃CN-water. The solution was filtered. The filtered solution was loaded onto the preparative HPLC column (Phenomenex, Prep C18, 300A, 50×250 mm) equilibrated with 10% CH₃CN (0.1% TFA) in water (0.1% TFA), and the column was eluted with 10% CH₃CN (0.1% TFA) in water (0.1% TFA) to wash DMSO from the column. The composition of the eluent then was ramped to 35% CH₃CN-water (0.1% TFA) over 1 min, and a linear gradient was initiated at a rate of 0.5%/min of CH₃CN (0.1% TFA) into water (0.1% TFA) and run for 50 min. Eluted fractions were checked for purity on an analytical reversed phase C18 column (Phenomenex, C18, 120A, 4.6×50 mm) and fractions containing the product in >95% purity were combined and freeze-dried to afford the title compound (15 mg, 41% yield). The molecular weight of product was analyzed by ESI-MS: calcd MW 880.9; found 881.3 [M+1]⁺, 882.6 [M+2]⁺.

### Example 7. Solid-Phase Synthesis of Br-FA₃

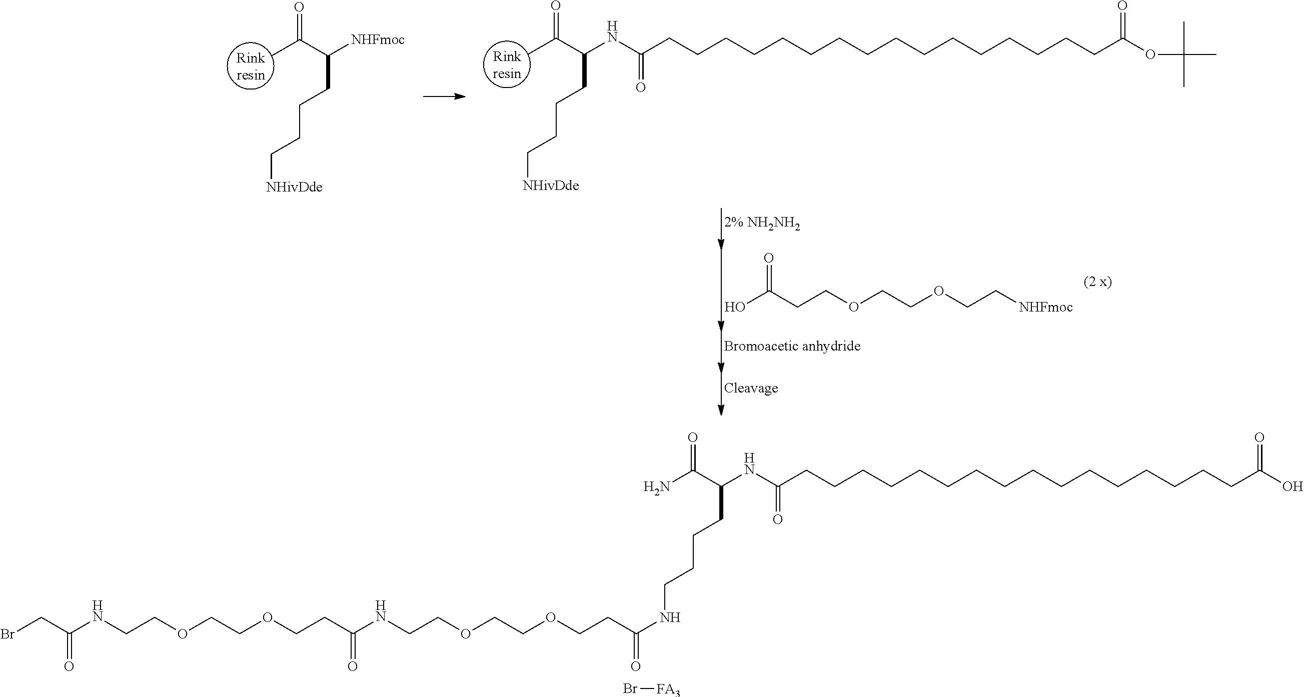

The title compound was prepared in an analogous manner as Br-FA₂ by substituting 2-chlorotrityl chloride resin with rink amide resin. Purification by preparative HPLC and lyophilization gave the title compound as a white powder with greater than 95% purity in about 45% product yield. The molecular weight of peptide was analyzed by ESI-MS: calcd MW 881.0; found 882.2 [M+1]⁺, 883.7 [M+2]⁺.

### Example 8. Preparation of Br-FA₄

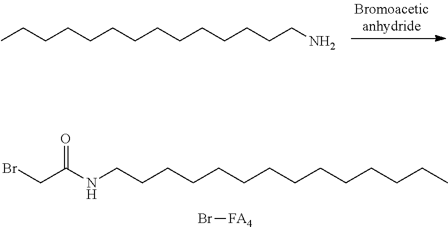

A solution of tetradecylamine (107 mg, 0.5 mmol) in DCM (10 mL) was treated with bromoacetic anhydride (0.14 g, 0.55 mmol), DIPEA (100 μL, 0.55 mmol) at 0° C. The reaction mixture was then stirred for 2 h, and the solvent was removed. The product was extracted with EtOAc (3×15 mL). The organic layer was successively washed with sat. NaHCO₃, cooled HCl (1 M) and brine, dried over Na₂SO₄, filtered, and concentrated. Purification by flash column chromatography on silica gel provided 145 mg of the title compound as white solids in 87% product yield. ESI-MS: calcd MW 334.3; found 335.6 [M+1]⁺.

### Example 9. Preparation of Br-FA₅

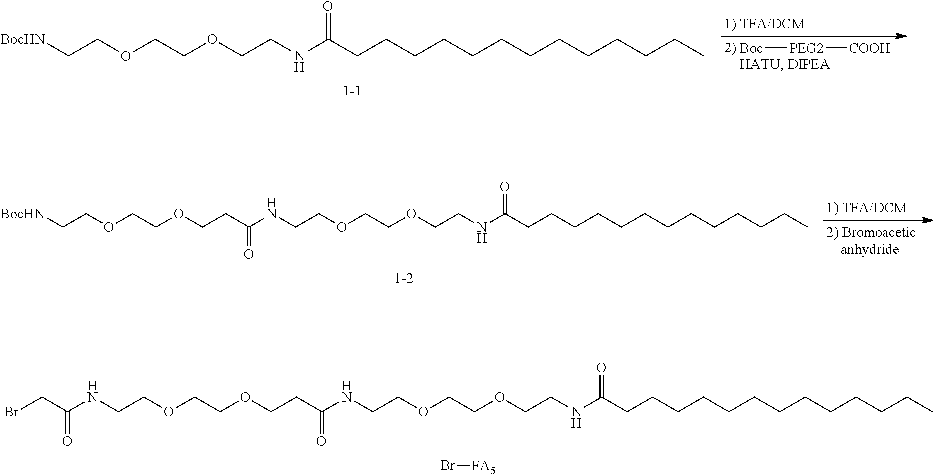

### Step A. Tert-butyl (9,20-dioxo-3,6,13,16-tetraoxa-10,19-diazatritriacontyl)carbamate (1-2)

A solution of 1-1 (115 mg, 0.25 mmol) in DCM (10 mL) was treated with TFA (2 mL) for 2 h. The mixture was concentrated and followed by the addition of Boc-NH-PEG2-acid (70 mg, 0.25 mmol), HATU (95 mg, 0.25 mmol) and DIPEA (90 μL, 0.5 mmol) in 5 mL of DMF. The reaction mixture was then stirred for 2 h, and the solvent was removed. The product was extracted with EtOAc (3×15 mL). The organic layer was successively washed with sat. NaHCO₃, cooled HCl (1 M) and brine, dried over Na₂SO₄, filtered, and concentrated. Purification by flash column chromatography on silica gel provided 122 mg of desired compound as white solids in 77% product yield. ESI-MS: calcd MW 617.9; found 618.4 [M+1]⁺.

### Step B. N-(1-bromo-2,12-dioxo-6,9,16,19-tetraoxa-3,13-diazahenicosan-21-yl)tetradecanamide (Br-FA₅)

A solution of 1-2 (122 mg, 0.2 mmol) in DCM (10 mL) was treated with TFA (2 mL) for 2 h. The mixture was concentrated and followed by the addition of bromoacetic anhydride (52 mg, 0.2 mmol), DIPEA (70 μL, 0.4 mmol) in 5 mL of DCM at 0° C. The reaction mixture was then stirred for 2 h, and the solvent was removed. The product was extracted with EtOAc (3×15 mL). The organic layer was successively washed with sat. NaHCO₃, cooled HCl (1 M) and brine, dried over Na₂SO₄, filtered, and concentrated. Purification by HPLC provided 88 mg of the title compound as white solids in 69% product yield. ESI-MS: calcd MW 638.7; found 639.4 [M+1]⁺.

### Example 10. Preparation of Br-FA₆

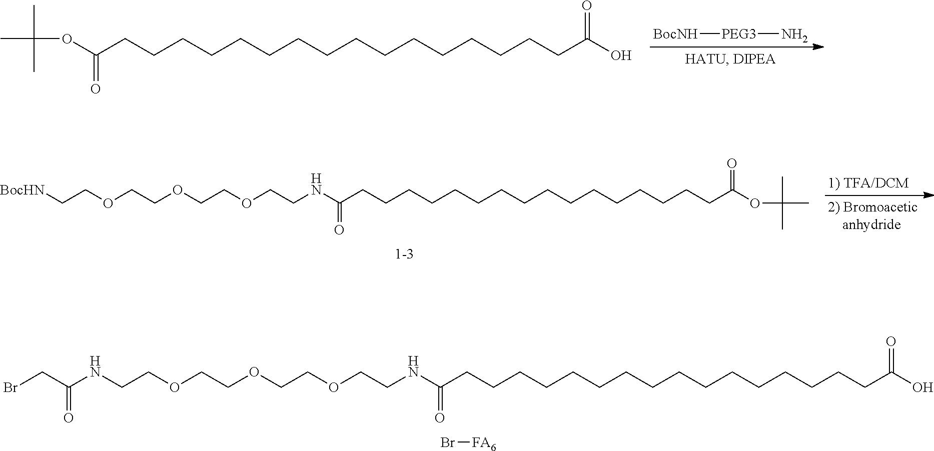

### Step A. tert-Butyl (2-(2-(2-tetradecanamidoethoxy)ethoxy)ethyl)carbamate (1-3)

Octadecanedioic acid mono-tert-butyl ester acid (0.18 g, 0.5 mmol) was dissolved in 5 mL of DMF. HATU (0.2 g, 0.55 mmol) and DIPEA (0.1 mL, 0.55 mmol) were added followed by the addition of Boc-NH-PEG3-NH₂ (0.12 g, 0.5 mmol). The reaction mixture was then stirred for 6 h, and the solvent was removed. The product was extracted with EtOAc (3×10 mL). The organic layer was successively washed with sat. NaHCO₃, cooled HCl (1 M) and brine, dried over Na₂SO₄, filtered, and concentrated. Purification by flash column chromatography on silica gel provided 0.28 g of tert-butyl (2-(2-(2-tetradecanamidoethoxy)ethoxy)ethyl)carbamate as white solids in 87% product yield. ESI-MS: calcd MW 644.5; found 645.5 [M+1]⁺.

### Step B. 1-Bromo-2,16-dioxo-6,9,12-trioxa-3,15-diazatritriacontan-33-oic acid (Br-FA₆)

A solution of 1-3 (65 mg, 0.1 mmol) in DCM (2 mL) was treated with TFA (4 mL) for 4 h. The mixture was concentrated and followed by the addition of bromoacetic anhydride (26 mg, 0.1 mmol), DIPEA (35 μL, 0.2 mmol) in 5 mL of DCM at 0° C. The reaction mixture was then stirred for 2 h, and the solvent was removed. The product was purified by HPLC provided 35 mg of the title compound as white solids in 70% product yield. ESI-MS: calcd MW 608.3; found 609.4 [M+1]⁺.

### Example 11. Solid Phase Synthesis of Br-FA₇

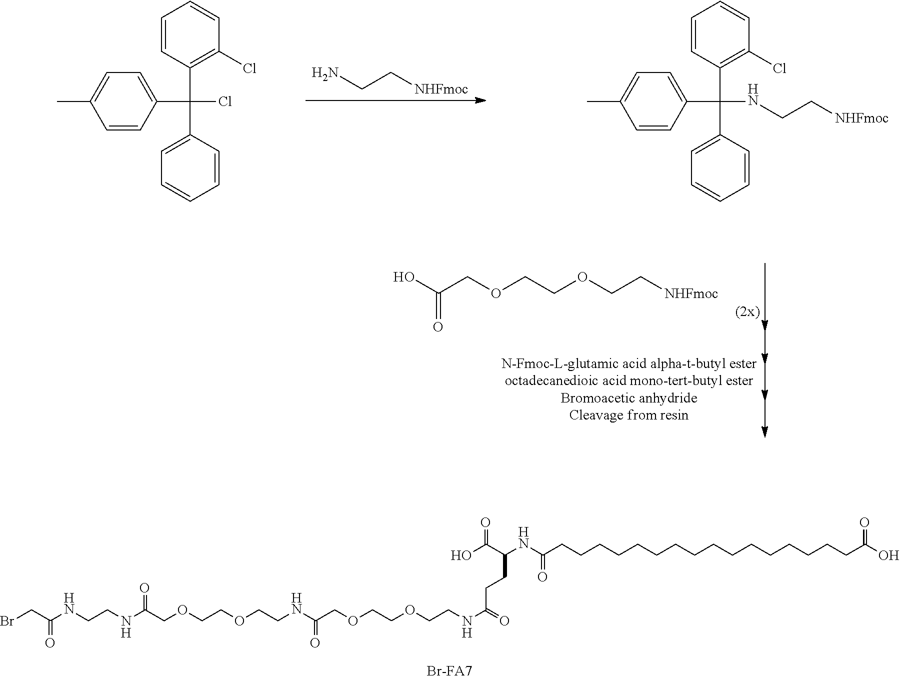

The title compound was prepared in an analogous manner as Br-FA₂ by substituting Fmoc-Lys(ivDde)-OH and Fmoc-PEG2-propionic acid with mono-Fmoc ethylene diamine hydrochloride and [2-[2-(Fmoc-amino)ethoxy]ethoxy]acetic acid, respectively. Purification by preparative HPLC and lyophilization afforded the title compound as a white powder with greater than 95% purity in about 25% product yield. The molecular weight of peptide was analyzed by ESI-MS: calcd MW 896.9; found 898.1 [M+1]⁺.

### Example 12. Preparation of Br-FA₈

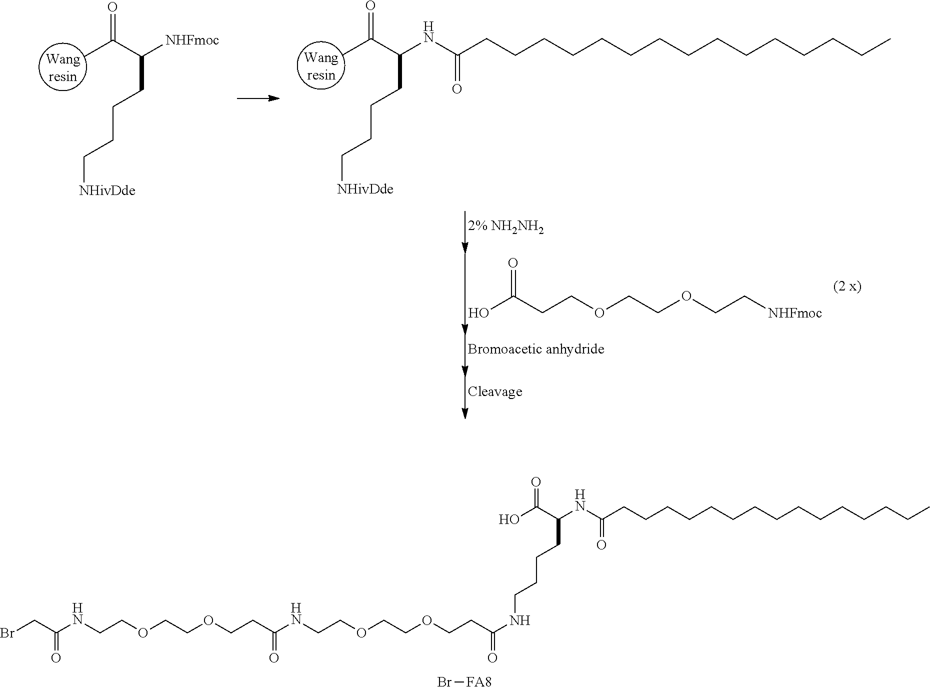

The title compound was prepared in an analogous manner as Br-FA₂ by substituting octadecanedioic acid mono-tert-butyl ester with palmitic acid. Purification by preparative HPLC and lyophilization afforded the title compound as a white powder with greater than 95% purity in about 29% product yield. The molecular weight of peptide was analyzed by ESI-MS: calcd MW 822.4; found 823.6 [M+1]⁺.

### Example 13. Preparation of Br-FA155

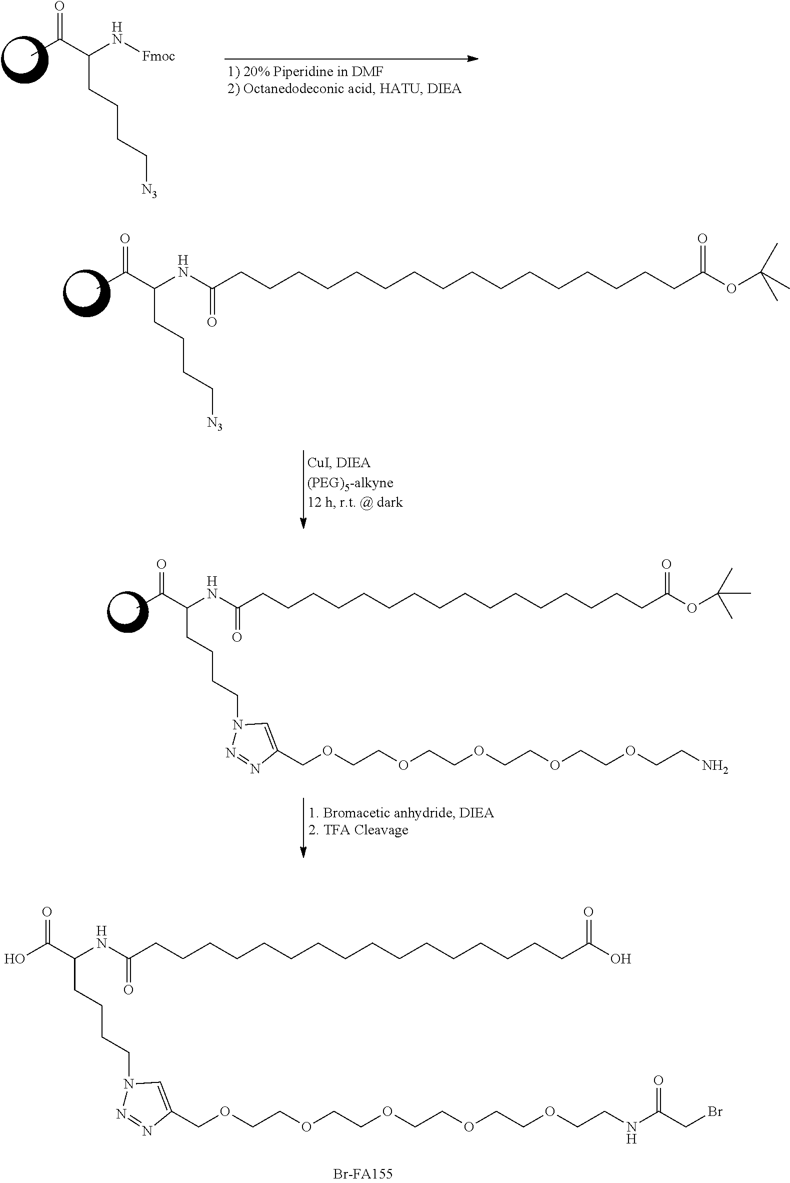

Fmoc-Lys(N₃)—OH (40 mg, 100 μmop was added to wang resin (100 mg, 70 μmol) along with HATU (40 mg), DIPEA (200 μL) in 2 mL of DMF and stirred for 30 min. The resin was then washed with DMF (3×), DCM (3×) and then treated with acetic anhydride/DIPEA for 10 min to cap the unreacted wang resin. The resin was dried under vacuum, and stored in a desiccator. The Fmoc group was subsequently cleaved using 20% piperidine in DMF for 20 minutes, and this process was repeated three times. After checking for the completion of cleavage using ninhydrin, the resin was washed with DMF and DCM.

The deprotected resin was then treated with octadecanedioic acid mono-tert-butyl ester (AstaTech) (74 mg, 200 μmol), HATU (76 mg, 200 μmol), and DIPEA (200 μL) in DMF (5 mL) for 2 h. The coupling was repeated until ninhydrin test was negative. After washing with DMF and DCM, the resin was treated with 10% copper iodide, (PEG)₅-alkyne (40 mg), and DIPEA (0.2 mL) in DMF (1 mL). The resin was then treated with bromoacetic anhydride (100 mg, 200 μmop, and DIPEA (200 μL) in 2 mL of DCM and stirred for 2 h. After washing with DCM (3×), the product was cleaved from the resin using 5 mL of 50% TFA in DCM containing 10% H₂O and 10% triisopropylsilane for 2 h. After cleavage, the cleaved solution was dried and then re-dissolved by methanol/DMF before subjecting to HPLC purification to afford Br-FA155 (3 mg, 15% yield). ESI-MS calculated MW 864.5; found 866 [M+1]⁺.

### Example 14. Preparation of Br-FA175

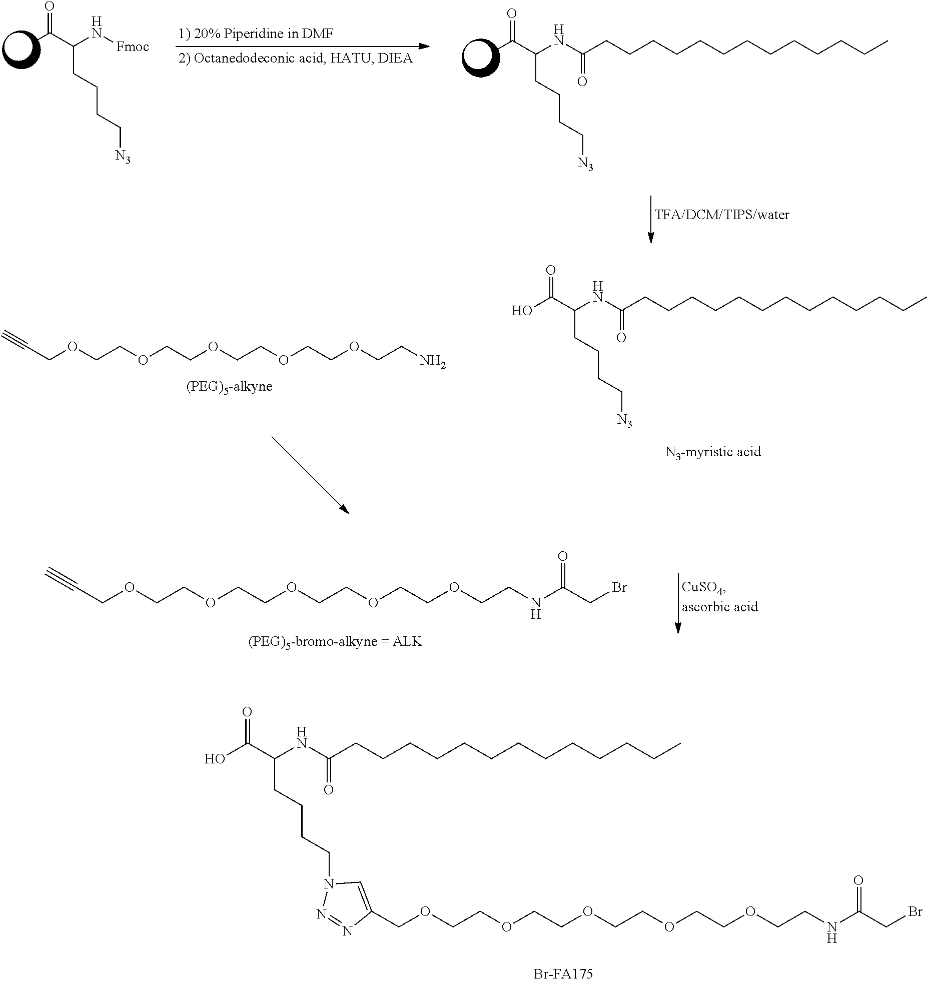

Fmoc-Lys(N₃)—OH (40 mg, 100 μmop was added to wang resin (100 mg, 70 μmol) along with HATU (40 mg), DIPEA (200 μL) in 2 mL of DMF and stirred for 30 min. The resin was then washed with DMF (3×), DCM (3×) and then treated with acetic anhydride/DIPEA for 10 min to cap the unreacted wang resin. The resin was dried under vacuum, and stored in a desiccator. The Fmoc group was subsequently cleaved using 20% piperidine in DMF for 20 minutes, and this process was repeated three times. After checking for the completion of cleavage using ninhydrin, the resin was washed with DMF and DCM. The deprotected resin was then treated with myristic acid (100 mg), HATU (180 mg), and DIPEA (200 μL) in DMF (2 mL) for 2 h. The resin was cleaved to generate N₃-myristic acid. ESI-MS calculated molecular weight 382.5; found 383.5 [M+1]+.

(PEG)₅-bromo-alkyne (ALK) was prepared by addition of bromoacetic anhydride (40 mg) to DIPEA (100 μL), DCM (1 mL), (PEG)₅-alkyne (20 mg). The product was purified by HPLC and lyophilized to provide the desired product (15 mg, 70% yield). ESI-MS calculated molecular weight 395.5; found 396.5 [M+1]⁺.

N₃-myristic acid (0.5 mg) was added to (PEG)₅-bromo-alkyne (0.75 mg) along with copper sulfate (2 mg) and ascorbic acid (2 mg) in 1:1 acetonitrile and water and the pH was adjusted to 8 and stirred overnight to provide Br-FA175 (0.7 mg, 70% yield). ESI-MS calculated 779.5, found 780.5 [M+1]⁺.

### Example 15. Preparation of Br-FA182

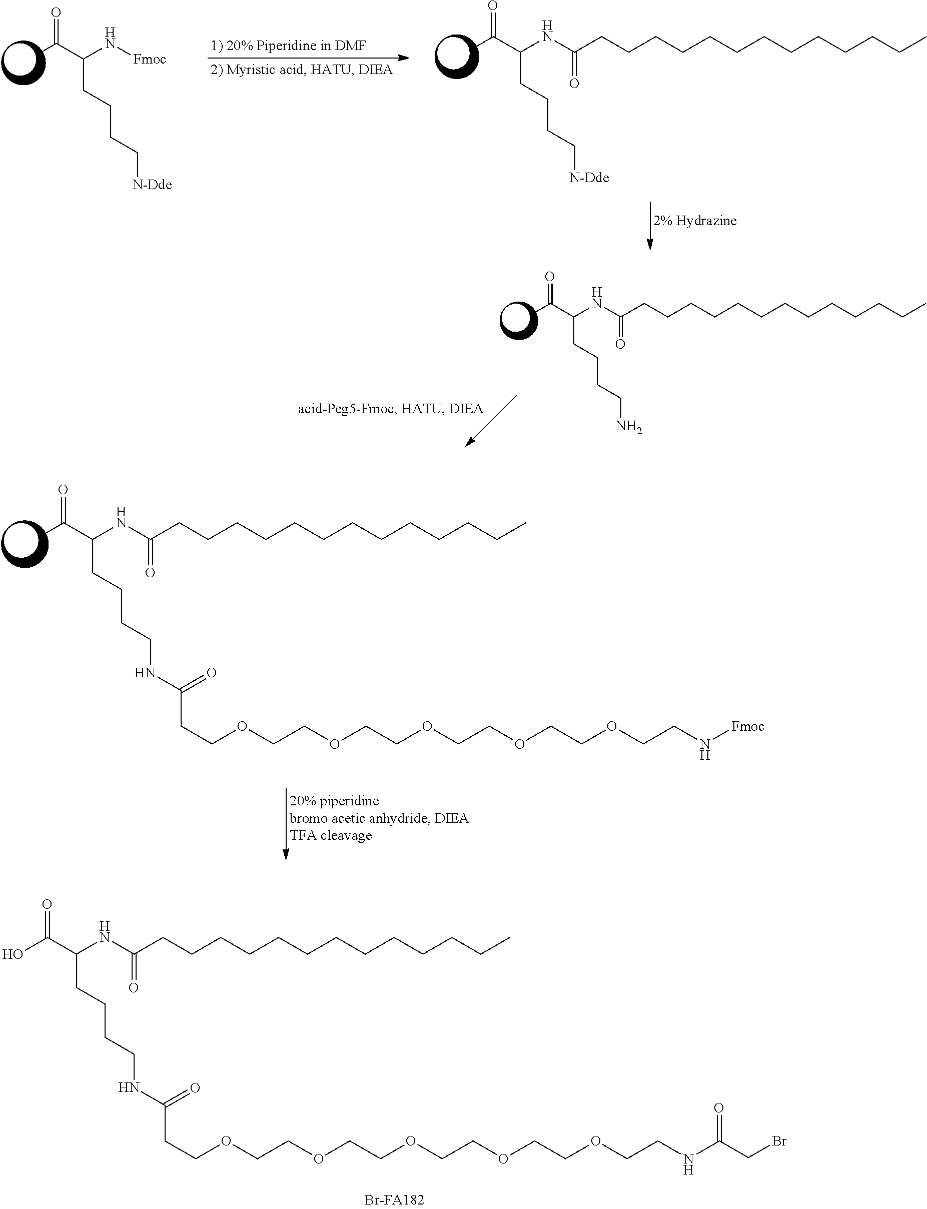

Fmoc-Lys-ε-N-Dde 500 mg of resin (0.15 mmoles) was treated with 20% piperidine and then washed with DMF (3×), DCM (3×). The resin was then treated with Myristic acid (10 eq.) along with DIEA, HATU and the coupling continued for 2 hours. Dde was removed using 2% Hydrazine in DMF (2×) and then washed with DMF and DCM. Acid-Peg5-Fmoc acid was then coupled using DIEA, HATU overnight. After confirming the coupling reaction with nihydrin test, Fmoc was cleaved and then the free amine is allowed to react with bromo acetic anhydride in the presence of base to generate the desired compounds FA-182 on resin. After washing with DCM (3×), the product was cleaved from the resin using 5 mL of 50% TFA in DCM containing 10% H₂O and 10% triisopropylsilane for 2 h. After cleavage, the cleaved solution was died and then re-dissolved by methanol/DMF before subjecting to HPLC purification. The fractions sonjtaing the product were combined and freese dried to afford FA-182 (ESI-MS calculated MW 768.2; found 769.4 [M+1]⁺.

### Example 16. Preparation of Br-FA183

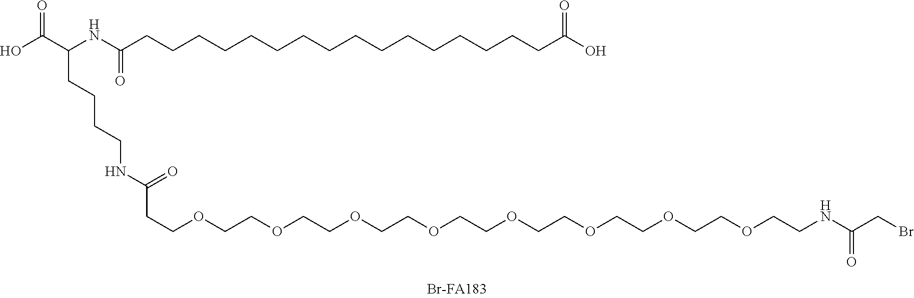 [SMILES: O=C(O)CCCCCCCCCCCCCCCCC(=O)NC(CCCCNC(=O)CCOCCOCCOCCOCCOCCOCCOCCOCCNC(=O)CBr)C(=O)O]

The title compound was prepared in an analogous manner as Br-FA182 by substituting myristic acid with octadecanedioic acid mono-tert-butyl ester. Purification by preparative HPLC and lyophilization afforded the title compound as a white powder. The molecular weight of peptide was analyzed by ESI-MS: calcd MW 987.2; found 988.1 [M+1]⁺.

### Example 17. Preparation of Br-FA186

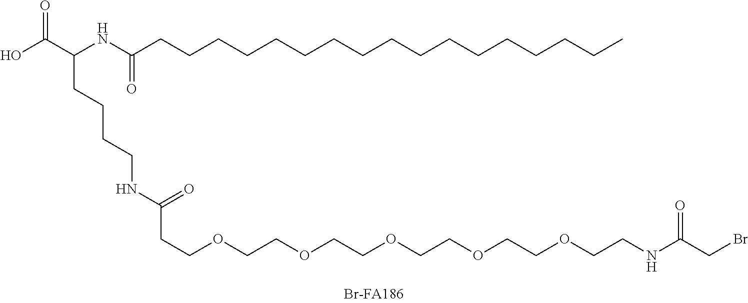 [SMILES: CCCCCCCCCCCCCCCCCC(=O)NC(CCCCNC(=O)CCOCCOCCOCCOCCOCCNC(=O)CBr)C(=O)O]

The title compound was prepared in an analogous manner as Br-FA182 by substituting myristic acid with stearic acid. Purification by preparative HPLC and lyophilization afforded the title compound as a white powder. The molecular weight of peptide was analyzed by ESI-MS: calcd MW 823.4; found 824.3 [M+1]⁺.

### Example 18. Preparation of Inclusion Bodies

Cells expressing relaxin analogs were spun down to produce a cell pellet. The cell pellet was re-suspended in lysis buffer (100 mM Tris, pH 8, 100 mM NaCl, 1% Triton-X100, 2 mM EDTA) and lysed by passing through French pressure cell operated at 18,000 psi for at least 3 times while chilling the cell suspension to 4° C. after each pass. Lysed cell suspension was clarified by centrifuging for 45 min at 15,000 rpm, 4° C. The pellet contains intact cells, cellular debris along with inclusion body protein. The pellet was washed multiple times to isolate the inclusion body protein from the rest, briefly, after decanting the supernatant, the pellet was re-suspended in lysis buffer using a tissue homogenizer and subjected to centrifugation, this procedure was repeated at least thrice. To remove the Triton-X 100 from the pellet, the pellet was washed at least twice with wash buffer (100 mM Tris, pH 8, 100 mM NaCl, 1% Triton-X100, 2 mM EDTA); washing of pellet was performed by resuspension through homogenization followed by centrifugation. The pellet was then directly used for in vitro refolding of the relaxin analog without any further purification.

### Example 19. Refolding of Relaxin Analogs

The cell pellet from Example 18 was used for in vitro refolding of relaxin analogs. Refolding of the relaxin analogs was carried using oxidized/reduced glutathione. Inclusion bodies were dissolved in 8M guanidine hydrochloride (GdnHCl). 40 mL of GdnHCl was used to dissolve inclusion body pellet isolated from 1 L E. coli culture. After solubilizing the protein, the insoluble portion was pelleted by centrifuging at 15000 rpm for 30 min. The solubilized protein was then concentrated using 3,000 MW cutoff Amicon Ultra (Fisher Scientific) centrifugal filter unit to 10-15 mL. The denatured protein was then diluted in to 300 mL of refolding buffer (440 mM Arginine, 55 mM Tris, pH 8.2 containing 2 mM GSH, 10 mM GSSG). The refolding was continued for 1-2 hours and quenched by adding TFA to pH 2. The precipitate was then centrifuged to a pellet and the supernatant was transferred in to centrifugal unit and concentrated to 2-3 mg/mL. The concentrated protein mixture was then purified by semi-preparative HPLC using Phenomenex Jupiter C5 column (250×4.6 mm) with the flow rate set at 4.0 mL/min. The fractions were analyzed by LC-MS for the desired protein and then pooled and lyophilized.

### Example 20. Conjugation of Lipid to Relaxin

All the enzymatic processing (Trypsin/Cp-B or TEV protease) were performed after capping cysteine, as the free thiol in relaxin was observed to cause scrambling at basic pH where enzymatic digestion are generally performed. The lyophilized protein from Example 19 was dissolved in 0.25% TFA. Pegylated lipid was dissolved in ACN acetonitrile/100 mM NH₄HCO₃ buffer (1:1), pH 8.5. Relaxin was then added to the mixture and the reaction proceeded at room temperature for 2 hours; the progress of the reaction was checked by LC-MS. After completion of reaction, the mixture was then lyophilized.

### Example 21. Enzymatic Conversion of Relaxin Precursor into Two-Chain Precursor

The lyophilized powder from Example 20 was resuspended in 50 mM Tris buffer containing 1 mM CaCl₂, pH8. Trypsin (1:100) was added to this mixture, and the mixture was incubated for 1 h at room temperature. After 1 h, carboxy peptidase-B (10 units for 1 mg of protein) was added in to the mixture and the progress of the cleavage was monitored by LC-MS analysis. After the complete cleavage, the pH of the mixture was brought to 4 by adding acetic acid. The mixture was lyophilized and purified semi-preparative HPLC.

### Example 22. Cleavage of His-Tag Through TEV Protease

The lyophilized protein of Example 21 was re-dissolved in PBS buffer containing 3 mM GSH: 0.5 mM GSSG. Tev protease was added in to the mixture and incubated overnight at room temperature. The cleavage of tag was confirmed by LC-MS analysis.

### Example 23. Expression of the B:D1A-A:Q1-L2C-Relaxin Analog

The inserts were prepared through polymerase cycling assembly, where forward and reverse primers were annealed to generate the DNA fragment which was further enriched by forward and reverse end primers.

The amplified fragment was then digested with restriction digestion enzymes (NdeI/KpnI). The prepared insert was then ligated into a vector with a PET backbone under the control of a T7 promoter and terminator. The encoded DNA fragment was then confirmed by DNA sequencing.

For expression, Relaxin constructs were transformed into K12 E. coli (derivative of W3110) with a genomic T7 RNA polymerase gene under the control of an araBAD promoter.

The transformed E. coli cells were cultured in LB supplemented with 50 μg/ml kanamycin to OD600 nm=0.8 at 37° C. with vigorous shaking (250 rpm).

To induce expression of the genes, L-arabinose stock solution (20% w/v in H₂O) was added to the final concentration of 0.2%, and the cells were continuously incubated with shaking at 250 rpm at 37° C. for 5 h. Cells were then harvested by centrifugation (6000 rpm, 15 min).

### Example 24. Refolding of the B:D1A-A:Q1-L2C-Relaxin Analog

Refolding of the B:D1A, A:Q1-L2C-relaxin analog was carried using oxidized/reduced glutathione. Inclusion bodies were dissolved in 8M guanidine hydrochloride (GdnHCl). 40 mL of GdnHCl was used to dissolve inclusion body (Preparation of inclusion bodies was performed as described in Example 18) as pellet isolated from 1 L E. coli culture. After solubilizing the protein, the insoluble portion was pelleted by centrifuging at 15000 rpm for 30 min. The solubilized protein was then concentrated using 3,000 MW cutoff Amicon Ultra (Fisher Scientific) centrifugal filter unit to 10-15 mL. The denatured protein was then diluted in to 300 mL of refolding buffer (440 mM Arginine, 55 mM Tris, pH 8.2 containing 2 mM GSH, 10 mM GSSG). The refolding was continued for 1-2 hours and quenched by adding TFA to pH 2. The precipitate was then centrifuged to a pellet and the supernatant was transferred in to centrifugal unit and concentrated to 2-3 mg/mL. The concentrated protein mixture was then purified by semi-preparative HPLC using Phenomenex Jupiter C5 column (250×4.6 mm) with the flow rate set at 4.0 mL/min. The fractions were analyzed by LC-MS for the desired protein and then pooled and lyophilized, expected mass: 8167.4, observed mass: 8167.

### Example 25. Conjugation of Br-FA2 to B:D1A-A:Q1-L2C Relaxin Analog

The lyophilized B:D1A-A:Q1-L2C relaxin analog from example 24 was dissolved in 0.25% TFA. Br-FA2 was dissolved in ACN acetonitrile/100 mM NH₄HCO₃ buffer (1:1), pH 8.5. The AL2C-relaxin analog was then added to the mixture and the reaction proceeded at room temperature for 2 hours. The progress of the reaction was checked by LC-MS. After completion of reaction, the mixture was then lyophilized and purified to yield pure the B:D1A-A:Q1-L2C-FA2 relaxin analog, expected mass: 8968.5, observed mass: 8968.94.

### Example 26. Enzymatic Processing of B:D1A-A:Q1-L2C-FA2 Relaxin Analog Precursor into Two-Chain Relaxin

The lyophilized powder from Example 25 was resuspended in 50 mM Tris buffer containing 1 mM CaCl₂, pH 8. Trypsin (1:100) was added to this mixture, and the mixture was incubated for 1 h at room temperature. After 1 h, carboxy peptidase-B (10 units for 1 mg of protein) was added in to the mixture and the progress of the cleavage was monitored by LC-MS analysis. After the complete cleavage, the pH of the mixture was brought to 4 by adding acetic acid. The mixture was lyophilized and purified by semi-preparative HPLC to yield relaxin-A:Q-L2C-FA2, expected mass: 6727.1, observed mass: 6727.3.

### Example 27. Synthesis of A:pQ1-L2C-FA2

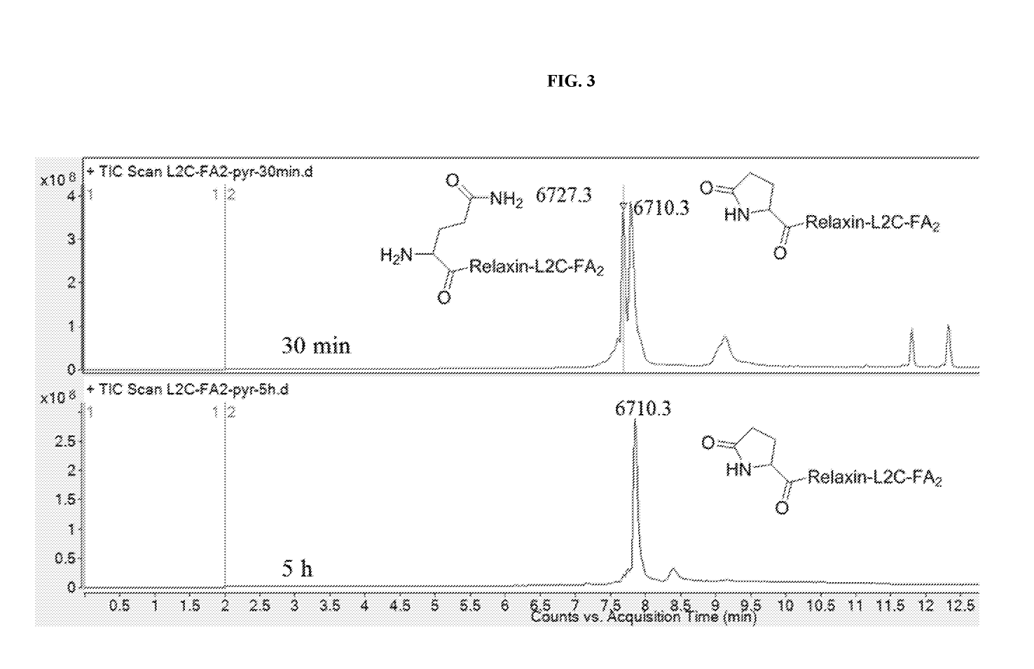

The lyophilized relaxin A:Q1-L2C-FA2 was resuspended in 50 mM acetic acid and then incubated at 90° C. for 6 hours to see complete conversion to the glutamine (Q) to the pyroglutamate (pQ) by Q-TOF analysis, expected mass: 6710.1, observed mass: 6710.3 (See FIG. 3)

### Example 28. Conjugation of FA-ALK, FA155, FA175, FA182, FA183, and FA186 to B:D1A-A:Q1-L2C Relaxin Analog

The conjugation and processing into two-chain relaxin was done as described in Examples 25 and 26 using FA155, FA175, FA182, FA183, and FA186 as the starting materials. The observed Masses are listed in Table 1:

TABLE 1
observed masses:

| Name | Observed Mass |
|---|---|
| Relaxin B:D1A-A:Q1-L2C-FA155 | 8172.5 |
| Relaxin B:D1A-A:Q1-L2C-FA175 | 8086.5 |
| Relaxin B:D1A-A:Q1-L2C-FA182 | 6614.2 |
| Relaxin B:D1A-A:Q1-L2C-FA183 | 6832.4 |
| Relaxin B:D1A-A:Q1-L2C-FA186 | 6670.1 |

### Example 29. Gene Construction and Expression of Toxin-550 Analogs

The inserts were prepared through polymerase cycling assembly, where forward and reverse primers were annealed to generate the DNA fragment which was further enriched by forward and reverse primers. The amplified fragment was then digested with restriction digestion enzymes (Bam I/Hind-III). The prepared insert was then ligated into a vector with a PET backbone, the encoded DNA fragment was then confirmed by DNA sequencing. Toxin-550 constructs (pET/Txin-550) were transformed into E. coli strain BL21 (DE3). The transformed E. coli cells were cultured in terrific broth (with 50 μg/ml kanamycin) to OD600 nm=1.0 at 37° C. with vigorous shaking (250 rpm). To induce expression of the proteins, IPTG stock solution (1M) was added to the final concentration of 0.5 mM, and the cells were continuously incubated with shaking at 250 rpm at 26° C. for 24 h. On the following day, cells were harvested by centrifugation (6000 rpm, 15 min). Preparation of inclusion bodies was performed as described in Example 18.

### Example 30. Refolding of Toxin-550 Analogs

Refolding was carried using oxidized/reduced glutathione. Inclusion bodies (as in Example 18) were dissolved in 8M guanidine hydrochloride (GdnHCl). 10 mL of GdnHCl was used to dissolve the inclusion body pellet isolated from 1 L E. coli culture. After solubilizing the protein, another 10 mL of Tris-HCL buffer (50 mM) was added and the insoluble portion was pelleted by centrifuging at 15000 rpm for 30 min. The solubilized supernatant denatured protein was then diluted in to 200 mL of refolding buffer. Various refolding buffers that were used to dilute the solubilized supernatant denatured protein are shown in the table below:

| Refolding Buffer | GSH:GSSG |
|---|---|
| 0.1M Tris, pH 8.2 | 1:10 |
| 0.1M Tris, pH 11 | 1:10 |
| 0.1M NH₄HCO₃, pH 8 | 1:10 |
| 0.1M NH₄OAC | 1:10 |
| 2M NH₄OAC | 1:10 |
| 2M (NH₄)₂SO₄ | 1:10 |

FIG. 5 shows the disulfide bond pattern for the Toxin-550 peptide (wild-type) and an exemplary in vitro folding pathway of cysteine-knot Toxin-550 peptide, the middle structure depicting a two-sulfide bond intermediate, in which a disulfide bond is formed between cysteine I and cysteine IV and between cysteine II and cysteine V.

Various refolding trails were employed, with most of the refolding buffer conditions leading to a two-disulfide bond intermediate as major product, with the exception of 2 M (NH₄)₂SO₄ refolding buffer, where the three disulfide bond product is observed in 4 hours. The three disulfide bond product contains a Toxin-550 peptide with a disulfide bond formed between cysteine I and cysteine IV, between cysteine II and cysteine V, and between cysteine III and cysteine VI.

### Example 31. Conjugation of Lipid to Toxin-550

The refolded toxin-550 was then buffer exchanged with PBS, 2% TFA to remove the excess glutathione before conjugation with lipids. Pegylated lipid was dissolved in ACN acetonitrile/100 mM NH₄HCO₃ buffer (1:1), pH 8.5. Toxin was then added to the mixture and the reaction proceeded at room temperature for 2 h; the progress of the reaction was checked by LC-MS. After completion of reaction, the mixture was then lyophilized and purified by RP-HPLC using C18-phenomenex column.

### Example 32. Conjugation of Lipid to Oxyntomodulin and Exenatide Derivatives

The oxyntomodulin Cys38 (e.g., Oxm-Cys38, SEQ ID NO: 24) and exenatide Cys40 (e.g., Ex4-Cys40, SEQ ID NO: 25) were chemically synthesized using solid-phase peptide synthesis. Pegylated lipid was dissolved in ACN acetonitrile/100 mM NH₄HCO₃ buffer (1:1), pH 8.5. The cysteine mutant was dissolved in ACN acetonitrile/100 mM NH₄HCO₃ buffer and then added to the pegylated lipid mixture and the reaction proceeded at room temperature for 2 hours. Progress of the reaction was checked by LC-MS. After completion of reaction the mixture was then lyophilized and purified by RP-HPLC using C18-phenomenex column.

The following compounds in Table 2 were or are prepared using analogous procedures.

TABLE 2

| entry | Structure* |
|---|---|
| 1 | 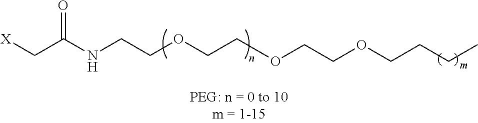 |
| 2 | 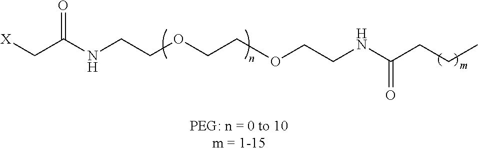 |
| 3 | 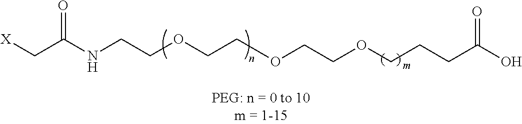 |
| 4 | 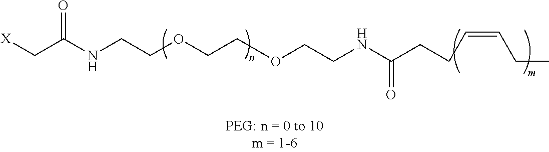 |
| 5 | 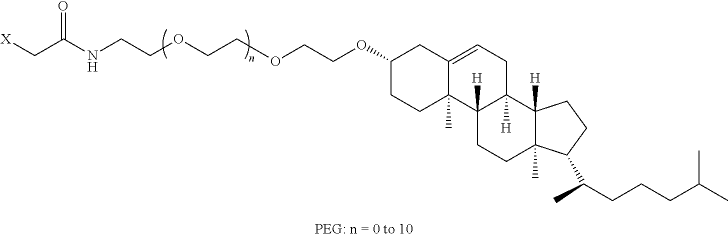 |
| 6 | 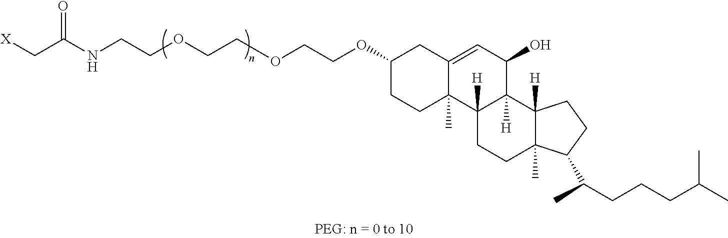 |
| 7 | 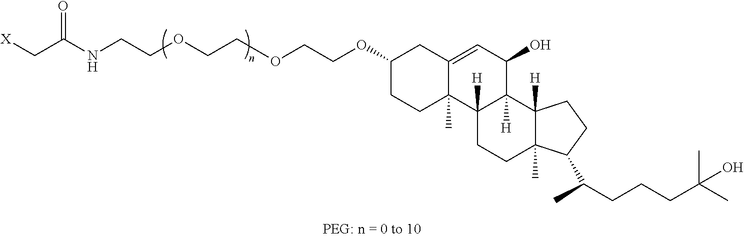 |
| 8 | 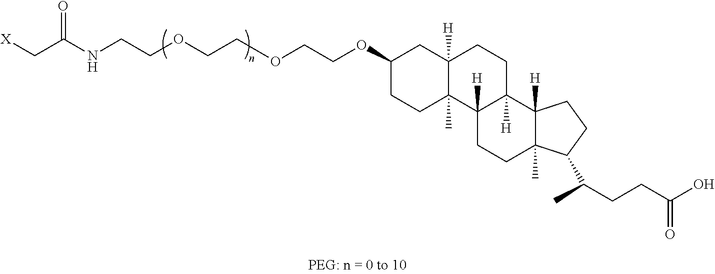 |
| 9 | 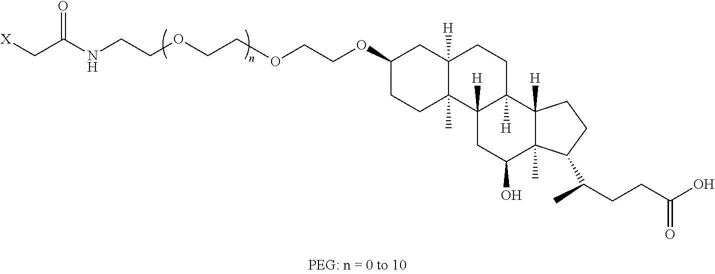 |
| 10 | 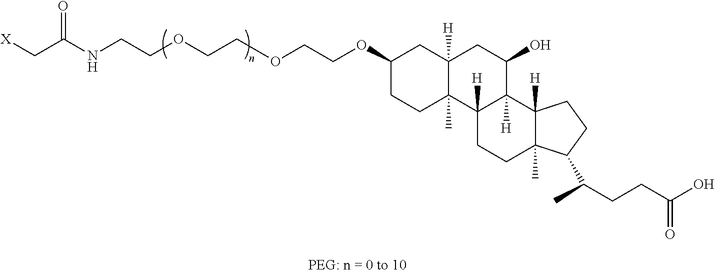 |
| 11 | 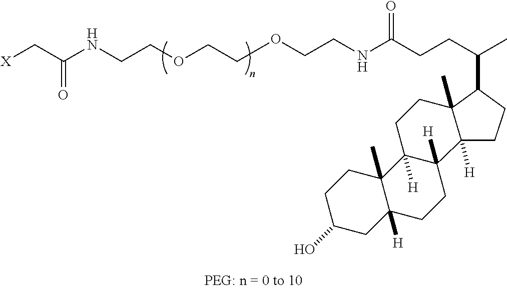 |
| 12 | 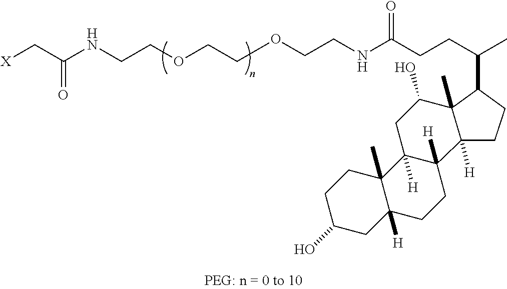 |
| 13 | 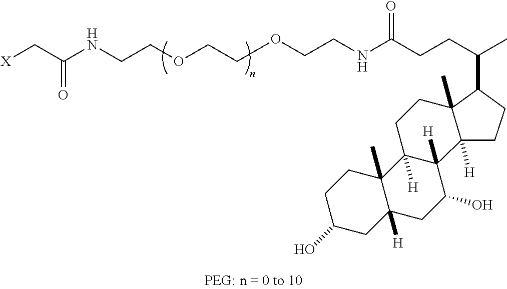 |
| 14 | 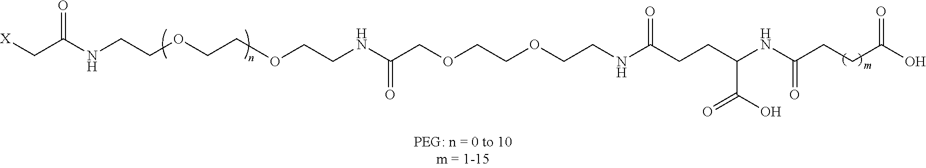 |
| 15 | 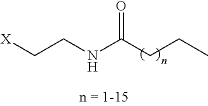 |
| 16 | 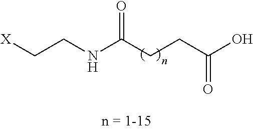 |
| 17 | 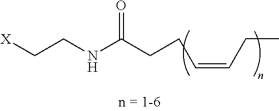 |
| 18 | 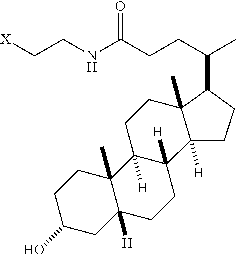 |
| 19 | 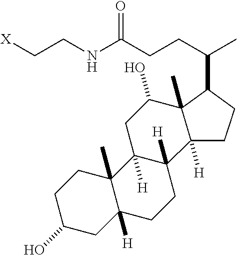 |
| 20 | 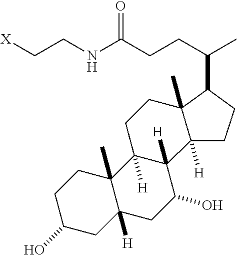 |
| 21 | 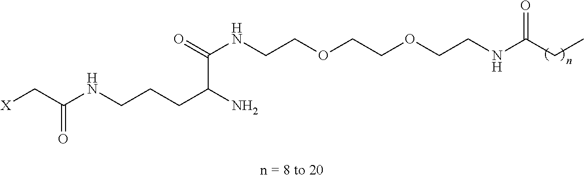 |
| 22 | 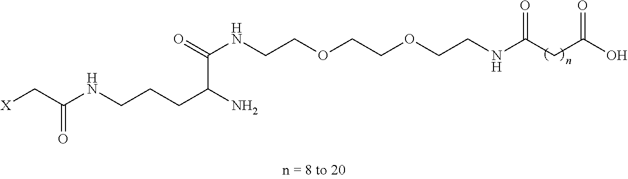 |
| 23 | 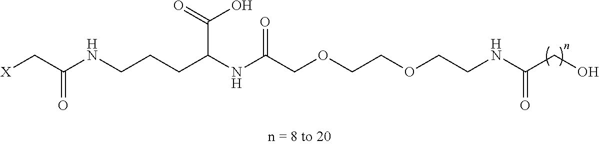 |
| 24 | 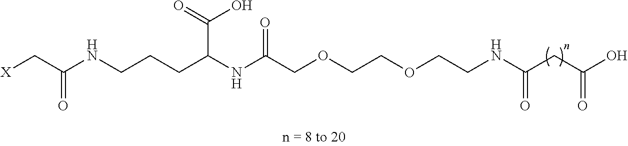 |
| 25 | 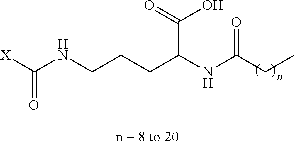 |
| 26 | 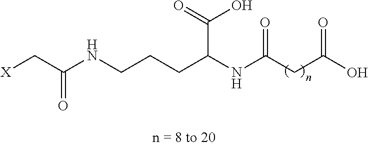 |
| 27 | 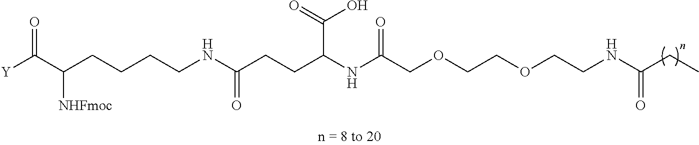 |
| 28 | 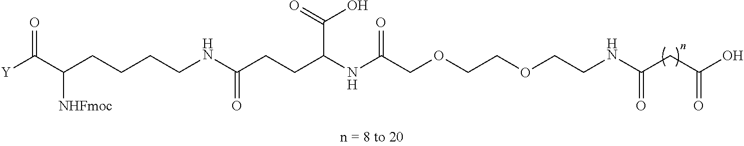 |
| 29 | 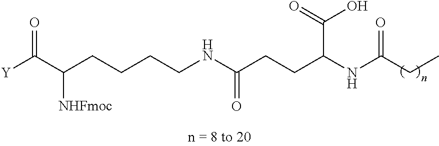 |
| 30 | 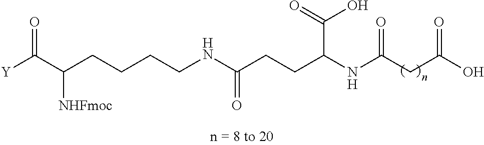 |
| 31 | 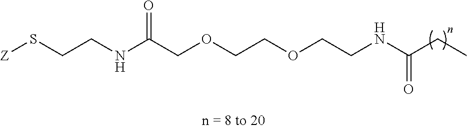 |
| 32 | 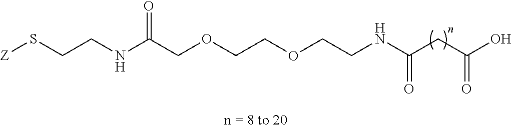 |
| 33 | 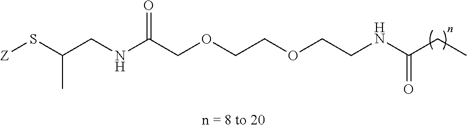 |
| 34 | 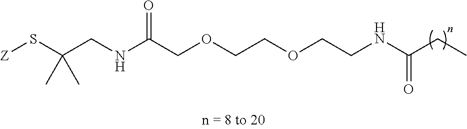 |
| 35 | 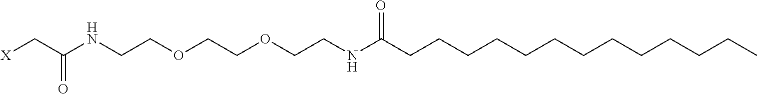 |
| 36 | 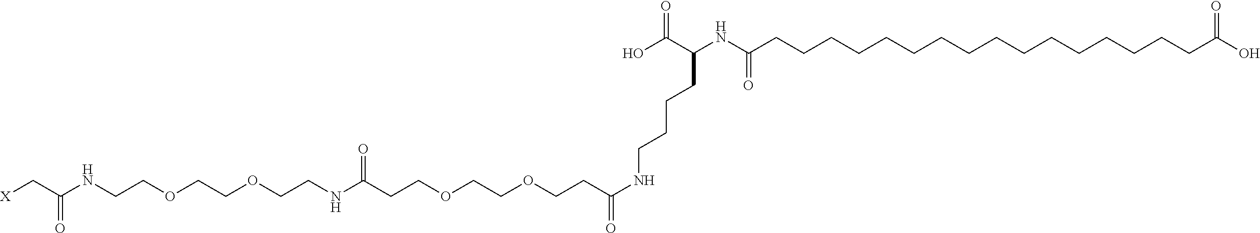 |
| 37 | 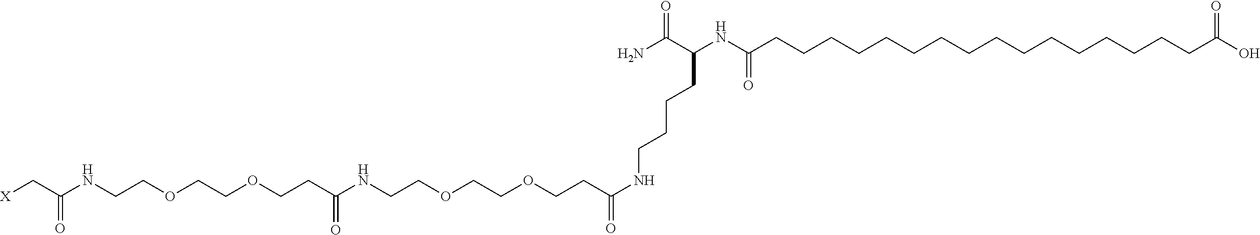 |
| 38 | 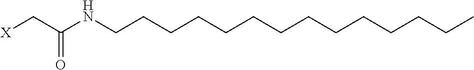 |
| 39 | 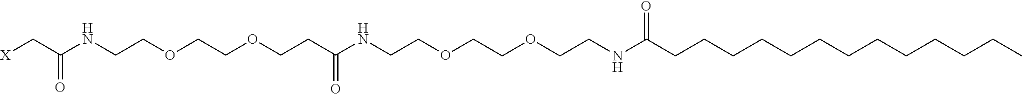 |
| 40 | 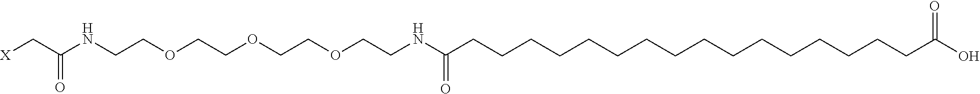 |
| 41 | 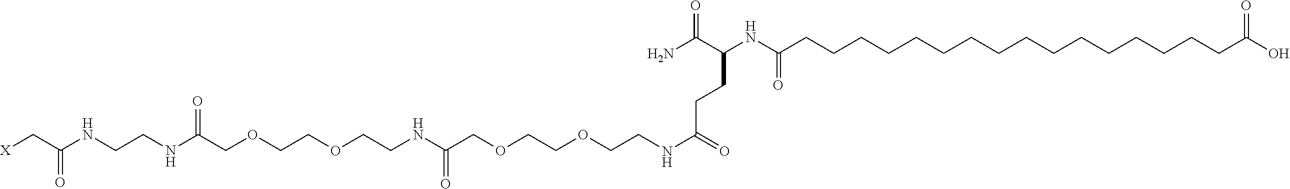 |
| 42 | 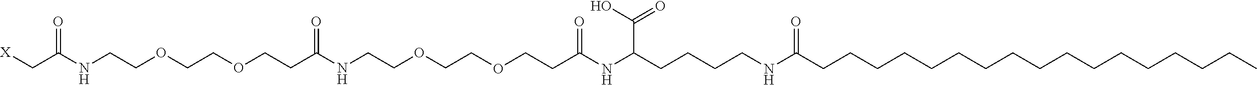 |

*X can be Cl, I, Br, maleimide, or an amino acid which is part of the peptide (TA);
Y can be OH or an amino acid which is part of the peptide (TA);
Z can be S which is part of an amino acid which is part of the peptide (TA) or
S which is a part of a lipid derivative (to form a disulfide).

### Example 33. Measuring Bioactivity of Ex4 and Oxm Peptides

Ex4 is a GLP1R agonist, and Oxm is a GLP1R and GCGR dual agonist. In vitro activity is determined based on the stimulation of adenylate cyclase activity in Hek293 cells stably expressing the GLP1R and GCGR receptor. Stable GCGR and GLP1R receptor expressing cells are maintained in Dulbecco's modified Eagle's medium/F-12 media supplemented with 200 ng/ml Zeocin (Invitrogen). A cyclic AMP responsive element driven luciferase (CRE-Luc) reporter line is generated by lentiviral transduction of these receptor overexpressing HEK293T cells with CRE-Luc lentivirus (Qiagen) and selected with puromycine (2 μg/mL) for two weeks. A reporter gene assay is used to detect cAMP levels and GLP1R and GCGR activation by Ex-4 or Oxm.

Assay detail: HEK293T cells expressing GLP1R and CRE-Luc are seeded at a density of 5×10³ cells per well in 50 μL of Dulbecco's modified Eagle's medium/F-12 medium containing 10% FBS in 384-well solid bottom white plates. Cells are pre-incubated at 37° C. for overnight. Different concentrations of peptides (from 0.001 nM to 100 nM) are added in triplicate, incubated for 18 hours and luciferase activity is detected by adding 104, of Bright Glo (Promega). Luminescence is recorded on Envision (Perkin Elmer). EC₅₀ is calculated after non-linear curve fitting. For GCGR activity, a reporter cell line with GCGR and CRE-Luc is employed.

### Example 34. Measuring Bioactivity of Toxin Peptides

Toxin-550 peptides are assayed on the IonFlux HT using standard running conditions for measurement of NaV 1.7 current. Briefly, HEK293 cells overexpressing the alpha and beta-1 subunits of human Nav1.7 (PrecisION, Millipore) are trypsinized, brought up in serum-free media and allowed to shake at room temperature for 30 minutes. Compounds are diluted into standard extracellular Ringer's solution (ECS) at desired concentration. Cells are washed once with ECS and then brought up in ECS at a concentration of 4 million cells/mL. IonFlux HT plate is set up according to standard protocols.

NaV 1.7 current is measured on the IonFlux according to the following voltage step protocol:

1. The cells are held at −90 mV with a period of 10 seconds.

2. The cells are stepped from −90 mV to −120 mV for 100 ms.
a. then to −10 mV for 30 ms and
b. then back to −90 mV for 30 ms.

Baseline current is allowed to stabilize for 5 minutes, then a control baseline (ECS only) is established. Compound is applied for 20 minutes, and then a positive control (peptide NaV1.7 blocker) is applied.

Leak subtraction is then performed on the data and the resulting currents are analyzed as follows:
1. Cursor subtraction (baseline-peak) is performed for all current sweeps to determine the amplitude of current (I).
2. ECS baseline current for each patch is taken as maximum current (Iₘₐₓ).
3. For a given time (e.g., 10 minutes post-compound addition), percent block is calculated as I/Iₘₐₓ.

### Example 35. In Vivo Pharmacokinetic (PK) Study for Half-Life Extension

Mouse PK:

In vivo i.v. and s.c. PK study were carried out in CD1 female mice (n=5) with relaxin or relaxin FA conjugates (0.3 mg/kg in NH₄Ac buffer).To ensure accurate determination of active relaxins, plasma samples collected at intervals between 0-24 hrs were analyzed using the cell-based reporter gene assay.

Bioanalysis: Serum samples were diluted (1:100) in 5 point serial dilutions (1:3) and measured RXFP1 luciferase reporter activity following assay protocol. Injected peptide were used as a standard to get standard curve and obtained parameters for Bottom, Top, EC₅₀, Hill Slope. Obtained activity values (RLU) were selected (in the linear range of the standard curve) were used to calculate the concentrations in plasma (nmol/L) using equation from standard curve (Y=Bottom+(Top-Bottom)/(1+10̂((Log IC50−X)*Hill Slope))

Rat PK:

In vivo i.v. and s.c. PK study were carried out in Dahl salt sensitive (DSS) female rats (n=4) with relaxin B:D1A-A:pGLu1-L2C-FA2 (0.3 mg/kg in NH₄Ac buffer). To ensure accurate determination of active relaxins, plasma samples collected at intervals between 0-24 hrs were analyzed using the cell-based reporter gene assay.

Bioanalysis: Serum samples were diluted (1:100) in 5 point serial dilutions (1:3) and measured RXFP1 luciferase reporter activity following assay protocol. Injected peptide were used as a standard to get standard curve and obtained parameters for Bottom, Top, EC₅₀, Hill Slope. Obtained activity values (RLU) were selected (in the linear range of the standard curve) were used to calculate the concentrations in plasma (nmol/L) using equation from standard curve (Y=Bottom+(Top-Bottom)/(1+10̂((Log IC50−X)*Hill Slope))

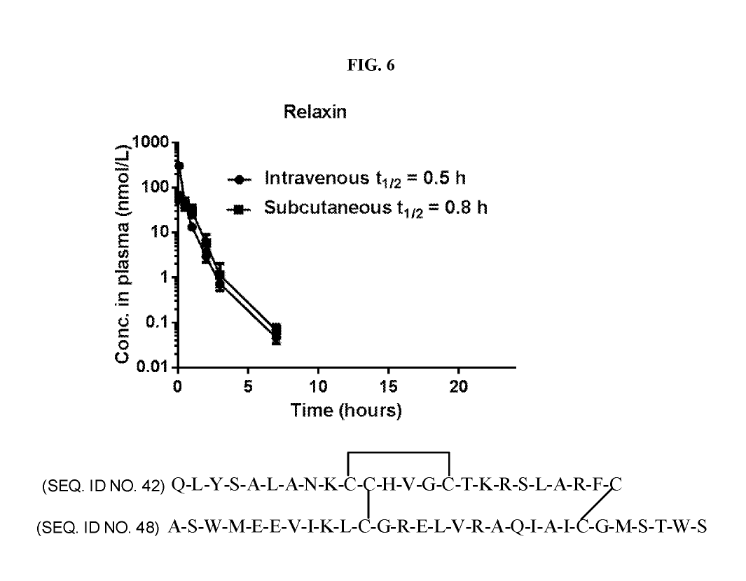

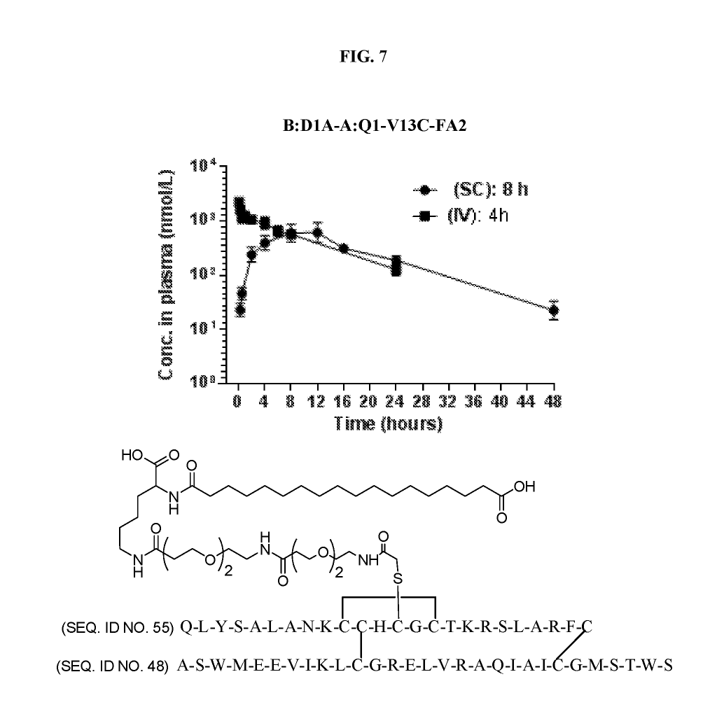

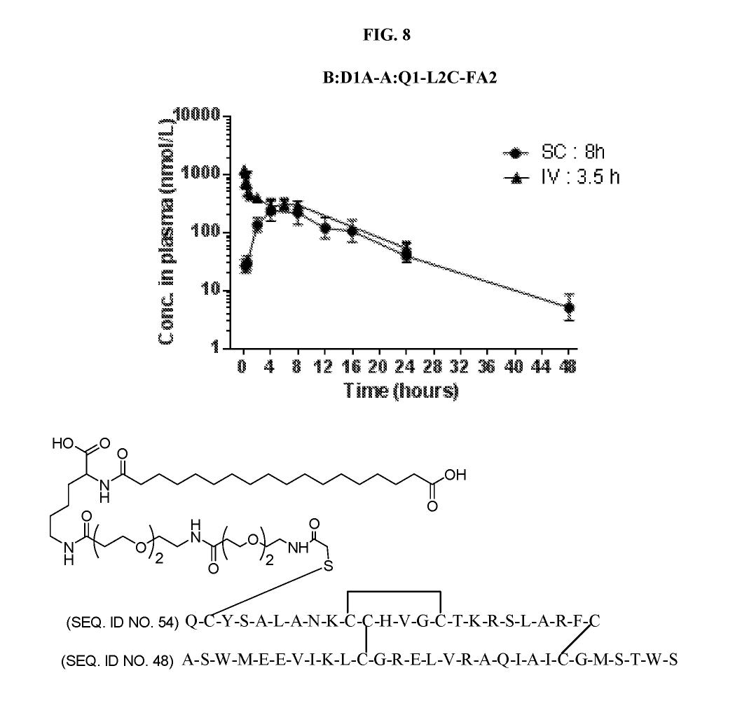

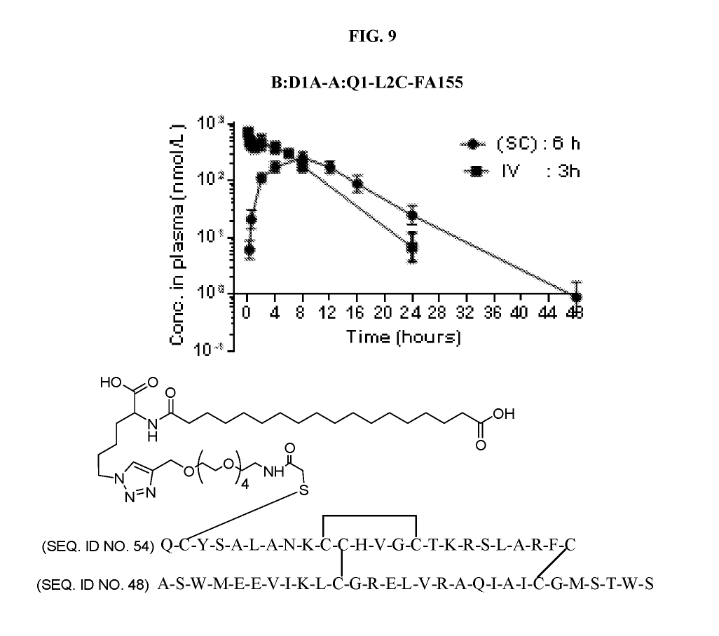

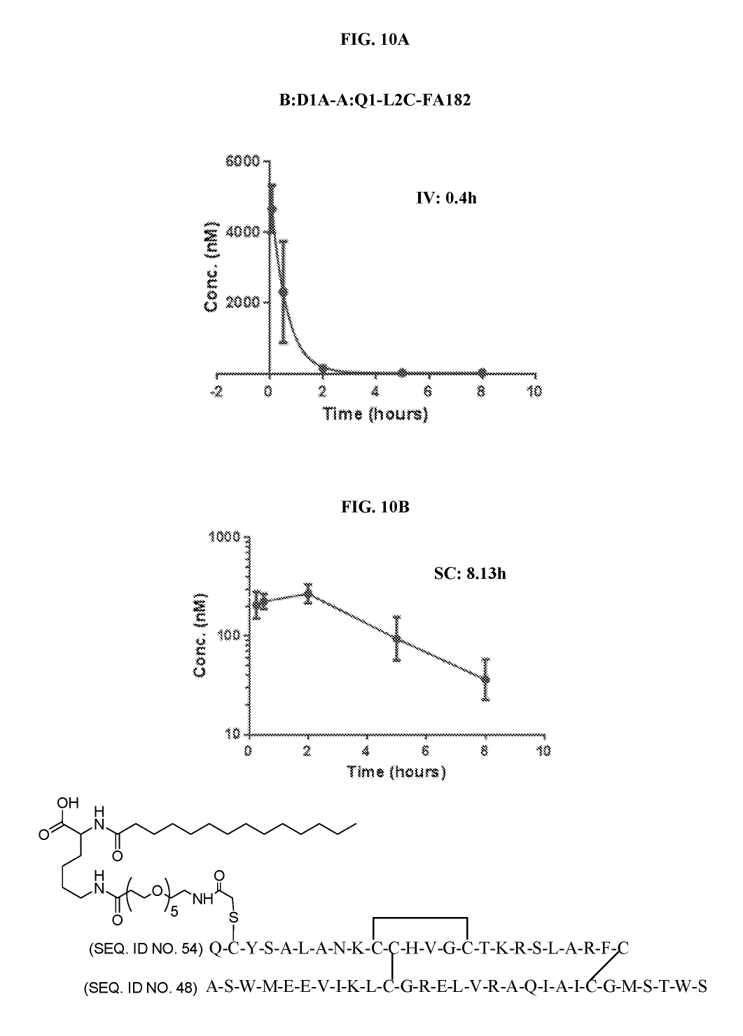

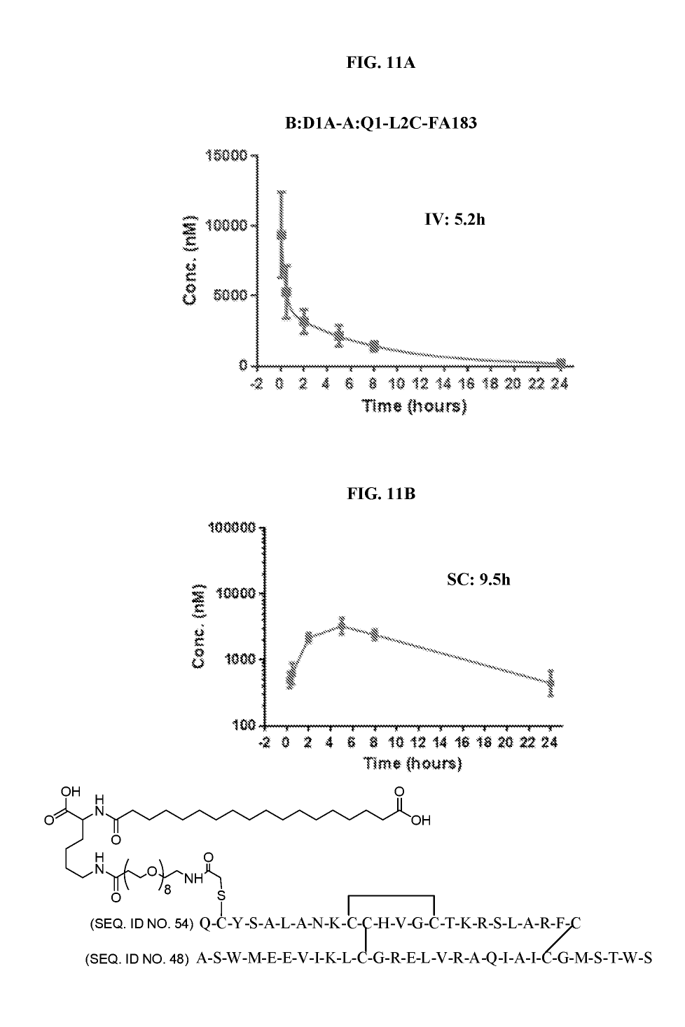

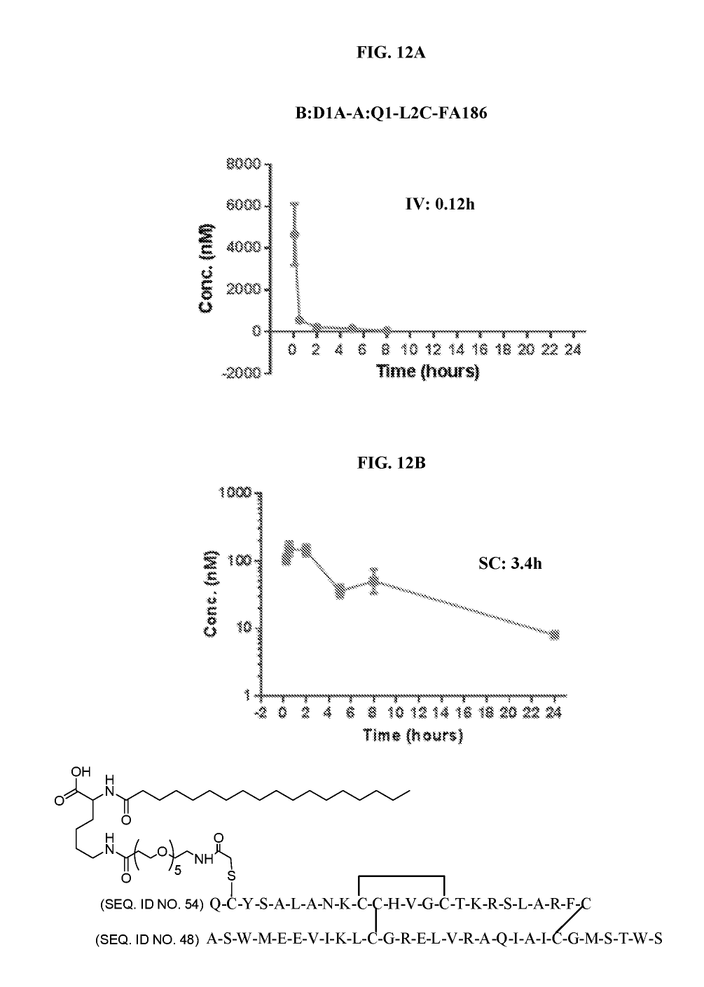

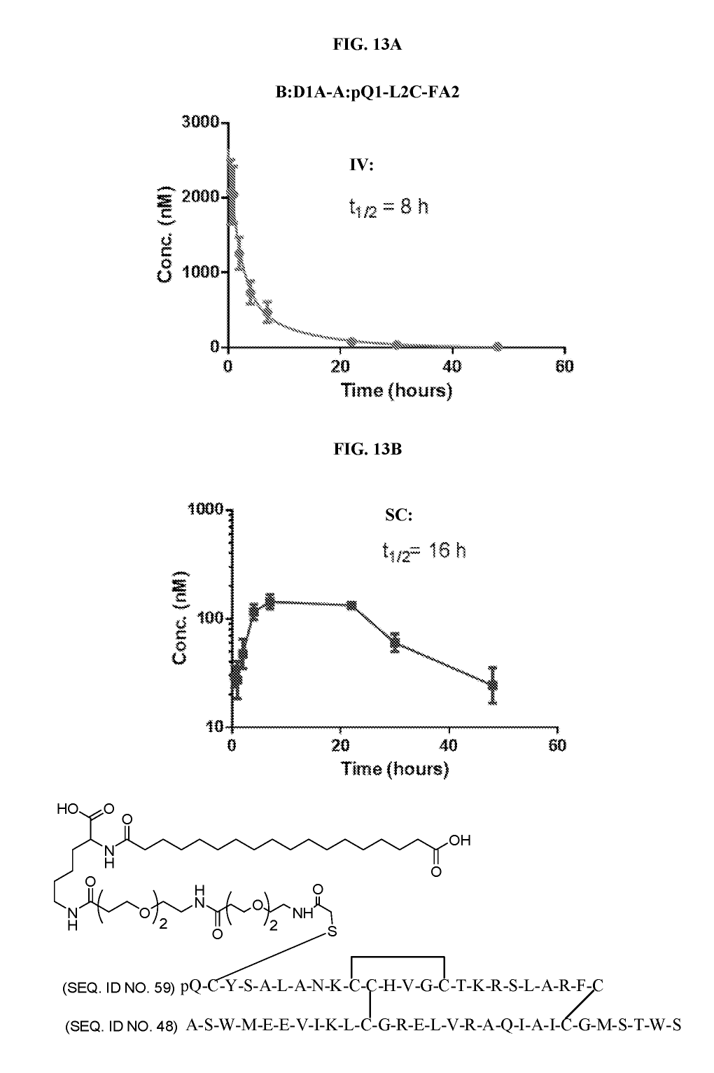

Lipid conjugated relaxin or other therapeutic agents were dosed in mice using sc, po, iv dosing. Plasma levels of TA were determined by LGR7 activation at different time points (e.g., i.v. PK 5 min, 0.5h, 1h, 2h 4h, 8h, 24h, 48h). Select PK data are shown in table 3 and FIGS. 6-13.

TABLE 3

| | | | | Activity, | | | |
|---|---|---|---|---|---|---|---|
| | | | | fold drop | LGR7 | | |
| | | Mass | Mass | compared | EC₅₀ | Half-life | |
| No. | Name | Expected | found | to relaxin | (pM) | (mouse, sc) | |
| 1 | Relaxin-B:D1A- | 6727.1 | 6727.3 | 12 | 210 | 8 | h |
| | A:Q1-L2C-FA2 | | | | | | |
| 2 | Relaxin-B:D1A- | 6741.2 | 6741.48 | 15 | 250 | 8 | h |
| | A:Q1:V13C-FA2 | | | | | | |
| 3 | Relaxin-B:D1A- | 6710.2 | 6710.41 | 12 | 210 | 6 | h |
| | A:Q1-L2C-FA155 | | | | | | |
| 4 | Relaxin-B:D1A- | 6241.4 | 6241.71 | 1 | 17~50 | NT | |
| | A:Q1-L2C-ALK | | | | | | |
| 5 | Relaxin-B:D1A- | 6710.1 | 6710.3 | 12 | 350 | 16 h (rat) | |
| | A:pQl-L2C-FA2 | | | | | | |
| 6 | Relaxin-B:D1A- | 8086.3 | 8086.5 | NT | NT | NT | |
| | A:Q1-L2C-FA175 | | | | | | |
| 7 | Relaxin-B:D1A- | 6614.1 | 6614.2 | 2 | 51 | 8 | h |
| | A:Q1-L2C-FA182 | | | | | | |
| 8 | Relaxin-B:D1A- | 6832.2 | 6832.4 | 8 | 174 | 9.5 | h |
| | A:Q1-L2C-FA183 | | | | | | |
| 9 | Relaxin-B:D1A- | 6669.9 | 6670.1 | 10 | 235 | 3.4 | h |
| | A:Q1-L2C-FA186 | | | | | | |
| 10 | Relaxin-B:D1A | 5936.1 | 5936.92 | 1 | 45 | 0.8 | h |
| 10 | Relaxin-wt | 5980.3 | 5980.9 | 1 | ~40 | 0.7 | h |

NT: not tested

### Example 36. Pharmacodynamic (PD) Study and In Vivo Efficacy in Acute Heart Failure Model

After determination of the PK, appropriate dose regimen are determined and lipid conjugated relaxin are dosed using sc, po or iv to evaluate the efficacy of lipid conjugated relaxin in efficacy models for blood pressure, urine flow and/or ligament elongation. Also, lipid conjugated relaxin is evaluated in bleomycin induced fibrosis model in lung and liver and/or in diabetic wound healing models in old diabetic zucker fat rats.

### Example 37. PD Study Using Mouse Interpubic Ligament Bioassay

The bioactivities of relaxin analogs are determined in CD1 mice by employing the well-established and highly specific mouse interpubic ligament bioassays for relaxin as described by Steinetz et al. [Endocrinology (1960) 67:102-115]. Female CD1 virgin mice at 3-4 weeks old are chosen from the study. Animals are group-housed under controlled temperature (25° C.) and photoperiod (12:12-hour light-dark cycle) conditions, and given unrestricted access to standard diet and tap water (or specified drinking solution). On day 1 of treatment, mice are s.c. injected with 10 μg estradiol cypionate in 0.1 ml sesame oil. On day 8, mice are divided into different groups: vehicle control, wt relaxin (dissolved in a suspension of 1% benzopurpurine 4B in PBS buffer) and relaxin analogs (dissolved in 20 mM NH₄Ac buffer, pH 6.5). Wt relaxin or relaxin analogs are administered at different dose through s.c. injection. At different time after relaxin injection, mice are euthanized and the length of interpubic ligaments is determined using caliper measurement.

### Example 38. Efficacy and Safety of Lipid Conjugated Relaxin for the Treatment of Acute Heart Failure

Purpose: Different doses of lipid conjugated relaxin are compared to placebo to determine efficacy and safety for the treatment of patients hospitalized with acute heart failure.

| Condition | Intervention | Phase |
|---|---|---|
| Heart Failure, Congestive | Drug: Relaxin | Phase 1 |
| | Drug: Placebo | Phase 1 |

Study Type: Interventional

Study Design: Allocation: Randomized

Endpoint Classification: Safety/Efficacy Study

Intervention Model: Parallel Assignment

Masking: Double Blind (Subject, Caregiver, Investigator, Outcomes Assessor)

Primary Purpose: Treatment

Primary Outcome Measures: Relief of dyspnea in acute heart failure [Time Frame: Up to day 5] [Designated as safety issue: No]

Secondary Outcome Measures:

Days alive and out of hospital [Time Frame: Up to day 60] [Designated as safety issue: No]

CV death or rehospitalization due to heart failure or renal failure [Time Frame: Up to day 60] [Designated as safety issue: No]

| Arms | Assigned Intervention |
|---|---|
| Placebo Comparator: Placebo | Drug: Placebo |
| 48 hour iv infusion of placebo | Intravenous infusion for 48 h |
| Experimental: Lipid Conjugated Relaxin | Drug: Lipid Conjugated Relaxin |
| 48 hour iv infusion of lipid conjugated | Intravenous infusion for 48 h |
| relaxin at 30 μg/kg/day | at 30 μg/kg/day |

### DETAILED DESCRIPTION

This is an international, randomized, double-blind, placebo-controlled, Phase II/III trial of intravenous recombinant relaxin for the treatment of signs and symptoms in patients hospitalized for acute decompensated heart failure. The Phase II pilot study has completed; the Phase III main portion of the trial is ongoing.

Eligibility

Ages Eligible for Study: 18 Years and older

Genders Eligible for Study: Both

Accepts Healthy Volunteers: No

Criteria

Inclusion Criteria:

Hospitalized for acute heart failure

Dyspnea at rest or with minimal exertion

Pulmonary congestion

Able to provide informed consent

Systolic blood pressure>125 mmHg

Impaired renal function defined as an eGFR of 30-75 mL/min/1.73m2

Exclusion Criteria:

Use of other IV therapies for acute heart failure

Fever or sepsis

Recent major neurologic event

Recent major surgery

Recent acute coronary syndrome

Other recent investigational drug use

TABLE 4
Therapeutic Agents (TAs) -Nucleotide Sequences

| | SEQ ID | |
|---|---|---|
| NAME | NO | SEQUENCE |
| Oxyntomodulin | 1 | CACTCTCAGGGTACCTTCACCTCTGACTACTCTAAATAC |
| | | CTGGACTCTCGTCGTGCTCAGGACTTCGTTCAGTGGCTG |
| | | ATGAACACCAAACGTAACCGTAACAACATCGCT |
| Relaxin | 2 | GACTCTTGGATGGAAGAAGTTATCAAACTGTGCGGTCGT |
| | | GAACTGGTTCGTGCTCAGATCGCTATCTGCGGTATGTCT |
| | | ACCTGGTCTGGTGGCGGTCGTGGCGGTCGTCAGCTGTAC |
| | | TCTGCTCTGGCTAACAAATGCTGCCACGTTGGTTGCACC |
| | | AAACGTTCTCTGGCTCGTTTCTGCTAA |
| Relaxin A Chain | 3 | CTGTACTCTGCTCTGGCTAACAAATGCTGCCACGTTGGT |
| | | TGCACCAAACGTTCTCTGGCTCGTTTCTGC |
| Relaxin SB29C | 4 | GACTCTTGGATGGAAGAAGTTATCAAACTGTGCGGTCGT |
| | | GAACTGGTTCGTGCTCAGATCGCTATCTGCGGTATGTCT |
| | | ACCTGGTGC |
| Leptin | 5 | GTTCCGATCCAGAAAGTTCAGGACGACACCAAAACCCT |
| | | GATCAAAACCATCGTTACCCGTATCAACGACATCTCTCA |
| | | CACCCAGTCTGTTTCTGCTAAACAGCGTGTTACtGGTCTG |
| | | GACTTCATCCCGGGTCTGCACCCGATCCTGTCTCTGTCTA |
| | | AAATGGACCAGACCCTGGCTGTTTACCAGCAGGTTCTGA |
| | | CCTCTCTGCCGTCTCAGAACGTTCTGCAGATCGCTAACG |
| | | ACCTGGAAAACCTGCGTGACCTGCTGCACCTGCTGGCTT |
| | | TCTCTAAATCTTGCTCTCTGCCGCAGACCTCTGGTCTGCA |
| | | GAAACCGGAATCTCTGGACGGTGTTCTGGAgGCTTCTCT |
| | | GTACTCTACCGAAGTTGTTGCTCTGTCTCGTCTGCAGGG |
| | | TTCTCTGCAGGACATCCTGCAGCAGCTGGACGTTTCTCC |
| | | GGAATGCTAA |
| Betatrophin | 6 | GCTCCTCTGGGCGGTCCTGAACCAGCACAGTACGAGGA |
| | | ACTGACACTGTTGTTCCATGGAGCCTTGCAGCTGGGCCA |
| | | GGCCCTCAACGGCGTGTACCGCGCCACAGAGGCACGTTT |
| | | GACCGAGGCCGGACACAGCCTGGGTTTGTACGACAGAG |
| | | CCCTGGAGTTTCTGGGTACCGAAGTGCGTCAGGGCCAGG |
| | | ACGCAACTCAGGAGCTGAGAACCTCCCTCTCTGAGATCC |
| | | AGGTGGAGGAGGACGCCCTGCACCTGCGCGCCGAGGCG |
| | | ACAGCACGCTCTTTGGGAGAAGTTGCTCGCGCTCAGCAG |
| | | GCCCTGCGTGATACCGTGCGGAGACTCCAAGTTCAGCTC |
| | | AGAGGCGCTTGGCTCGGACAGGCGCATCAGGAGTTCGA |
| | | GACCCTGAAAGCTCGTGCCGACAAACAGTCCCACCTGCT |
| | | GTGGGCGCTCACCGGTCACGTCCAGCGCCAGCAACGCG |
| | | AAATGGCCGAGCAGCAGCAATGGCTGCGCCAAATCCAG |
| | | CAGCGCCTGCATACCGCGGCCCTGCCAGCGTAA |
| FGF 21 | 7 | CACCCGATCCCGGACTCTTCTCCGCTGCTGCAGTTCGGT |
| | | GGTCAGGTTCGTCAGCGTTACCTGTACACCGACGACGCT |
| | | CAGCAGACCGAAGCTCACCTGGAAATCCGTGAAGACGG |
| | | TACtGTTGGTGGTGCTGCTGACCAGTCTCCGGAATCTCTG |
| | | CTGCAGCTGAAAGCTCTGAAACCGGGTGTTATCCAGATC |
| | | CTGGGTGTTAAAACCTCTCGTTTCCTGTGCCAGCGTCCG |
| | | GACGGTGCTCTGTACGGTTCTCTGCACTTCGACCCGGAG |
| | | GCATGCTCTTTCCGTGAACGTCTGCTGGAAGACGGTTAC |
| | | AACGTTTACCAGTCTGAAGCTCACGGTCTGCCGCTGCAC |
| | | CTGCCGGGTAACAAATCTCCGCACCGTGACCCGGCTCCG |
| | | CGTGGTCCGGCTCGTTTCCTGCCGCTGCCGGGTCTGCCG |
| | | CCGGCTCTGCCGGAACCGCCGGGTATCCTGGCTCCGCAG |
| | | CCGCCGGACGTTGGTTCTTCTGACCCGCTGTCTATGGTT |
| | | GGTGGTTCTCAGGGTCGTTCTCCGTCTTACGAATCTCCGT |
| | | AA |
| GDF 11 | 8 | AACCTGGGTCTGGACTGCGACGAACACTCTTCTGAATCT |
| | | CGTTGCTGCCGTTACCCGCTGACCGTTGACTTCGAGGCG |
| | | TTCGGTTGGGACTGGATCATCGCTCCGAAACGTTACAAA |
| | | GCTAACTACTGCTCTGGTCAGTGCGAATACATGTTCATG |
| | | CAGAAATACCCGCACACCCACCTGGTTCAGCAGGCTAA |
| | | CCCGCGTGGTTCTGCTGGTCCGTGCTGCACCCCGACCAA |
| | | AATGTCTCCGATCAACATGCTGTACTTCAACGACAAACA |
| | | GCAGATCATCTACGGTAAAATCCCGGGTATGGTTGTTGA |
| | | CCGTTGCGGTTGCTCTTAA |
| ANGPTL3 | 9 | GGATCCGGTGGTTTCACCATCAAACTGCTGCTGTTCATC |
| | | GTTCCGCTGGTTATCTCTTCTCGTATCGACCAGGACAAC |
| | | TCTTCTTTCGACTCTCTGTCTCCGGAACCGAAATCTCGTT |
| | | TCGCTATGCTGGACGACGTTAAAATCCTGGCTAACGGTC |
| | | TGCTGCAGCTGGGTCACGGTCTGAAAGACTTCGTTCACA |
| | | AAACCAAAGGTCAGATCAACGACATCTTCCAGAAACTG |
| | | AACATCTTCGACCAGTCTTTCTACGACCTGTCTCTGCAG |
| | | ACCTCTGAAATCAAAGAAGAAGAAAAAGAACTGCGTCG |
| | | TACCACCTACAAACTGCAGGTTAAAAACGAAGAAGTTA |
| | | AAAACATGTCTCTGGAACTGAACTCTAAACTGGAATCTC |
| | | TGCTGGAAGAAAAAATCCTGCTGCAGCAGAAAGTTAAA |
| | | TACCTGGAAGAACAGCTGACCAACCTGATCCAGAACCA |
| | | GCCGGAAACCCCGGAACACCCGGAAGTTACCTCTCTGA |
| | | AAACCTTCGTTGAAAAACAGGACAACTCTATCAAAGAC |
| | | CTGCTGCAGACCGTTGAAGACCAGTACAAACAGCTGAA |
| | | CCAGCAGCACTCTCAGATCAAAGAAATCGAAAACCAGC |
| | | TGCGTCGTACCTCTATCCAGGAACCGACCGAAATCTCTC |
| | | TGTCTTCTAAACCGCGTGCTCCGCGTACCACCCCGTTCCT |
| | | GCAGCTGAACGAAATCCGTAACGTTAAACACGACGGTA |
| | | TCCCGGCTGAATGCACCACCATCTACAACCGTGGTGAAC |
| | | ACACCTCTGGTATGTACGCTATCCGTCCGTCTAACTCTC |
| | | AGGTTTTCCACGTTTACTGCGACGTTATCTCTGGTTCTCC |
| | | GTGGACCCTGATCCAGCACCGTATCGACGGTTCTCAGAA |
| | | CTTCAACGAAACCTGGGAAAACTACAAATACGGTTTCG |
| | | GTCGTCTGGACGGTGAATTCTGGCTGGGTCTGGAAAAAA |
| | | TCTACTCTATCGTTAAACAGTCTAACTACGTTCTGCGTAT |
| | | CGAACTGGAAGACTGGAAAGACAACAAACACTACATCG |
| | | AATACTCTTTCTACCTGGGTAACCACGAAACCAACTACA |
| | | CCCTGCACCTGGTTGCTATCACCGGTAACGTTCCGAACG |
| | | CTATCCCGAAGAAGAAGAAGAAAAAAAAGAAGAAGAA |
| | | AT |

TABLE 5
Therapeutic Agents-Amino acid sequences

| NAME | SEQ ID NO | SEQUENCE |
|---|---|---|
| Oxyntomodulin | 10 | HSQGTFTSDYSKYLDSRRAQDFVQWLMNTKRNRNNIA |
| Exendin-4 or | 11 | HGEGTFTSDLSKQMEEEAVRLFIEWLKNGGPSSGAPPPS |
| Exenatide | | |
| hGLP-1 | 12 | HAEGTFTSDVSSYLEGQAAKEFIAWLVKGR |
| Glucagon | 13 | HSQGTFTSDYSKYLDSRRAQDFVQWLMNT |
| Relaxin | 14 | DSWMEEVIKLCGRELVRAQIAICGMSTWSGGGRGGRQLY |
| | | SALANKCCHVGCTKRSLARFC |
| Relaxin A | 15 | LYSALANKCCHVGCTKRSLARFC |
| Chain | | |
| Relaxin SB29C | 16 | DSWMEEVIKLCGRELVRAQIAICGMSTWC |
| Leptin | 17 | VPIQKVQDDTKTLIKTIVTRINDISHTQSVSAKQRVTGLDFI |
| | | PGLHPILSLSKMDQTLAVYQQVLTSLPSQNVLQIANDLEN |
| | | LRDLLHLLAFSKSCSLPQTSGLQKPESLDGVLEASLYSTEV |
| | | VALSRLQGSLQDILQQLDVSPEC |
| Betatrophin | 18 | APLGGPEPAQYEELTLLFHGALQLGQALNGVYRATEARL |
| | | TEAGHSLGLYDRALEFLGTEVRQGQDATQELRTSLSEIQV |
| | | EEDALHLRAEATARSLGEVARAQQALRDTVRRLQVQLRG |
| | | AWLGQAHQEFETLKARADKQSHLLWALTGHVQRQQRE |
| | | MAEQQQWLRQIQQRLHTAALPA |
| FGF 21 | 19 | HPIPDSSPLLQFGGQVRQRYLYTDDAQQTEAHLEIREDGT |
| | | VGGAADQSPESLLQLKALKPGVIQILGVKTSRFLCQRPDG |
| | | ALYGSLHFDPEACSFRERLLEDGYNVYQSEAHGLPLHLPG |
| | | NKSPHRDPAPRGPARFLPLPGLPPALPEPPGILAPQPPDVGS |
| | | SDPLSMVGGSQGRSPSYESP |
| GDF 11 | 20 | NLGLDCDEHSSESRCCRYPLTVDFEAFGWDWIIAPKRYKA |
| | | NYCSGQCEYMFMQKYPHTHLVQQANPRGSAGPCCTPTK |
| | | MSPINMLYFNDKQQIIYGKIPGMVVDRCGCS |
| ANGPTL3 | 21 | GSGGFTIKLLLFIVPLVISSRIDQDNSSFDSLSPEPKSRFAML |
| | | DDVKILANGLLQLGHGLKDFVHKTKGQINDIFQKLNIFDQ |
| | | SFYDLSLQTSEIKEEEKELRRTTYKLQVKNEEVKNMSLEL |
| | | NSKLESLLEEKILLQQKVKYLEEQLTNLIQNQPETPEHPEV |
| | | TSLKTFVEKQDNSIKDLLQTVEDQYKQLNQQHSQIKEIEN |
| | | QLRRTSIQEPTEISLSSKPRAPRTTPFLQLNEIRNVKHDGIPA |
| | | ECTTIYNRGEHTSGMYAIRPSNSQVFHVYCDVISGSPWTLI |
| | | QHRIDGSQNFNETWENYKYGFGRLDGEFWLGLEKIYSIVK |
| | | QSNYVLRIELEDWKDNKHYIEYSFYLGNHETNYTLHLVAI |
| | | TGNVPNAIPKKKKKKKKKK |
| Moka | 22 | INVKCSLPQQCIKPCKDAGMRFGKCMNKKCRCYS |
| VM-24 | 23 | AAAISCVGSPECPPKCRAQGCKNGKCMNRKCKCYYC |
| Oxm-Cys38 | 24 | HsQGTFTSDYSKYLDSRRAQDFVQWLMNTKRNRNNIAC |
| Ex4-Cys40 | 25 | HGEGTFTSDLSKQMEEEAVRLFIEWLKNGGPSSGAPPPSC |
| Toxin H8-Tev- | 26 | HHHHHHHHENLYFQGSCGGECIGMFKSCDPENDKCCKGR |
| 550 | | TCSRKHRWCKYKL |
| 550-GSGG | 27 | GSGGEECIGMFKSCDPENDKCCKGRTCSRKHRWCKYKL |
| 550-4-GSCGG | 28 | GSCGGECIGMFKSCDPENDKCCKGRTCSRKHRWCKYKL |
| 550-3-GSGG | 29 | GSGGECIGMFKSCDPENDKCCKGRTCSRKHRWCKYKLC |
| Toxin-550 | 30 | ECIGMFKSCDPENDKCCKGRTCSRKHRWCKYKL |
| 550-3 | 31 | ECIGMFKSCDPENDKCCKGRTCSRKHRWCKYKLGGSC |
| Tev-Relaxin- | 32 | *HHHHHHHHENLYFQGSGGDSWMEEVIKLCGRELVRAQI |
| single-chain | | AICGMSTWSGGGRGGRQLYSALANKCCHVGCTKRSLARF |
| | | C* |
| GSGG-Relaxin- | 33 | *GSGGDSWMEEVIKLCGRELVRAQIAICGMSTWSGGG |
| single chain | | RGGRQLYSALANKCCHVGCTKRSLARFC* |
| Tev-Relaxin-C- | 34 | *HHHHHHHHENLYFQGSGGDSWMEEVIKLCGRELVRA |
| chain | | QIAICGMSTWSKRSLSQEDAPQTPRPVAEIVPSFINKDTETI |
| | | NMMSEFVANLPQELKLTLSEMQPALPQLQQHVPVLKDSS |
| | | LLFEEFKKLIRNRQSEAADSSPSELKYLGLDTHSRKKRQLY |
| | | SALANKCCHVGCTKRSLARFC* |
| Tev-Relaxin | 35 | *HHHHHHHHENLYFQSGGDSWMEEVIKLCGRELVRAQI |
| Chain 1 | | AICGMSTWS* |
| Tev-Relaxin | 36 | *QLYSALANKCCHVGCTKRSLARFC* |
| Chain 2 | | |
| Tev-Relaxin-B- | 37 | *HHHHHHHHENLYFQGSGGDSWMEEVIKLCGRELVRAQI |
| S29C-single- | | AICGMSTWCGGGRGGRQLYSALANKCCHVGCTKRSLAR |
| chain | | FC* |
| Tev-Relaxin-B- | 38 | *HHHHHHHHENLYFQGSGGDSWMEEVIKLCGRELVRA |
| S29C-C-chain | | QIAICGMSTWCKRSLSQEDAPQTPRPVAEIVPSFINKDTETI |
| | | NMMSEFVANLPQELKLTLSEMQPALPQLQQHVPVLKDSS |
| | | LLFEEFKKLIRNRQSEAADSSPSELKYLGLDTHSRKKRQLY |
| | | SALANKCCHVGCTKRSLARFC* |
| Pro-relaxin-B- | 39 | *MIEEGRDSWMEEVIKLCGRELVRAQIAICGMSTWSKRKP |
| D1A | | TGYGSRKKRQLYSALANKCCHVGCTKRSLARFC* |
| B-chain | 40 | DSWMEEVIKLCGRELVRAQIAICGMSTWS |
| A-chain | 41 | LYSALANKCCHVGCTKRSLARFC |
| Relaxin A chain | 42 | QLYSALANKCCHVGCTKRSLARFC |
| 1 | | |
| Relaxin A chain | 43 | QLYSCLANKCCHVGCTKRSLARFC |
| 2 | | |
| Relaxin A chain | 44 | QLYSALANKCCKVGCTKRSLARFC |
| 3 | | |
| Relaxin A chain | 45 | CLYSALANKCCHVGCTKRSLARFC |
| 4 | | |
| Relaxin A chain | 46 | ALYSALANKCCAVGCTKRSLARFC |
| 5 | | |
| Relaxin A chain | 47 | ALYSALANKCCAVGCTKRSLARFC |
| 6 | | |
| Relaxin B chain | 48 | ASWMEEVIKLCGRELVRAQIAICGMSTWS |
| 1 | | |
| Relaxin B chain | 49 | ASWMEEVIKLCGRELVRAQIAICGMSTWC |
| 2 | | |
| Relaxin B chain | 50 | ASWKEEVIKLCGRELVRAQIAICGKSTWC |
| 3 | | |
| Relaxin B chain | 51 | ASWKEEVIKLCGRELVRAQIAICGMSTWC |
| 4 | | |
| Relaxin B chain | 52 | CSWMEEVIKLCGRELVRAQIAICGMSTWS |
| 5 | | |
| H2-Relaxin A | 53 | ZLYSALANKCCHVGCTKRSLARFC |
| chain 1 | | |
| H2-Relaxin A | 54 | QCYSALANKCCHVGCTKRSLARFC |
| chain 2 | | |
| H2-Relaxin A | 55 | QLYSALANKCCHCGCTKRSLARFC |
| chain 3 | | |
| H2-Relaxin B | 56 | DSWMEEVIKCLGRELVRAQIAICGMSTWS |
| chain | | |
| Relaxin A chain | 57 | QLYSALANKCCHVGCTKCSLARFC |
| 7 | | |
| Relaxin B chain | 58 | ASWMEEVIKLCGRELVRAQIAICGKSTWC |
| 6 | | |
| Relaxin A chain | 59 | pQCYSALANKCCHVGCTKRSLARFC |
| 8 | | |
| Relaxin A chain | 60 | X¹X²YSX³LANKCCX⁴X⁵GCTKX⁶SLARFC, |
| 9 | | wherein: X¹ = Z or A; X² = C, L, A, I, or V; |
| | | X³ = C, L, A, I, or V; X⁴ = H, A or K; |
| | | X⁵ = C, L, A, I, or V; X⁶ = R or C; wherein at |
| | | least one of X², X³, X⁵ or X⁶ is C. |
| Relaxin B chain | 61 | ASWMEECIKLCGRELVRAQIAICGMSTWS |
| 7 | | |

Lowercase letters represent D-amino acids
Tev = Tev protease cleavage sequence (ENLYFQ (SEQ ID NO: 71))
* = free N-terminal or unmodified C-terminal carboxyl group
Z = Q, E, or pyroglutamate (pQ)

TABLE 6
Relaxin2 Construction Primer Sequences

| NAME | SEQ ID NO | SEQUENCE |
|---|---|---|
| Relaxin2 f1 | 62 | TATTTCCAGGGATCCGGTGGTGACTCTTGGATGGAAGA |
| (#32) | | AGTTATCAAACTGTGCGGTCGT |
| Relaxin2 r6 | 63 | TAGACATACCGCAGATAGCGATCTGAGCACGAACCAG |
| linker S26C | | TTCACGACCGCACAGTTTGATAA |
| (#212) | | |
| Relaxin2 Linker | 64 | CGCTATCTGCGGTATGTCTACCTGGTCTGGTGGCGGTC |
| f1 S26C(#213) | | GTGGCGGTCGTCAGCTGTACTC |
| Relaxin2 Linker | 65 | CGCTATCTGCGGTATGTCTACCTGGTCTGGTGGCGGTC |
| f1 (#209) | | GTGGCGGTCGTCAGCTGTACTC |
| Relaxin2 r6 | 66 | TAGACATACCGCAGATAGCGATCTGAGCACGAACCAG |
| (#43) | | TTCACGACCGCACAGTTTGATAA |
| Relaxin2 Linker | 67 | CGCTATCTGCGGTATGTCTACCTGGTCTGGTGGCGGTC |
| f1 S29C(#214) | | GTGGCGGTCGTCAGCTGTACTC |
| Relaxin2 Linker | 68 | TTTGGTGCAACCAACGTGGCAGCATTTGTTAGCCAGAG |
| r1 (#210) | | CAGAGTACAGCTGACGACCGCC |
| pVB008 | 69 | AATCTGTATTTCCAGGGATCCGGTGGTGA |
| Relaxin2 Amp | | |
| for (#60) | | |
| Relaxin2 Linker | 70 | TGGCTAAGCTTTAGCAGAAACGAGCCAGAGAACGTTT |
| Amp rev (#211) | | GGTGCAACCAACGTGGC |

1.-11. (canceled)

12. A modified therapeutic agent comprising:
(i) a therapeutic agent (TA) comprising a relaxin A-chain amino acid sequence that is at least 90% homologous to an amino acid sequence selected from the group consisting of SEQ ID NO: 54, 55, 59 and 60; and
(ii) a half-life extending moiety;
wherein the TA is covalently attached to the half-life extending moiety via a cysteine residue of the TA.

13. (canceled)

14. The modified therapeutic agent of claim 12, wherein the relaxin A-chain amino acid sequence is selected from the group consisting of SEQ ID NO: 54, 55, 59 and 60.

15. The modified therapeutic agent of claim 12, wherein the cysteine residue is in the relaxin A-chain amino acid sequence.

16. (canceled)

17. (canceled)

18. (canceled)

19. The modified therapeutic agent of claim 12, wherein the therapeutic agent further comprises a relaxin B-chain.

20. (canceled)

21. (canceled)

22. The modified therapeutic agent of claim 12, wherein the therapeutic agent further comprises a relaxin B-chain amino acid sequence that is at least 90% homologous to an amino acid sequence selected from the group consisting of SEQ ID NO: 16, 40, 48-52, 56, 58, and 61.

23. The modified therapeutic agent of claim 12, wherein the therapeutic agent further comprises a relaxin B-chain amino acid sequence SEQ ID NO: 48.

24. The modified therapeutic agent of claim 12, wherein the half-life of the modified therapeutic agent is longer than the half-life of the therapeutic agent alone.

25.-52. (canceled)

53. The modified therapeutic agent of claim 12, wherein the modified therapeutic agent has the following formula:
TA-A¹-P¹-L Formula (A)
wherein:
TA is the therapeutic agent; 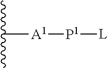
is the half-life extending moiety.

54. The modified therapeutic agent of claim 53, wherein:
A¹ is a chemical group linking TA and P¹ or L;
P¹ is a bond or -PEG-A²-; and
L is a lipid derivative selected from the group consisting of sterol derivatives, bile acid derivatives, vitamin E derivatives, fatty di-acid derivatives, fatty acid derivatives, fatty amide derivatives, fatty amine amine derivatives, and fatty alcohol derivatives; wherein the lipid derivative is linked to P¹ via an amide or an ether linkage.

55. (canceled)

56. The modified therapeutic agent of claim 54, wherein
A¹ is selected from 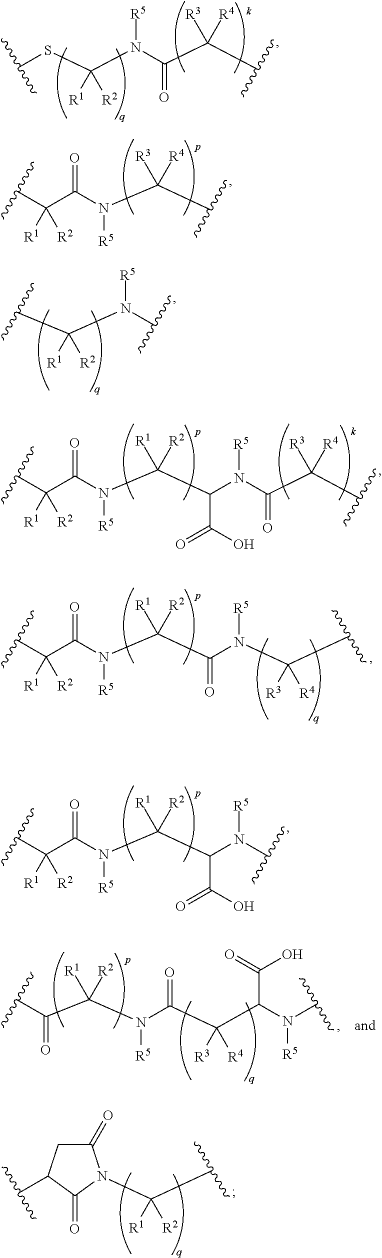
PEG is selected from 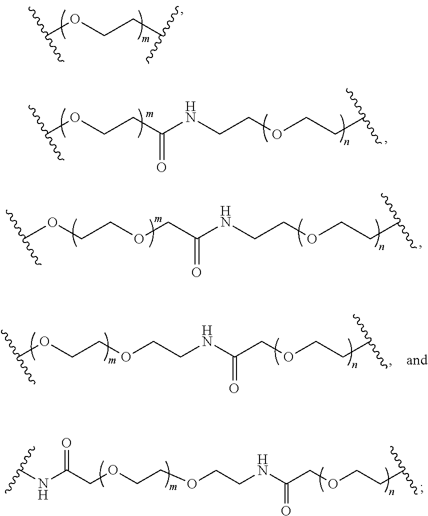
A² is selected from a bond, 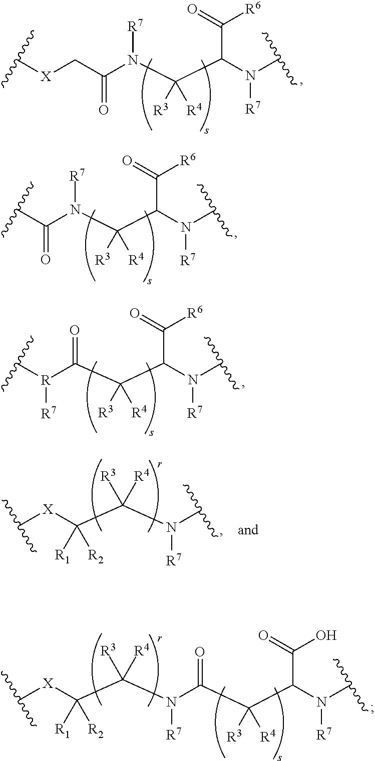
L is an acid moiety selected from tetradecanoic acid, octadecanedioic acid, myristic acid, stearic acid, docosahexaenoic acid, lithocholic acid, cholic acid, and palmitic acid; wherein the acid moiety is linked to P¹ via an amide linkage;
X is a bond, —N(R⁵)—, —S—, or —O—;
each R¹, R², R³, and R⁴ is independently H, halo, —CN, —SR⁵, alkyl, cycloalkyl, haloalkyl, —NR⁵R⁵, or —OR⁵;
each R⁵ is independently H, alkyl, haloalkyl, arylalkyl, or heteroalkyl;
R⁶ is OH or —NR⁵R⁵;
each R⁷ is independently H, alkyl, haloalkyl, arylalkyl, or heteroalkyl;
m and n are independently 1, 2, 3, 4, 5, 6, 7, 8, 9, 10, 11, 12, 13, 14, 15, 16, 17, 18, 19, or 20;
r is 1, 2, 3, 4, 5, 6, 7, 8, 9, or 10;
s is 1, 2, 3, 4, or 5;
k is 1, 2, 3, 4, 5, 6, 7, 8, 9, or 10;
p is 2, 3, 4, 5, 6, 7, 8, 9, or 10; and
q is 2, 3, 4, 5, 6, 7, 8, 9, or 10.

57. The modified therapeutic agent of claim 56, wherein:
A¹ is

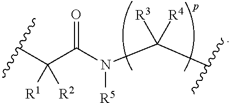

58. The modified therapeutic agent of claim 56, wherein:
PEG is

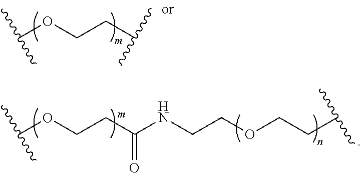

59. The modified therapeutic agent of claim 56, wherein:
A² is

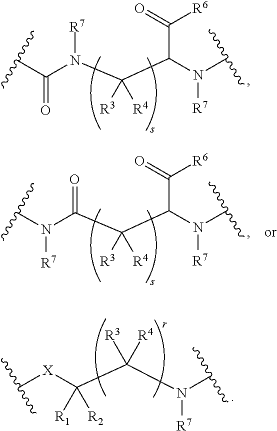

60. The modified therapeutic agent of claim 56, wherein
each R¹, R², R³, and R⁴ is H;
each R⁵ is independently H;
each R⁶ is OH or NH₂;
each R⁷ is H;
m is 1, 2, 3, 4, 5, 6, 7, 8, or 9;
n is 1, 2, 3, 4, or 5;
r is 1, 2, or 3;
s is 2 or 4;
p is 2; and
X is —O—.

61.-69. (canceled)

70. The modified therapeutic agent of claim 53, wherein the modified therapeutic agent is selected from the following:

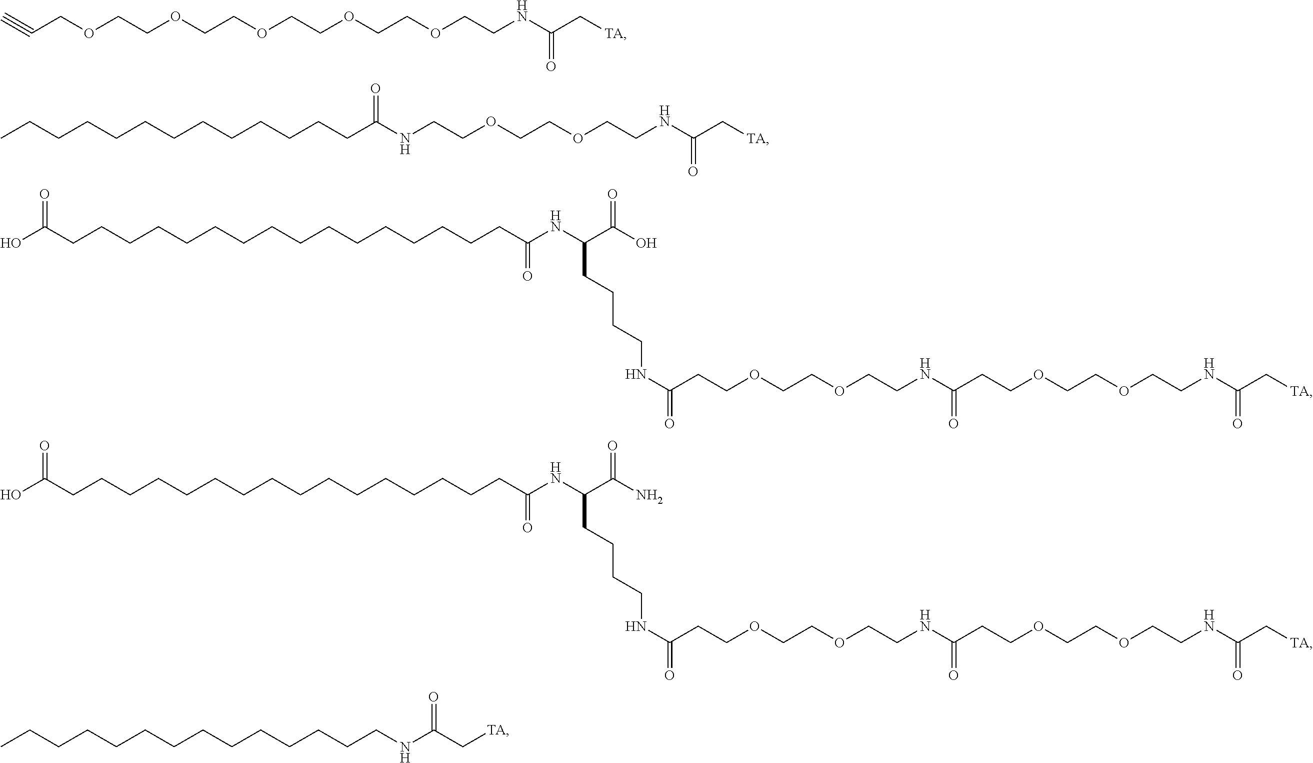

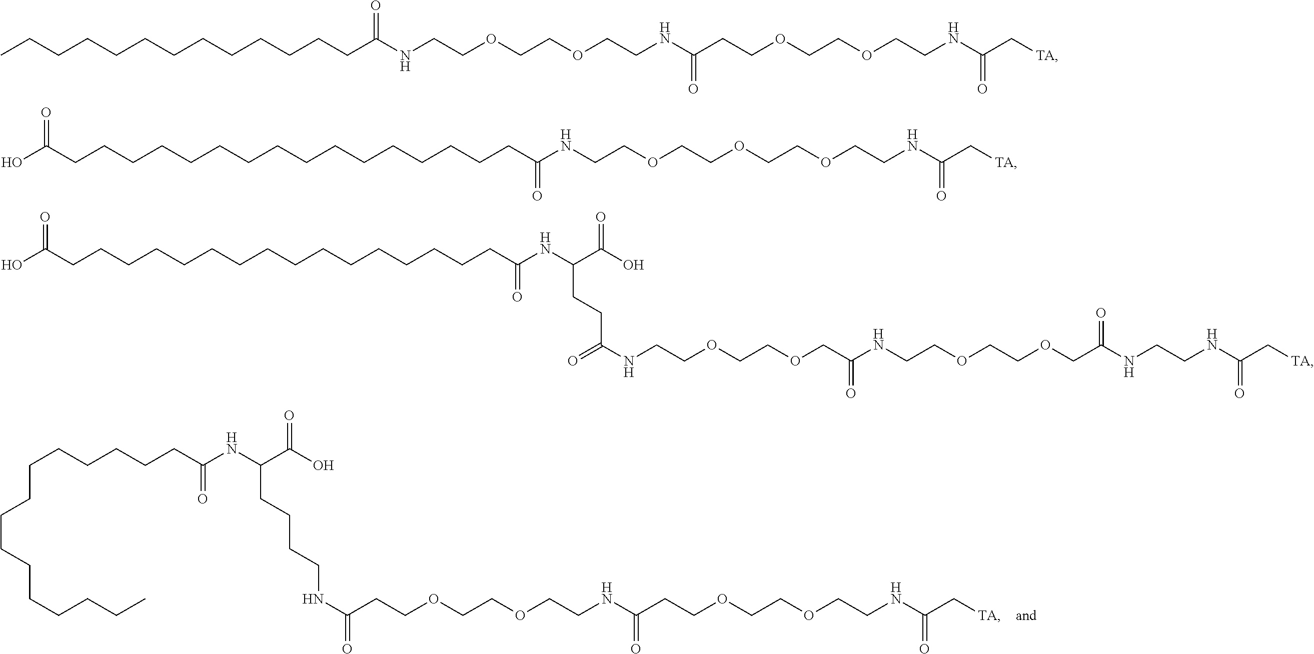

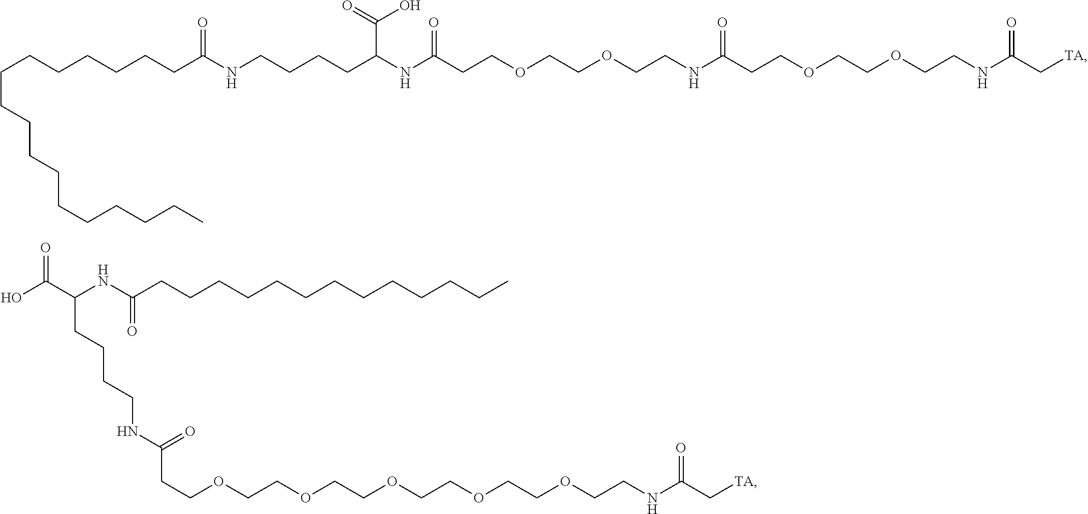

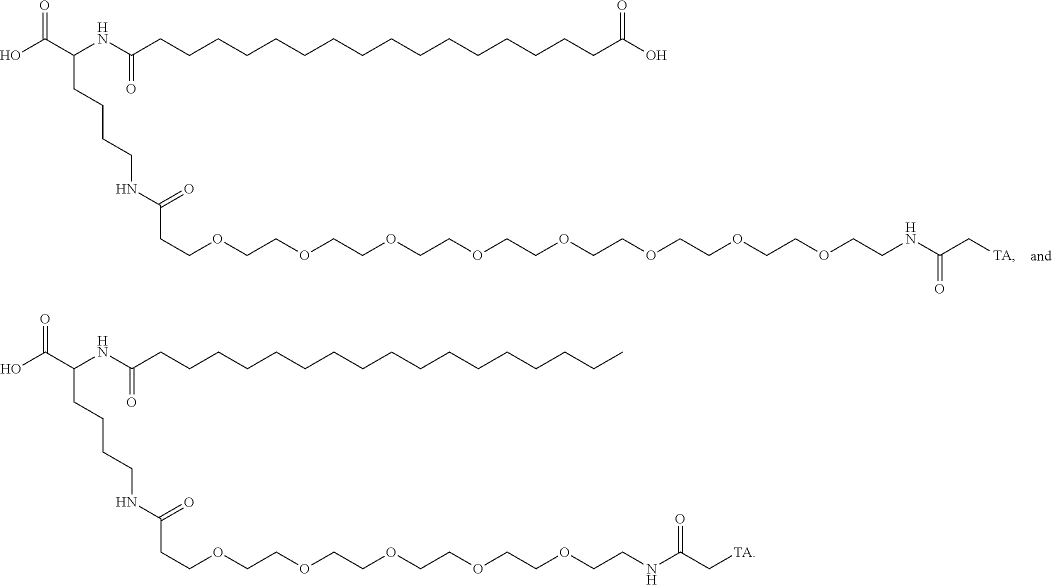

71. The modified therapeutic agent of claim 53, wherein the modified therapeutic agent is:

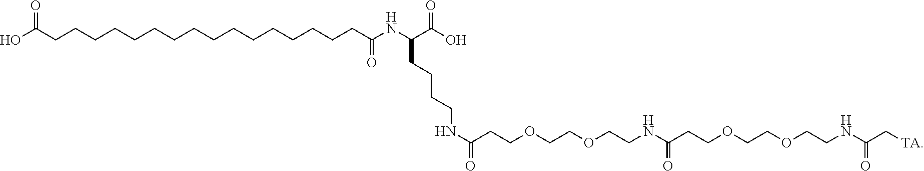

72. (canceled)

73. A pharmaceutical composition comprising the modified therapeutic agent of claim 12.

74. A method for treating a disease or condition in a subject in need thereof, the method comprising administering to the subject a composition comprising a therapeutically effective amount of the modified therapeutic agent of claim 12.

75. The method of claim 74, wherein the disease or condition is a cardiovascular disorder, acute heart failure, fibrosis, or pain.

76.-79. (canceled)

80. The method of claim 74, wherein the modified therapeutic agent is administered with one or more additional therapeutic agents selected from group consisting of an anti-inflammatory drug and a drug to treat pain, or any combination thereof.

81. (canceled)

82. (canceled)
